(19) United States
(12) Patent Application Publication
(10) Pub. No.: US 2025/0049514 A1
(43) Pub. Date: Feb. 13, 2025

(54) AUGMENTED REALITY GLASSES FOR ALIGNMENT OF APPARATUS IN SURGICAL PROCEDURE

(71) Applicant: Circinus Medical Technology LLC, Concord, MA (US)

(72) Inventor: John Kyle Dorman, Midland, TX (US)

(73) Assignee: Circinus Medical Technology LLC, Concord, MA (US)

(21) Appl. No.: 18/799,937

(22) Filed: Aug. 9, 2024

Related U.S. Application Data
(60) Provisional application No. 63/518,804, filed on Aug. 10, 2023.

(51) Int. Cl. A61B 34/20; A61B 34/10

(52) U.S. Cl. CPC A61B 34/20; A61B 2034/107; A61B 2034/2046

(57) ABSTRACT

Systems and methods are provided for orienting a surgical tool (or medical device) at a desired insertion angle and location within an environment using a smart headset for use in installing the medical device. In certain implementations, a method may include initiating a smart headset to be calibrated to the environment so that the position of the smart headset is known relative to the environment when the smart headset moves in the environment; receiving, by the smart headset from an electronic device, environmental data indicating the position of the surgical tool within the environment; receiving, by the smart headset from the electronic device, the desired insertion angle; generating, by the smart headset or otherwise, at least one graphical element for orienting the surgical tool at the desired insertion angle (such as a three-dimensional insertion angle) and location; and displaying the at least one graphical element superimposed within the environment.

### CROSS-REFERENCE TO RELATED PATENT APPLICATIONS

This applications claims priority to U.S. Provisional Application 63/518,804, filed Aug. 10, 2023, incorporated herein by reference in its entirety for any and all purposes.

### BACKGROUND

When images are captured using an image capture device, such as a camera, the angle in which the image is captured may skew or alter details of the image. This could, for example, cause unintended consequences if such altered details are used in connection with images used for medical procedures or for diagnoses. For example, in connection with spinal fusion surgery, these patients may an interbody cage between their vertebrae. The interbody cage can be implanted between the vertebrae from the back, front, or side of the patient. A pilot hole may be created through the body to create the path or tract through which an instrument will be inserted. Placing the instrument at the correct angle helps to ensure a mechanically sound construct and to avoid injury to surrounding structures such as the spinal cord, nerve roots, and blood vessels. The orientation of the interbody cage and its accompanying instrument (e.g., an inserter) can be described by a three-dimensional alignment angle or insertion angle, and the correct image capture of any diagnostic images used in determining such an alignment insertion angle needs to be properly and accurately performed.

Other situations in which having a true alignment and image capture of an object or the subject can be important. Examples include construction, interior design, CAD drawings, and three-dimensional printing. Another example, as mentioned above, is a surgical navigation system in which having a true and accurate angle is a prerequisite for safe functioning.

### SUMMARY

Some implementations relate to a method for orienting a surgical tool at a desired three-dimensional insertion angle at a desired location within an environment for use in installing a medical device by using and displaying at least one graphical element. The processing circuits can be configured to initiate a smart headset to be calibrated to the environment so that a position of the smart headset is known relative to the environment when the smart headset moves in the environment. The processing circuits can be configured to receive, by the smart headset from an electronic device communicatively coupled to the smart headset, environmental data indicating the position of the surgical tool within the environment. The processing circuits can be configured to receive, by the smart headset from the electronic device, the desired three-dimensional insertion angle. The processing circuits can be configured to generate, by the smart headset, at least one graphical element including visual indicia for orienting the surgical tool at the desired three-dimensional insertion angle at the desired location. The processing circuits can be configured to display, by the smart headset, the at least one graphical element superimposed within the environment.

In some implementations, the visual indicia can include a virtual tool for orienting the surgical tool at the desired location and the desired three-dimensional insertion angle. The visual indicia can further include a three-dimensional vector including a guideline indicating a trajectory of the virtual tool. The processing circuits can be configured to generate, by the smart headset, interactive elements for interacting with the smart headset and displaying, by the smart headset, the interactive elements superimposed within the environment.

In some implementations, the processing circuits can be configured to receive, by an input device of the smart headset, an instruction from an individual operating the smart headset. The processing circuits can be configured to lock, by the smart headset, the virtual tool superimposed within the environment. The virtual tool can be stationary at the desired location and the desired three-dimensional insertion angle as the smart headset changes positions within the environment. The instruction from the individual can be at least one of an eye movement, a gesture, an auditory pattern, a movement pattern, haptic feedback, a biometric input, intangible feedback, or a preconfigured interaction.

In some implementations, the visual indicia can include concentric circles indicating thresholds of the desired three-dimensional insertion angle of the surgical tool. The concentric circles can include a first set of concentric circles indicating the orientation of the surgical tool at the desired location based on the desired three-dimensional insertion angle and a second set of concentric circles indicating a live orientation of the surgical tool. The electronic device can be calibrated to the surgical tool to indicate the live orientation of the surgical tool. The environmental data can include orientation data of the surgical tool. The smart headset can continually receive the environmental data from the electronic device in real-time.

In some implementations, in response to continually receiving the environmental data, the processing circuits can be configured to automatically update, by the smart headset in real-time, the at least one graphical element superimposed within the environment. The smart headset can include a gyroscope, and generating and displaying the at least one graphical element can be based on continually collecting, by the gyroscope in real-time, orientation data of the smart headset. In response to continually collecting the orientation data of the smart headset, the processing circuits can be configured to automatically update, by the smart headset in real-time, the at least one graphical element superimposed within the environment.

In some implementations, the processing circuits can be configured to capture, by an input device of the smart headset, additional environmental data of the environment. The input device can be at least one of a camera, sensor, or internet of things (IoT) device. The additional environmental data can include orientation data of a portion of a body. The orientation data of the portion of the body can indicate at least one of an axial plane, coronal plane, or a sagittal plane associated with anatomy of the portion of the body. The processing circuits can be configured to determine an orientation of the portion of the body within the environment based on inputting the orientation data into a machine learning algorithm and receiving an output prediction indicating the orientation of the portion of the body within the environment.

In some implementations, generating the at least one graphical element including the visual indicia for orienting the surgical tool at the desired location can be further based on the orientation of the portion of the body within the environment. The processing circuits can be configured to generate visual indicator elements indicating the orientation of the portion of the body within the environment and displaying, by the smart headset, the visual indicator elements superimposed within the environment.

In some implementations, the surgical tool can be one of a gear shift probe, a pedicle probe, a Jamshidi needle, an awl, a tap, a screw inserter, a drill, or a syringe. The environmental data can include planning data for performing an operation at the desired location using the surgical tool. The processing circuits can be configured to receive and store diagnostic images of a portion of a body. Generating the at least one graphical element can be further based on the diagnostic images of the portion of the body.

Some implementations relate to a method for orienting a surgical tool at a desired three-dimensional insertion angle at a desired location within an environment for use in installing a medical device by using and displaying at least one graphical element. The processing circuits can be configured to determine the desired three-dimensional insertion angle of the surgical tool based on an orientation of the surgical tool. The processing circuits can be configured to collect environmental data of the surgical tool within the environment. The processing circuits can be configured to generate at least one graphical element including visual indicia for orienting the surgical tool at the desired location based on the desired three-dimensional insertion angle. The processing circuits can be configured to display, on a smart headset communicatively coupled to the processing circuits, the at least one graphical element superimposed within the environment.

In some implementations, the visual indicia can include a virtual tool for orienting the surgical tool at the desired location and the desired three-dimensional insertion angle. The visual indicia can further include a three-dimensional vector including a guideline indicating a trajectory of the virtual tool. The processing circuits can be configured to generate interactive elements for interacting with the smart headset and displaying, by the smart headset, the interactive elements superimposed within the environment. In some implementations, the one or more processors are enclosed within the smart headset

In some implementations, the processing circuits can be configured to receive, by an input device of the smart headset, an instruction from an individual operating the smart headset. The processing circuits can be configured to lock, by the smart headset, the virtual tool superimposed within the environment. The virtual tool can be stationary at the desired location and the desired three-dimensional insertion angle as the smart headset changes positions within the environment. The instruction from the individual can be at least one of an eye movement, a gesture, an auditory pattern, a movement pattern, haptic feedback, a biometric input, intangible feedback, or a preconfigured interaction.

In some implementations, the visual indicia can include concentric circles indicating thresholds of the desired three-dimensional insertion angle of the surgical tool. The concentric circles can include a first set of concentric circles indicating the orientation of the surgical tool at the desired location based on the desired three-dimensional insertion angle and a second set of concentric circles indicating a live orientation of the surgical tool. The processing circuits can be calibrated to the surgical tool to indicate the live orientation of the surgical tool. The environmental data can include orientation data of the surgical tool. The processing circuits can continually collect the environmental data in real-time.

In some implementations, in response to continually collecting the environmental data, the processing circuits can be configured to automatically update, by the smart headset in real-time, the at least one graphical element superimposed within the environment. The processing circuits can include a gyroscope, and generating and displaying the at least one graphical element can be based on continually collecting, by the gyroscope in real-time, orientation data of the smart headset. In response to continually collecting the orientation data of the smart headset, the processing circuits can be configured to automatically update, by the smart headset in real-time, the at least one graphical element superimposed within the environment.

In some implementations, the processing circuits can be configured to capture, by an input device of the smart headset, additional environmental data of the environment. The input device can be at least one of a camera, sensor, or internet of things (IoT) device. The additional environmental data can include orientation data of a portion of a body. The orientation data of the portion of the body can indicate at least one of an axial plane, coronal plane, or a sagittal plane associated with anatomy of the portion of the body. The processing circuits can be configured to determine an orientation of the portion of the body within the environment based on inputting the orientation data into a machine learning algorithm and receiving an output prediction indicating the orientation of the portion of the body within the environment.

In some implementations, generating the at least one graphical element including the visual indicia for orienting the surgical tool at the desired location can be further based on the orientation of the portion of the body within the environment. The processing circuits can be configured to generate visual indicator elements indicating the orientation of the portion of the body within the environment and displaying, by the smart headset, the visual indicator elements superimposed within the environment. The processing circuits can be configured to receive and store diagnostic images of a portion of the body. Generating the at least one graphical element can be further based on the diagnostic images of the portion of the body.

Some implementations relate to a smart headset for orienting a tool at a desired location within an environment. The smart headset can include a transparent or opaque display, a plurality of sensor devices, and one or more processors configured to initiate the smart headset to be calibrated to the environment so that the smart headset knows its position relative to the environment when the smart headset moves in the environment. The one or more processors can be configured to collect, via the plurality of sensor devices, environmental data of a surgical tool within the environment using physical elements or fiducial markers or geometric shapes of the surgical tool that can be located at the desired location. The one or more processors can be configured to calculate an orientation of the surgical tool based on collecting the physical elements or fiducial markers or geometric shapes of the surgical tool. The one or more processors can be configured to receive a desired three-dimensional insertion angle. The one or more processors can be configured to determine the position of the desired three-dimensional insertion angle at the desired location. The one or more processors can be configured to generate at least one graphical element including visual indicia for orienting the surgical tool at the desired three-dimensional insertion angle at the desired location. The one or more processors can be configured to display, via the transparent or opaque display, the at least one graphical element superimposed within the environment.

In some implementations, the visual indicia can include a virtual tool for orienting the surgical tool at the desired location and the desired three-dimensional insertion angle. The visual indicia can further include a three-dimensional vector including a guideline indicating a trajectory of the virtual tool. The one or more processors can be configured to generate interactive elements for interacting with the smart headset and displaying the interactive elements superimposed within the environment.

In some implementations, the one or more processors can be configured to receive an instruction from an individual operating the smart headset. The one or more processors can be configured to lock the virtual tool superimposed within the environment. The virtual tool can be stationary at the desired location and the desired three-dimensional insertion angle as the smart headset changes positions within the environment. The instruction from the individual can be at least one of an eye movement, a gesture, an auditory pattern, a movement pattern, haptic feedback, a biometric input, intangible feedback, or a preconfigured interaction.

In some implementations, the visual indicia can include concentric circles indicating thresholds of the desired three-dimensional insertion angle of the surgical tool. The concentric circles can include a first set of concentric circles indicating the orientation of the surgical tool at the desired location based on the desired three-dimensional insertion angle and a second set of concentric circles indicating a live orientation of the surgical tool. The one or more processors can be calibrated to the surgical tool to indicate the live orientation of the surgical tool. The environmental data can include orientation data of the surgical tool. The one or more processors can continually collect the environmental data in real-time.

In some implementations, in response to continually collecting the environmental data, the one or more processors can be configured to automatically update, in real-time, the at least one graphical element superimposed within the environment. The one or more processors can include a gyroscope, and generating and displaying the at least one graphical element can be based on continually collecting, by the gyroscope in real-time, orientation data of the smart headset. In response to continually collecting the orientation data of the smart headset, the one or more processors can be configured to automatically update, in real-time, the at least one graphical element superimposed within the environment.

Some implementations relate to a method for orienting a surgical tool at a desired three-dimensional insertion angle at a desired location within an environment for use in installing a medical device by using and displaying at least one graphical element. The processing circuits can be configured to initiate a smart headset to be calibrated to the environment so that the position of the smart headset is known relative to the environment when the smart headset moves in the environment. The processing circuits can be configured to receive, by the smart headset from an electronic device communicatively coupled to the smart headset, environmental data indicating the position of the surgical tool within the environment. The processing circuits can be configured to receive, by the smart headset from the electronic device, the desired three-dimensional insertion angle. The processing circuits can be configured to generate, by the smart headset, at least one graphical element including visual indicia for orienting the surgical tool at the desired three-dimensional insertion angle at the desired location. The processing circuits can be configured to display, by the smart headset, the at least one graphical element superimposed within the environment.

Some implementations relate to a method for orienting a surgical tool at a desired three-dimensional insertion angle at a desired location within an environment for use in installing a medical device by using and displaying at least one graphical element. The processing circuits can be configured to determine the desired three-dimensional insertion angle of the surgical tool based on an orientation of the surgical tool. The processing circuits can be configured to collect environmental data of the surgical tool within the environment. The processing circuits can be configured to generate at least one graphical element including visual indicia for orienting the surgical tool at the desired location based on the desired three-dimensional insertion angle. The processing circuits can be configured to display, on a smart headset communicatively coupled to the one or more processors, the at least one graphical element superimposed within the environment.

Some implementations relate to a method for orienting a surgical tool at a desired three-dimensional insertion angle at a desired location within an environment for use in installing a medical device by using and displaying at least one graphical element. The processing circuits can be configured to initiate a smart headset to be calibrated to the environment so that the smart headset knows its position relative to the environment when the smart headset moves in the environment. The processing circuits can be configured to collect, by the smart headset, environmental data of the surgical tool within the environment using physical elements or fiducial markers or geometric shapes of the surgical tool that can be located at the desired location. The processing circuits can be configured to calculate, by the smart headset, an orientation of the surgical tool based on collecting the physical elements or fiducial markers or geometric shapes of the surgical tool. The processing circuits can be configured to receive, by the smart headset, the desired three-dimensional insertion angle. The processing circuits can be configured to determine the position of the desired three-dimensional insertion angle at the desired location. The processing circuits can be configured to generate, by the smart headset, the at least one graphical element including visual indicia for orienting the surgical tool at the desired three-dimensional insertion angle at the desired location. The processing circuits can be configured to display, by the smart headset, the at least one graphical element superimposed within the environment.

Some implementations relate to a method for orienting a surgical tool at a desired three-dimensional insertion angle at a desired location within an environment for use in installing a medical device by using and displaying at least one graphical element. The processing circuits can be configured to initiate a smart headset to be calibrated to the environment so that the smart headset knows its position relative to the environment when the smart headset moves in the environment. The processing circuits can be configured to collect, by the smart headset, environmental data of the surgical tool within the environment. The environmental data can include at least one of a gravitational vector and a two-dimensional plane relative to a portion of a body. The processing circuits can be configured to calculate, by the smart headset, an orientation of the surgical tool based on collecting the physical elements or fiducial markers or geometric shapes of the surgical tool. The processing circuits can be configured to receive, by the smart headset, the desired three-dimensional insertion angle. The processing circuits can be configured to determine the position of the desired three-dimensional insertion angle at the desired location. The processing circuits can be configured to generate, by the smart headset, the at least one graphical element including visual indicia for orienting the surgical tool at the desired three-dimensional insertion angle at the desired location. The processing circuits can be configured to display, by the smart headset, the at least one graphical element superimposed within the environment.

Some implementations relate to a system for orienting a tool at a desired location within an environment. The system can include an electronic device and a smart headset including a transparent display and communicatively coupled to the electronic device. The smart headset can be configured to initiate a smart headset to be calibrated to the environment so that the smart headset knows its position relative to the environment when the smart headset moves in the environment. The smart headset can be configured to receive, from the electronic device communicatively coupled to the smart headset, environmental data indicating the position of the surgical tool within the environment. The smart headset can be configured to receive, from the electronic device, the desired three-dimensional insertion angle. The smart headset can be configured to generate at least one graphical element including visual indicia for orienting the surgical tool at the desired three-dimensional insertion angle at the desired location. The smart headset can be configured to display the at least one graphical element superimposed within the environment.

Some implementations relate to a system for orienting a tool at a desired location within an environment. The system can include a smart headset including a transparent display and a processing circuit communicatively coupled to the smart headset. The processing circuits can be configured to determine a desired three-dimensional insertion angle of the surgical tool based on an orientation of the surgical tool. The processing circuits can be configured to collect environmental data of the surgical tool within the environment. The processing circuits can be configured to generate at least one graphical element including visual indicia for orienting the surgical tool at the desired location based on the desired three-dimensional insertion angle. The processing circuits can be configured to display, on a smart headset communicatively coupled to the one or more processors, the at least one graphical element superimposed within the environment.

Some implementations relate to a smart headset for orienting a tool at a desired location within an environment. The system can include a transparent display, a plurality of sensor devices, and one or more processors. The one or more processors can be configured to initiate the smart headset to be calibrated to the environment so that the smart headset knows its position relative to the environment when the smart headset moves in the environment. The one or more processors can be configured to collect, via the plurality of sensor devices, environmental data of the surgical tool within the environment using physical elements or fiducial markers or geometric shapes of the surgical tool that can be located at the desired location. The one or more processors can be configured to calculate an orientation of the surgical tool based on collecting the physical elements or fiducial markers or geometric shapes of the surgical tool. The one or more processors can be configured to receive the desired three-dimensional insertion angle. The one or more processors can be configured to determine the position of the desired three-dimensional insertion angle at the desired location. The one or more processors can be configured to generate at least one graphical element including visual indicia for orienting the surgical tool at the desired three-dimensional insertion angle at the desired location. The one or more processors can be configured to display, via the transparent display, the at least one graphical element superimposed within the environment.

### BRIEF DESCRIPTION OF THE DRAWINGS

For a more complete understanding of various present implementations and the advantages thereof, reference is now made to the following brief description, taken in connection with the accompanying drawings, appendices, and detailed description, wherein like reference numerals represent like parts, and in which:

FIG. 1 illustrates definitions of a sagittal plane, a frontal plane, and a axial plane relative to a patient's body;

FIG. 2A illustrates a cross-sectional, axial view of a vertebra having pedicle screws installed in respective pilot holes;

FIG. 2B illustrates an example lateral view of a vertebra for installing pedicle screws;

FIG. 2C illustrates an example posterior view of a vertebra for installing pedicle screws;

FIG. 3A presents a schematic diagram of an apparatus, which may be referred to as a medical alignment device, used in accordance with an embodiment to define and verify a three-dimensional alignment angle, which may also be referred to as an insertion angle, for use in installing devices, objects, hardware, and the like at a desired alignment angle;

FIG. 3B illustrates a schematic diagram of an axial view of a vertebra for defining an alignment or insertion angle for a pilot hole in the vertebra in this plane;

FIG. 4A illustrates a schematic side view of a medical operation system used in some implementations for defining the sagittal angle of a pilot hole to be made in a vertebra;

FIG. 4B illustrates a schematic front view of a medical operation system used in some implementations for defining the sagittal angle of a vertebra;

FIG. 5A illustrates an example flowchart for a method of determining an orientation of an instrument for inserting a medical device in a bone, in accordance with one or more implementations of the present disclosure;

FIGS. 5B, 5C, and 5D illustrate example flowcharts for methods for indicating the sagittal angle, transverse angle, and coronal angle, respectively, in accordance with one or more implementations of the present disclosure;

FIGS. 6A-6D illustrate example user interfaces for a computer-implemented program to perform the methods shown in FIGS. 5A-5D, wherein FIG. 6A illustrates an interface for selecting vertebra of a patient, FIG. 6B illustrates aligning the longitudinal axis of the apparatus with the sagittal plane, FIG. 6C illustrates defining a pedicle screw's position and its sagittal angle, and FIG. 6D illustrates generating an angle-indicative line for showing the angle between the longitudinal axis of the apparatus and the sagittal plane;

FIG. 7 illustrates an example of aligning the apparatus or medical alignment device;

FIG. 8 presents a schematic diagram of a system used in accordance with an embodiment to define and verify an insertion angle for a pilot hole in a vertebra;

FIG. 9 illustrates an example flowchart for a method of determining and displaying an orientation of an instrument for inserting a medical device in a bone, using an augmented reality device, in accordance with one or more implementations of the present disclosure;

FIG. 10 illustrates the system of FIG. 8 in use to assist with inserting a medical device in a bone;

FIG. 11 illustrates an augmented reality display presented by the system of FIG. 8 showing an orientation angle for an instrument for inserting a medical device in a bone;

FIG. 12 illustrates a virtual representation presented by the system, such as the medical alignment device or electronic device of FIG. 8, showing an axial view of a vertebra with a proposed alignment position of a pedicle screw shown that includes an insertion point and alignment angle for insertion or installation of the medical device into the bone or vertebra in this plane;

FIGS. 13A and 13B illustrate a virtual representation showing an orthogonal, lateral view of the vertebra and pedicle screw as set in the plane of FIG. 12, with the user able to establish the insertion location and alignment angle of the pedicle screw to be set in this plane so that the system, such as a medical alignment device, now has enough information as to the location of the pedicle screw in two orthogonal planes to determine a three-dimensional alignment angle for the installation of the pedicle screw in this vertebra;

FIG. 14 illustrates an example application of the aligning method presented in FIG. 5A in which the medical device is not properly angled for insertion into the bone;

FIG. 15 illustrates an example application of the aligning method presented in FIG. 5A in which the medical device is not properly angled for insertion into the bone, yet is more properly aligned than it was in FIG. 14;

FIG. 16 illustrates an example application of the aligning method presented in FIG. 5A in which the medical device is properly angled for insertion into the bone;

FIG. 17 illustrates the example applications shown in FIGS. 14-16 in operation on a smartphone;

FIG. 18 illustrates a user interface of the device of FIG. 3A in operation when selecting different views of a bone;

FIG. 19 illustrates a graphical user interface (GUI) of an orientation calibration system when the medical alignment device is properly oriented;

FIG. 20 illustrates an operation of using an orientation calibration system to calibrate an imaging source;

FIG. 21 illustrates a GUI of an orientation calibration system when the medical alignment device is out of the proper orientation;

FIG. 22 illustrates an operation of using an orientation calibration system to capture a reference image from an imaging source;

FIG. 23 is a flowchart showing an example of an orientation calibration process;

FIG. 24 illustrates a schematic diagram of a transverse view of a vertebra for defining an alignment or insertion angle for a pilot hole in the vertebra in this plane;

FIG. 25 illustrates a schematic diagram of a lateral view of the vertebra for defining the alignment or insertion angle for the pilot hole in the vertebra as shown in FIG. 24; and

FIG. 26 illustrates an example flowchart for a method of determining an orientation of an instrument for inserting a medical device in a bone by rotating the orientation about an insertion point.

FIG. 27 illustrates a schematic diagram of a lateral view of vertebral bodies for defining the installation of a medical device between the vertebral bodies.

FIG. 28 illustrates an example flowchart for a method for determining orientation of an instrument for positioning a medical device in a body.

FIG. 29 illustrates a surgical tool, an electronic device, and a smart headset in an environment, according to example implementations.

FIG. 30 illustrates a surgical tool and an electronic device, according to example implementations.

FIG. 31 illustrates a surgical tool, multiple electronic devices, and a smart headset in an environment, according to example implementations.

FIG. 32 illustrates a surgical tool, an electronic device, and a smart headset in an environment, according to example implementations.

FIGS. 33-35 illustrate various views of the smart headset of FIGS. 29-32, according to example implementations.

FIGS. 36-38 illustrate various views of the smart headset of FIGS. 29-32, according to example implementations.

FIG. 39 illustrates an example flowchart of a method for orienting a surgical tool at a desired three-dimensional insertion angle at a desired location within an environment, according to example implementations.

FIG. 40 illustrates another example flowchart of a method for orienting a surgical tool at a desired three-dimensional insertion angle at a desired location within an environment, according to example implementations.

FIG. 41 illustrates another example flowchart of a method for orienting a surgical tool at a desired three-dimensional insertion angle at a desired location within an environment, according to example implementations.

FIG. 42 is a block diagram of an example implementation of a smart headset, according to example implementations.

FIG. 43 is a block diagram illustrating an example computing system suitable for use in the various implementations described herein.

It will be recognized that some or all of the figures are schematic representations for purposes of illustration. The figures are provided for the purpose of illustrating one or more implementations with the explicit understanding that they will not be used to limit the scope or the meaning of the claims.

### DETAILED DESCRIPTION

In the following detailed description and the attached drawings and appendices, numerous specific details are set forth to provide a thorough understanding of the present disclosure. However, those skilled in the art will appreciate that the present disclosure may be practiced, in some instances, without such specific details. In other instances, well-known elements have been illustrated in schematic or block diagram form in order not to obscure the present disclosure in unnecessary detail. Additionally, for the most part, specific details, and the like, have been omitted inasmuch as such details are not considered necessary to obtain a complete understanding of the present disclosure, and are considered to be within the understanding of persons of ordinary skill in the relevant art.

The present implementations will now be described with reference to the following implementations. As is apparent by these descriptions, this implementation can be embodied in different forms and should not be construed as limited to the implementations set forth herein. Rather, these implementations are provided so that this disclosure will be thorough and complete, and will fully convey the scope of the implementations to those skilled in the art. For example, features illustrated with respect to one embodiment can be incorporated into other implementations, and features illustrated with respect to a particular embodiment may be deleted from that embodiment. In addition, numerous variations and additions to the implementations suggested herein will be apparent to those skilled in the art in light of the instant disclosure, which do not depart from the instant implementations. Hence, the following specification is intended to illustrate some particular implementations, and not to exhaustively specify all permutations, combinations and variations thereof.

Unless otherwise defined, all technical and scientific terms used herein have the same meaning as commonly understood by one of ordinary skill in the art to which this implementations belong. The terminology used in the description of the implementations herein is for the purpose of describing particular implementations only and is not intended to be limiting of the implementations.

All publications, patent applications, patents, and other references mentioned herein are incorporated by reference in their entirety.

Unless indicated otherwise, explicitly or by context, the following terms are used herein as set forth below.

As used in the description of the implementations and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

It is further noted that, unless indicated otherwise, all functions described herein may be performed in hardware or as software instructions for enabling a computer, radio or other device to perform predetermined operations, where the software instructions are embodied on a computer readable storage medium, such as RAM, a hard drive, flash memory or other type of computer readable storage medium known to a person of ordinary skill in the art. In certain implementations, the predetermined operations of the computer, radio or other device are performed by a processor such as a computer or an electronic data processor in accordance with code such as computer program code, software, firmware, and, in some implementations, integrated circuitry that is coded to perform such functions. Furthermore, it should be understood that various operations described herein as being performed by a user may be operations manually performed by the user, or may be automated processes performed either with or without instruction provided by the user.

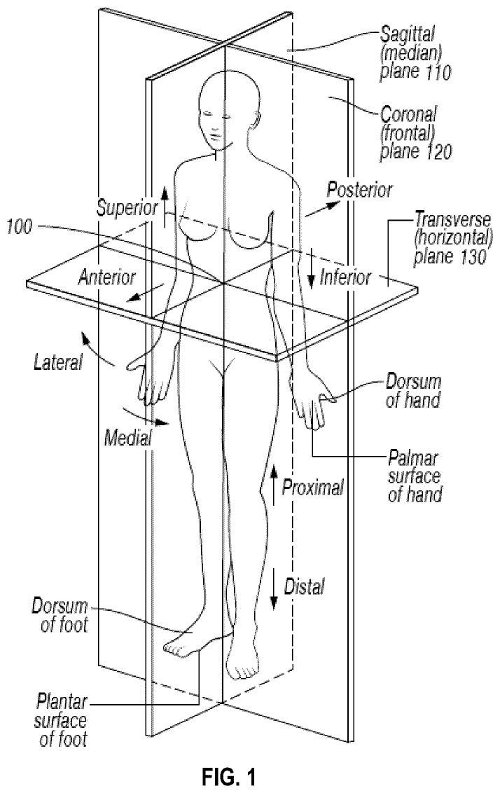

This disclosure describes an orientation calibration system for capturing a target image (also referred to as a reference image) and ensuring that the captured image is accurately captured, as well as methods of using and achieving the same. The orientation calibration system is illustrated herein in connection with FIGS. 1-18 as a medical alignment device operable to align a medical tool to a desired orientation relative to a patient (and a body part thereof). Although the current disclosure primarily describes orientation calibration system in connection with medical and diagnostic image applications, the orientation calibration system and related methods should not be understood to be limited to only medical type applications. On the contrary, such an orientation calibration system and related methods may be used for any of a variety of applications including, without limitation, for accurately capturing images at correct orientations, alignments, or angles for CAD drawings, construction drawings, maps, geology maps and formations, interior design, surgical navigation systems, three-dimensional printing applications, and the like.

The orientation calibration system ensures an accurate measurement of relative orientation between the medical alignment device and the patient. For example, the medical alignment device simulates an insertion angle relative to a reference image, such as a CT scan or other scan of a bone of the patient. The orientation calibration avoids a mistaken reading of the relative angle as measured by the orientation sensor between the medical alignment device and the reference image, and thus enabling accurate subsequent alignment indications.

At a high level, the orientation calibration system is applicable to both the medical alignment device and an image provider, such as a display monitor showing or displaying a target image, such as a diagnostic image such as a CT or MRI scan. In some implementations, the medical alignment device includes a display and an orientation sensor. The display of the medical alignment device may include an indicator, such as a graphical indicator, that shows a present orientation of the medical alignment device relative to some orientation or known reference orientation. The reference orientation may be determined by aligning to a gravitational direction or the image provider, such as the monitor displaying an image. For example, the medical alignment device may be positioned and aligned to the image provider in the same plane. When capturing a copy of the reference image shown in the image provider, the medical alignment device can be oriented to be parallel to the image provider and have one longitudinal axis aligned with the gravitational direction (or forming a known angle relative to the gravitational direction). As such, the calibration allows the medical alignment device to ascertain subsequent increments of orientation to provide accurate readings.

FIG. 1 illustrates a sagittal or median plane 110, a frontal or coronal plane 120, and a horizontal or transverse plane 130 relative to a patient's body part 100 located at the intersection of the sagittal plane 110, the coronal plane 120, and the transverse plane 130. Each plane is orthogonal to each other such that if the position or orientation of an object, device, or medical hardware, such as a pedicle screw, is known in two of the orthogonal planes, the three-dimensional orientation angle of such item may be calculated or known. When discussing a vertebra (or other body parts) in the following disclosure, reference is made to the sagittal plane, coronal plane, and transverse plane. It should be understood that, when these planes are mentioned, they are not intended as a reference only to the specific sagittal, coronal, and transverse planes illustrated in FIG. 1, but rather, are intended as a reference to illustrate an orientation or location relative to the specific vertebra or body part being discussed.

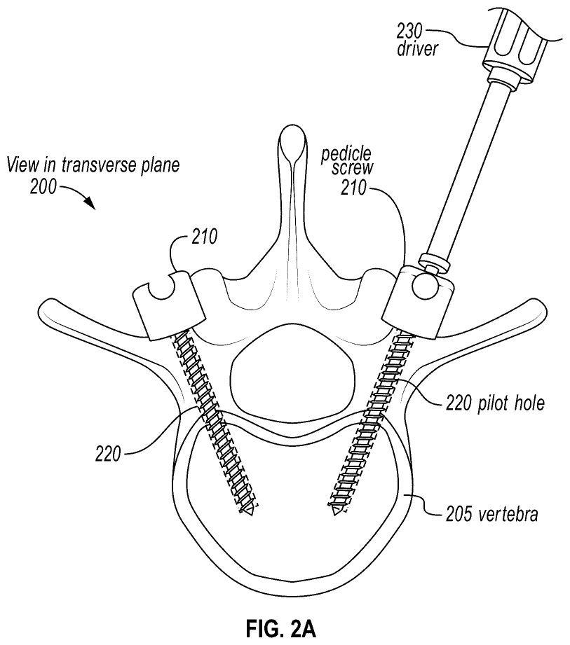

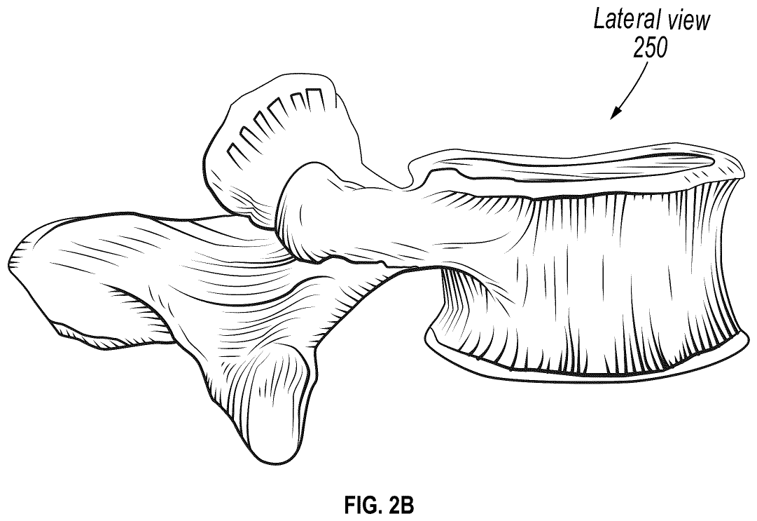

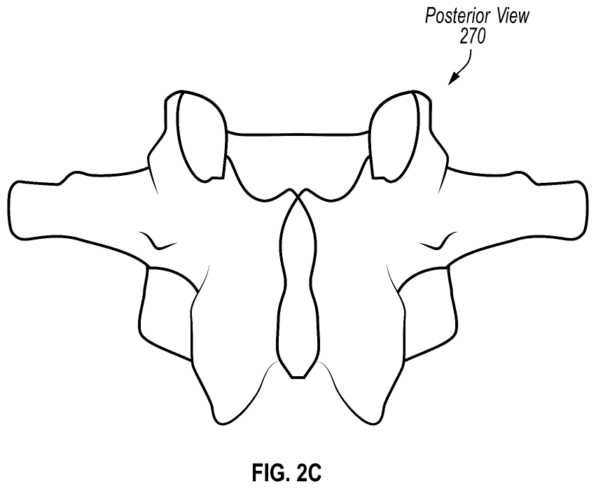

FIG. 2A illustrates a cross-sectional, axial view (may be referred to as a superior view) 200 of a vertebra 205 having pedicle screws 210 installed in respective pilot holes 220. A driver 230 may be used to screw the pedicle screws 210 positioned in pilot holes 220. Various shapes and types of pedicle screws 210 and driver 230 may be used. The pedicle screws 210 and driver 230 shown in FIG. 2A are for illustrative purpose only. A mating portion 252 of the driver 230, which may be referred to as a tool or a medical tool, may be provided to allow a medical alignment device in an attachment apparatus to “mate” or position adjacent such mating portion 252 to ensure that the driver 230 is installing the pedicle screw at a desired alignment angle, such as a three-dimensional alignment angle. FIG. 2B illustrates a lateral view (i.e., side view) 250 of a vertebra, which could be an orthogonal view of the vertebra 205 of FIG. 2A. FIG. 2C illustrates a posterior view 270 of a vertebra. The following discussion focuses on properly creating the pilot holes with a tool guided by the present disclosure.

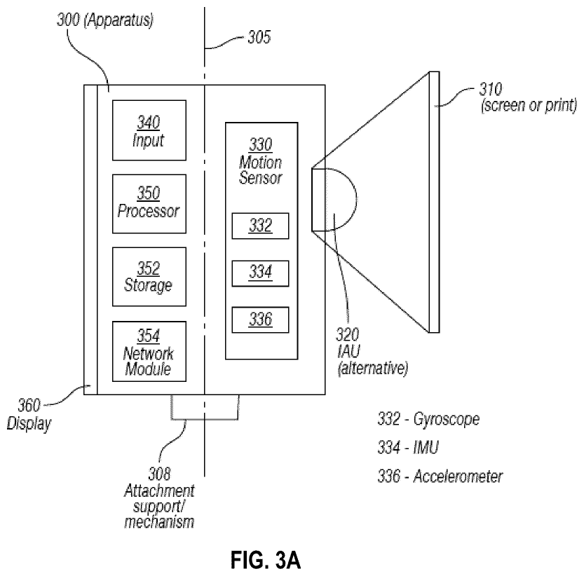

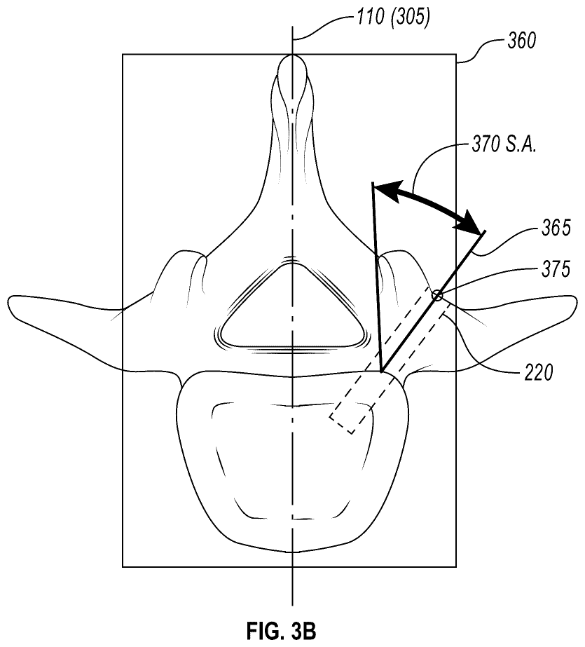

FIG. 3A presents a schematic diagram of an apparatus 300, which may be referred to as a medical alignment device or alignment device, used in accordance with an embodiment to define and verify an angle, such as a three-dimensional alignment angle, for use in installing devices, objects, hardware, and the like, such as to align a pilot hole, or tract, such as the pilot hole 220 of FIG. 2. The apparatus 300 has an axis 305 (such as, for example, a longitudinal axis) that is used in some implementations to align the apparatus 300 for image capture. The apparatus 300 includes an image acquisition unit 320 (or camera) for capturing an image 310 of the vertebra. In some implementations, the image 310 may be obtained by positioning the apparatus 300 and/or image acquisition unit 320 in parallel with the transverse, sagittal, or coronal plane to obtain an image of the vertebra. These images may be diagnostic images such as, for example, CT scans, MRI scans, X-rays, and the like of items of interest, such as a vertebra. In some implementations, an attachment support and/or mechanism 308 is used to align and/or secure the apparatus 300 to a tool that creates a pilot hole for example.

In some implementations, the image acquisition unit 320 can be a camera having sufficient field of view in display 360 to properly align the axis 305 of the apparatus 300 with a desired plane. In some implementations, the axis 305 is representative of a vertical line centered laterally with respect to the image being captured. For example, if the desired image is intended to capture the vertebra from a cross sectional, axial view (e.g., see FIG. 2A), the axis 305 is aligned with the sagittal plane (i.e., the plane that is sagittal to the vertebra) and the image acquisition unit 320 is positioned parallel to the transverse plane to capture the top-down view of the vertebra shown in FIG. 2A. If the desired image is intended to capture the vertebra from a side view (e.g., a lateral image of the vertebra, see FIG. 2B), the axis 305 is aligned with the transverse plane (i.e., the plane that is transverse to the vertebra) and the image acquisition unit 320 is positioned parallel to the sagittal plane. If the desired image is intended to capture the vertebra from a posterior or anterior view (see, for example, FIG. 2C), the axis 305 is aligned with the sagittal plane and the image acquisition unit 320 is positioned parallel to the coronal plane.

In some implementations, the image 310 may be a processed diagnostic image, e.g., an image displayed on a screen, a film, or a printed photograph. In other implementations, the image acquisition unit 320 can directly use an image taken from an external machine (not illustrated), such as a radiograph, computed tomography (CT) scanner, or a magnetic resonance imaging (MRI) machine.

The orientation apparatus 330 is operable to detect changes in movement, orientation, and position. In some implementations, the orientation apparatus 330 includes at least one of a gyroscope 332, an inertial measurement unit 334, and an accelerometer 336, in other implementations it may only include the gyroscope 332 with three axes of rotation to be able to determine a three-dimensional orientation of the apparatus 300. The gyroscope 332 is operable to measure at least one axis of rotation, for example, the axis parallel to the intersection of the sagittal plane and the coronal plane. In other implementations, the gyroscope 332 includes more than one sensing axes of rotation, such as three axes of rotation, for detecting orientation and changes in orientation. The inertial measurement unit 334 can detect changes of position in one or more directions in, for example, a cardinal coordinate system. The accelerometer 336 can detect changes of speeds in one or more directions in, for example, a cardinal coordinate system. In some implementations, data from all components of the orientation apparatus 330 are used to calculate the continuous, dynamic changes in orientation and position.

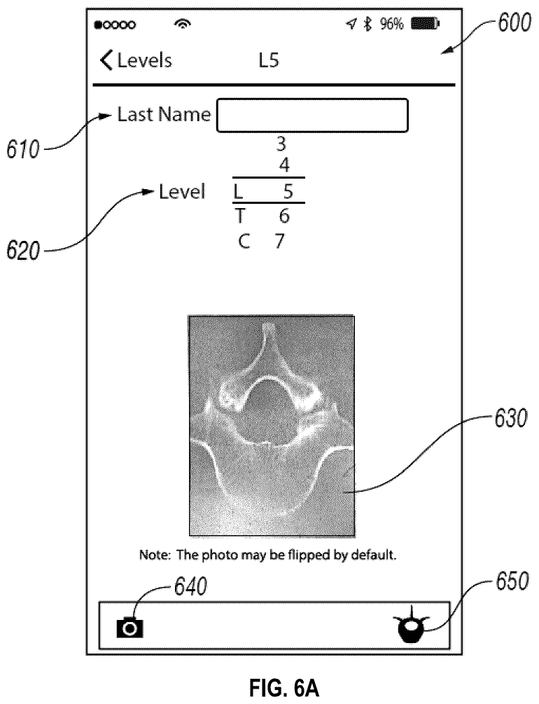

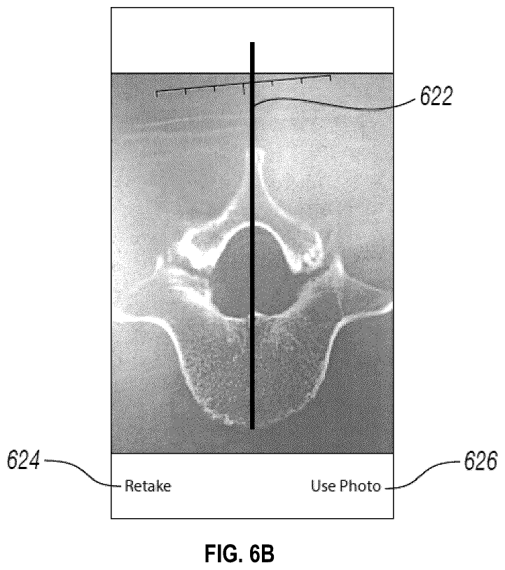

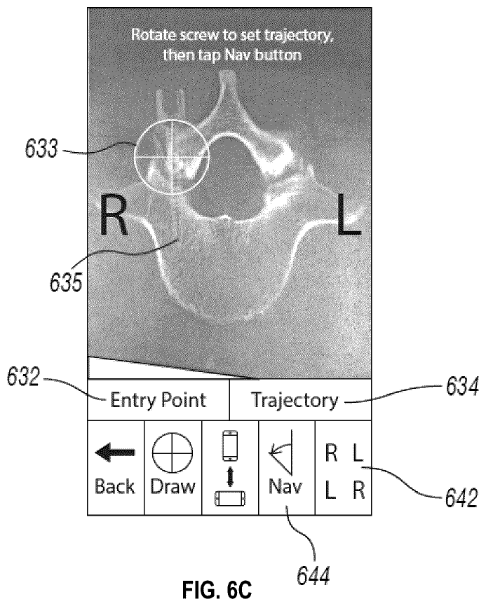

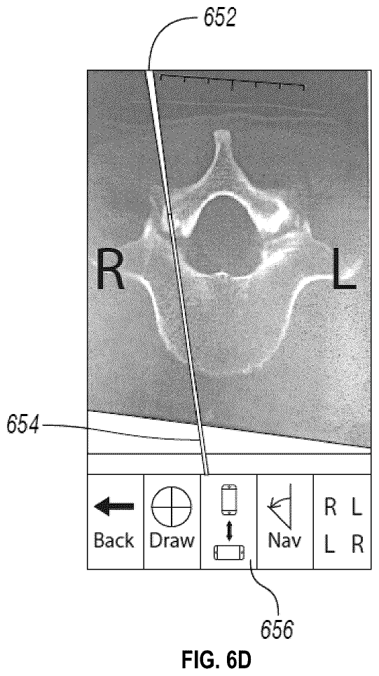

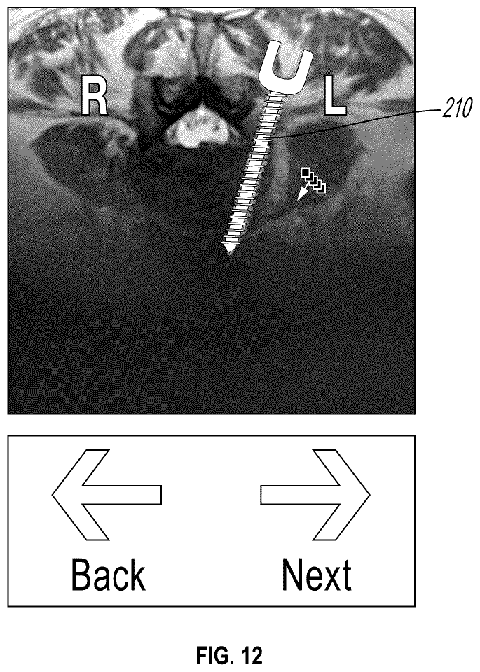

The apparatus 300 further includes, in some implementations, an input component 340 that is operable to receive user input, such as through a keypad or touchscreen, to receive a device, such as a pedicle screw to be installed in a vertebra, insertion location and the desired angle representing an insertion direction of the pedicle screw. An example illustration of the user input component 340 is presented in accordance with FIGS. 6A-6D, as well as FIGS. 12, 13A, 13B, and 18. In some implementations, the input component 340 can include a multi-touch screen, a computer mouse, a keyboard, a touch sensitive pad, or any other input device.

In some implementations, the apparatus 300 further includes a processor 350. The processor 350 can be any processing unit capable of basic computation and capable of executing a program, software, firmware, or any application commonly known in the art of computer science. As to be explained, the processor 350 is operable to generate a three-dimensional alignment angle based on alignment inputs from to views orthogonal to one another, and to output an angle-indicative line representing the orientation of a device, such as a pedicle screw, pilot hole, etc. on the display showing a diagnostic image where the device, such as a pedicle screw, is to be installed. In some implementations, the angle-indicative line provides a notation that the orientation of the apparatus 300 approximately forms the desired angle. The angle-indicative line is not limited to showing sagittal angles, but also angles in different planes, such as, for example, the coronal plane or the transverse plane.

The apparatus 300 may, in some implementations, further include a memory storage unit 352 and network module 354. The memory storage unit 352 can be a hard drive, random access memory, solid-state memory, flash memory, or any other storage device. Memory storage unit 352 saves data related to at least an operating system, application, and patient profiles. The network module 354 allows the apparatus 300 to communicate with external equipment as well as communication networks.

In some implementations, the apparatus 300 further includes a display 360 (e.g., field of view). In some implementations, the display 360 is a liquid crystal display that also serves as an input using a multi-touch screen. In some implementations, the display 360 shows the angle-indicative line to a user and provides a notification when the apparatus is approximately aligned with the predefined desired angle, as determined by the gyroscope 332 or the orientation apparatus 330. For example, the notification can include a highlighted line that notifies the user the axis 305 has reached the desired angle, or is within an acceptable range of the desired angle. The apparatus 300 may provide any number of notifications to a user, including visual, auditory, and tactile, such as, for example, vibrations. The apparatus 300 will include a speaker as well as a device to impart vibrations to a user to alert or notify a user.

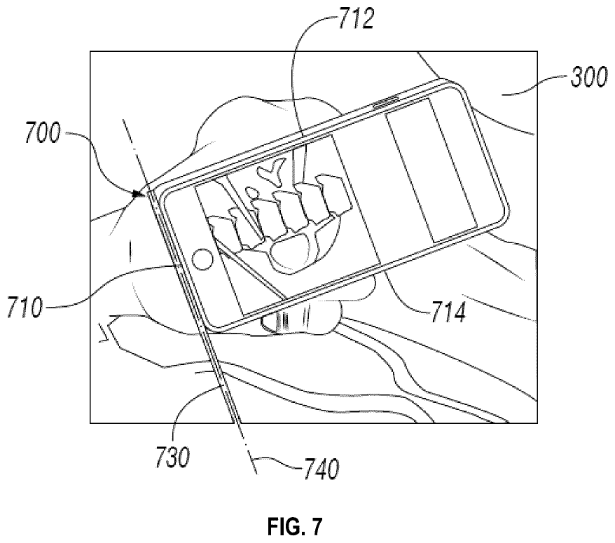

Referring briefly to FIG. 7, in some implementations, the apparatus 300 (i.e., the medical alignment device) further includes an attachment support or mechanism 700 (also 308 of FIG. 3A) that allows the medical alignment device or apparatus 300 to be attached or provided adjacent to a tool, medical hardware, or equipment (i.e. a medical tool 730). The attachment apparatus 700 may be made of plastic, stainless steel, titanium, or any other material. The attachment apparatus 700 couples to the medical alignment device or apparatus 300 to the tool 730 by, for example, providing a casing that is attached to the medical alignment device 300 and is configured to connect to or about the medical tool 730, for example, by aligning a first surface 710 of the medical alignment device 300 to the attachment apparatus 700 and thus to the medical tool 730. For example, the attachment apparatus 700 may be aligned to a longitudinal axis 740 of the medical tool 730. As such, orientation sensors in the medical alignment device 300 are properly aligned with the longitudinal axis 740.

In other implementations, a second surface 712 and a third surface 714 of the medical alignment device 300 may be used to secure and/or align the medical alignment device 300 to the attachment apparatus 700. In some implementations, the attachment apparatus 700 may include a magnetic attachment apparatus for coupling the medical alignment device 300 to the tool 730 or to the attachment apparatus 700. The attachment apparatus 700 allows the medical alignment device 300 to provide real-time measurement and display of the orientation of the attached or aligned medical tool 730.

Returning to FIG. 3B, a schematic diagram of an axial view of a vertebra defining an alignment or insertion angle for a pilot hole in the vertebra in this plane for insertion or installation of a pedicle screw is provided. This view or diagnostic image of the vertebra may be electronically transmitted to the medical alignment device 300, or the view or image may be captured from a monitor or display of a diagnostic image using the image acquisition unit 320 of the medical alignment device 300 (sometimes referred to as apparatus 300). A sagittal angle 370 may be defined for the pilot hole 220 in the vertebra 205 that starts at the initial position 375, which may be referred to as the insertion location. The display 360 shows the field of view of the view captured by the image acquisition unit 320, assuming that was how the image was acquired, and allows a user to align the axis 305 of the apparatus 300 with the desired plane (e.g., the sagittal plane). In the embodiment shown in FIG. 3B, the sagittal angle 370 is the angle between the central axis 365 of the pilot hole 220 and the sagittal plane.

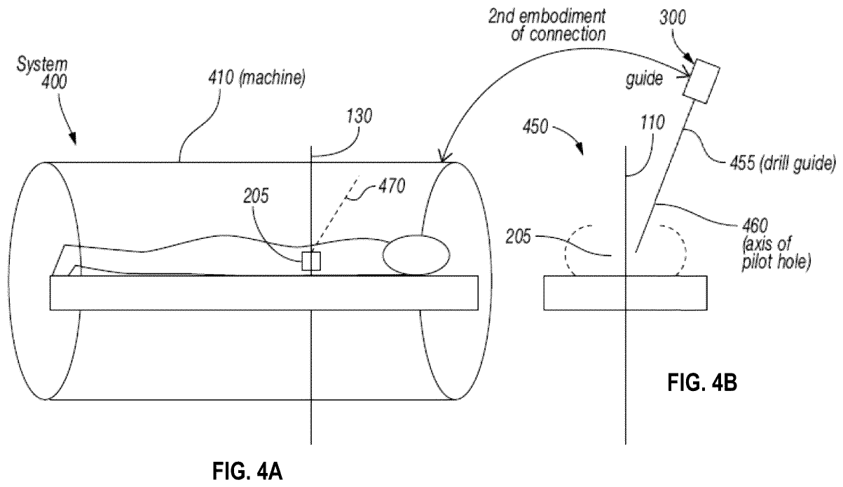

FIG. 4A illustrates a schematic side view of a medical operation system 400 used in some implementations for defining the sagittal angle 370 of a pilot hole to be made in a vertebra which may be used in some implementations for defining the sagittal angle 370 of the vertebra shown in FIGS. 3A and 3B. The medical operation system 400 includes a machine 410 for capturing a cross-sectional view of the vertebra 205. The machine 410 may be, for example, a CT scanner or MRI machine. The patient exits the machine 410 after the image is taken, as shown in FIG. 4B.

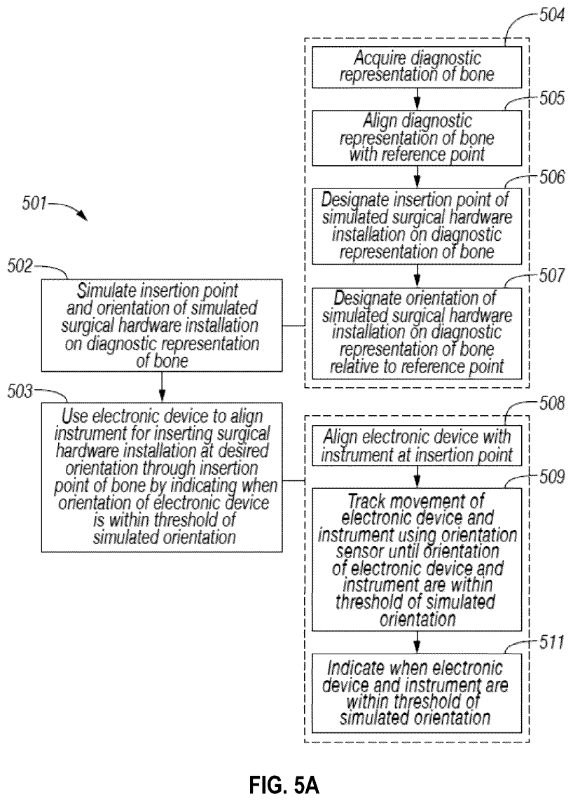

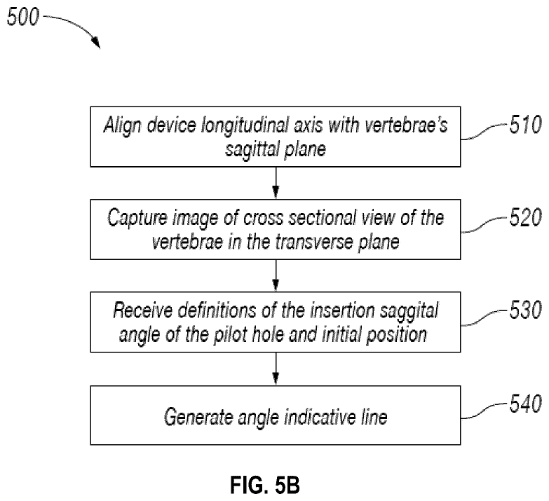

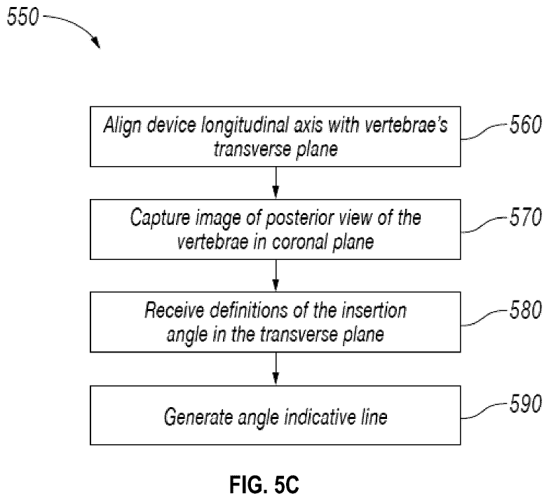

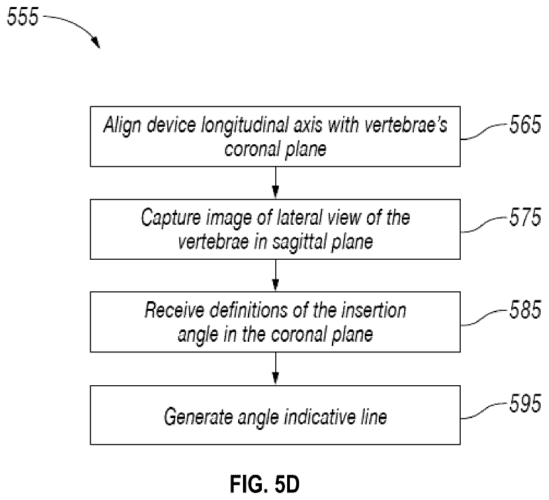

FIG. 4B illustrates a schematic front view 450 of the medical operation system 400 taken in the transverse plane for defining the sagittal angle 370 of the vertebra 205. The front view axis 460 (and correspondingly, the side view axis 470) of the pilot hole should be precisely defined for the drilling guide 455. In some implementations, the apparatus 300 may be attached to the drilling guide 455 with the attachment support/mechanism 308. Defining and verifying the sagittal angle 370 may be performed at the apparatus 300, as explained in connection with the method illustrated in FIG. 5B.

First, however, an example method of determining an orientation of an instrument for inserting a medical device in a bone is now described with reference to the flowchart 501 of FIG. 5A. A diagnostic image is obtained at the apparatus 300 and displayed. An insertion point and a desired orientation of a simulated surgical hardware installation are simulated and displayed on a diagnostic representation of a bone at block 502 and the desired alignment orientation is stored. Proceeding to block 503, the apparatus or medical alignment device 300 with orientation sensor, such as gyroscope 332, is used to align a tool, such as a medical tool, drill or the like for inserting or installing the surgical hardware at the desired alignment orientation from block 502 and through the insertion point of the bone by indicating when an orientation of the medical alignment device 300 is within a threshold of the simulated orientation with the desired alignment angle.

Simulating the insertion point and the orientation of the simulated surgical hardware installation on the diagnostic representation of the bone includes acquiring the diagnostic representation of the bone at block 504, aligning the diagnostic representation of the bone with a reference point at block 505, designating the insertion point of the simulated surgical hardware installation on the diagnostic representation of the bone at block 506, and designating the orientation of the simulated surgical hardware installation on the diagnostic representation of the bone relative to the reference point at block 507.

If block 502 is repeated using a second diagnostic representation of the bone that is orthogonal to the first diagnostic representation, the same steps 504 through 507 may be repeated on the second diagnostic representation with the location of the simulated surgical hardware constrained to the selections or settings made when the insertion point and orientation were selected in the first diagnostic representation. Once this is done, a three-dimensional alignment angle may be calculated or determined. This may be done by the apparatus or medical alignment device 300.

Using the electronic device, which may be the apparatus or medical alignment device 300, to align the instrument or tool for inserting the surgical hardware installation at the desired orientation through the insertion point includes aligning the electronic device with the instrument or tool at the insertion point in block 508, tracking movement or orientation of the electronic device and the instrument or tool using an orientation sensor, such as gyroscope 332, of the electronic device until the orientation of the electronic device and the instrument are within the threshold of the simulated orientation at block 509, and indicating when the electronic device and the instrument are within the threshold of the simulated orientation at block 511. The indication may be visual, auditory, or tactile. The orientation of the electronic device, and hence the alignment of the instrument or tool, may be a two-dimensional alignment angle, in certain implementations, or a three-dimensional alignment angle. FIG. 7 illustrates an example application of the alignment of block 508.

FIGS. 5B, 5C, and 5D illustrate example flowcharts for methods for indicating or determining a desired alignment angle, which also may be referred to as an insertion angle, in the: (i) sagittal plane, which may be referred to as the sagittal angle, (ii) the transverse plane, which may be referred to as the transverse angle, and (iii) the coronal plane, which may be referred to as the coronal angle, respectively, in accordance with one or more implementations of the present disclosure. Each of these methods may be thought of as generating or determining a two-dimensional alignment angle in their respective plane.

FIG. 5B illustrates an example flowchart 500 of a method for indicating the sagittal angle 370. The method of the flowchart 500 is for verifying any insertion angle 370 of the pilot hole 220 in the sagittal plane 110 for receiving a pedicle screw 210 in the vertebra 205. At 510, the axis 305 of the apparatus 300 is aligned or is oriented with the sagittal plane of an image of the vertebra, in this embodiment. In some implementations, a user may hold the apparatus 300 and rotate the apparatus 300 to match a marking indicating the axis 305 with features of the vertebra 205 that indicate the sagittal plane. In some implementations, the marking may be displayed on the screen as the user aligns the device. In other implementations, the image of the vertebra (or other desired object or bone) is a diagnostic image that is displayed on the apparatus 300, which may be a medical alignment device 300, and is already oriented in some manner to the sagittal plane.

At 520, the image of the cross-sectional view is captured in the transverse plane. In some implementations, the apparatus 300 includes a smart phone, a tablet computer, a laptop computer, or any portable computational device including those that include a camera for capturing a representation of the cross-sectional view of the vertebra 205. In other implementations, the image of the vertebra 205 may be sent or transmitted to the apparatus 300 via a wired or wireless connection to be displayed on the apparatus 300 such that no physical representation (e.g., films, photos, monitors) may be needed for this step.

At 530, definitions of the insertion sagittal angle 370 of the pilot hole 220 and the initial position 375, also referred to as the insertion location, of the pilot hole 220 are provided or specified by a user. This input operation may be performed using various input devices of the apparatus 300, including a computer mouse, a keyboard, a touchscreen, or the like. In some implementations, a multi-touch screen (e.g., the display 360) is used for both displaying the image and receiving the definition input from a user. Example illustrations of this input are provided in FIGS. 6A-6D, where the insertion location or initial position 375 of the pilot hole 220 for the installation of a pedicle screw are established by locating (or simulating) graphically the insertion location on the displayed diagnostic image, and the applicable alignment angle for the displayed plane may defined by moving or locating (or simulating) the desired position of the alignment angle of the pilot hole/pedicle screw.

At 540, an angle-indicative line is generated by a processor and displayed on the display 360 along with the diagnostic image. The angle-indicative line can rotate in response to the apparatus 300 rotation and provides a notification when the orientation or position of the apparatus 300 approximately forms the insertion sagittal angle 370 between the apparatus 300 longitudinal axis 305 and the sagittal plane. In some implementations, the angle-indicative line is a rotating line generated in the display 360 that allows a user to monitor the change of orientation of the apparatus 300. The orientation monitoring is performed with an orientation apparatus 330. More specifically, in some implementations, a gyroscope 332 that includes at least one axis of rotation may provide the function of monitoring the orientation or position of apparatus 300 to generate the current orientation of the apparatus 300. This current orientation may be compared to the desired insertion angle (or alignment angle) discussed above in connection with 530 to determine whether or not alignment exists or the extent of alignment, and this may be compared or shown graphically.

The indicative line may generate notations in various forms, including a visual alert such as highlighting the angle-indicative line, an audio alert such as providing a continuous sound with variable frequency indicative of the proximity between the current angle and the desired angle, and a small vibration that allows the user to notice the angular change. It should be appreciated that any audio alert may be used, such as a single sound or series of sounds when the desired angle is reached. Likewise, a single vibration or a series of vibrations may be emitted when the desired angle is reached. In some implementations, the flowchart 500 illustrated in FIG. 5B may be applicable for generating indication angles in the transverse plane or the coronal plane for indicating a respective transverse angle or a coronal angle.

FIG. 5C illustrates a flowchart 550 of an implementation for indicating a transverse angle, which is an angle with respect to the transverse plane of the vertebra. The method of the flowchart 550 is for verifying any pedicle screw insertion angle in the transverse plane of the vertebra 205. At 560, the axis 305 of the apparatus 300 is aligned with the transverse plane. In some implementations, a user may hold the apparatus 300 and rotate the apparatus 300 to match a marking indicating the axis 305 with features of the vertebra 205 that indicate the transverse plane. In some implementations, the marking may be displayed on the screen as the user aligns the device.

At 570, an image of the posterior view is captured or provided in the coronal plane. In some implementations, the apparatus 300 includes a smart phone, a tablet computer, a laptop computer, or any portable computational device including those that include a camera for capturing a representation of the cross-sectional view of the vertebra 205. In other implementations, the image of the vertebra 205 may be sent to the apparatus 300 via a wired or wireless connection to be displayed on the apparatus 300 such that no physical representation (e.g., films, photos, monitors) may be needed for this step.

At 580, definitions of the insertion angle in the transverse plane 130, and the initial position 375 of the pilot hole are provided by a user, as similar to the sagittal angle defined at 530.

At 590, an angle-indicative line for the corresponding transverse angle is generated by a processor and displayed on the display 360. The angle-indicative line can rotate in response to the apparatus 300 rotation and provides a notification when the apparatus 300 approximately forms the insertion transverse angle, as defined in step 580, between the apparatus 300 longitudinal axis 305 and the transverse plane. In some implementations, the angle-indicative line is a rotating line generated in the display 360 that allows a user to monitor the change of orientation of the apparatus 300. The orientation monitoring is performed with an orientation apparatus 330. More specifically, in some implementations, a gyroscope 332 that includes at least one axis of rotation may provide the function of monitoring the orientation or position of the apparatus.

FIG. 5D illustrates a flowchart 555 of another implementation for indicating a coronal angle. The method of the flowchart 555 is for verifying any insertion angle of a pedicle screw 210 in the vertebra 205 in the coronal plane 120. At 565, the axis 305 of the apparatus 300 is aligned with the coronal plane. In some implementations, a user may hold the apparatus 300 and rotate the apparatus 300 to match a marking indicating the axis 305 with features of the vertebra 205 that indicate the coronal plane. In some implementations, the marking may be displayed on the screen as the user aligns the device.

At 575, the image of the lateral view is captured in the sagittal plane. In some embodiment, the apparatus 300 includes a smart phone, a tablet computer, a laptop computer, or any portable computational device including those that include a camera for capturing a representation of the posterior view of the vertebra 205. In other implementations, the image of the vertebra 205 may be sent to the apparatus 300 via a wired or wireless connection to be displayed on the apparatus 300 such that no physical representation (e.g., films, photos, monitors) may be needed for this step.

At 585, respective definitions of the insertion angle in the coronal plane 120, and the initial position 375 of the pilot hole are provided by a user, as similar to the sagittal angle defined at 530.

At 595, an angle-indicative line for one of the corresponding coronal angle is generated by a processor and displayed on the display 360. The angle-indicative line can rotate in response to the apparatus 300 orientation and provides a notification when the apparatus 300 approximately forms the insertion coronal angle between the apparatus 300 longitudinal axis 305 and the coronal plane. In some implementations, the angle-indicative line is a rotating line generated in the display 360 that allows a user to monitor the change of orientation of the apparatus 300. The orientation monitoring is performed with an orientation apparatus 330 of the apparatus 300. More specifically, in some implementations, a gyroscope 332 that includes at least one axis of rotation may provide the function of monitoring the apparatus's orientation or position.

FIGS. 6A-6D illustrate examples of user interfaces for controlling a computer implemented program to perform the methods shown in FIG. 5A-5D. FIG. 6A illustrates an interface 600 for selecting vertebra of a patient, FIG. 6B illustrates displaying a diagnostic image and aligning (or confirming the alignment) the axis 305 of the apparatus 300 with the sagittal plane of the image, FIG. 6C illustrates defining a pedicle screw's position, including its insertion location or entry point at the cross hair, and its sagittal angle 370 on the diagnostic image, and FIG. 6D illustrates generating an angle-indicative line for showing the angle between the longitudinal axis of the apparatus and the sagittal plane. In some implementations, the angle-indicative line may represent a virtual gear shift, pedicle probe, Jamshidi needle wherein the surgical tool is one of a gear shift probe, a pedicle probe, a Jamshidi needle, an awl, a tap, a screw inserter, a drill, or a syringe, or other instrument for aligning a pedicle screw or pilot hole. When the virtual gear shift or angle is properly aligned, the virtual gear shift may change colors, or may change length or width. The angle-indicative line can rotate or reorient in response to the apparatus 300 rotation or reorientation, and provides a notification when the apparatus 300 approximately forms the desired alignment angle in this view between the apparatus 300 longitudinal axis 305 and the desired alignment angle.

In FIG. 6A, the patient's profile may be selected or added by typing the last name of the patient in the window 610. The corresponding vertebra for the desired angle is selected in the window 620. The camera button 640 allows a user to take a picture of a diagnostic image of the actual vertebra or to receive such a diagnostic image. The diagnostic image or picture is shown in the window 630. The button 650 allows the user to move onto the next step. As previously discussed, the picture at the vertebra may be provided without use of the camera or camera button 640.

For example, by using a camera of a mobile device, a user can take a picture of an axial view (either CT or MRI) in the transverse plane 130, of the desired vertebral body 205. Use the line 622 to line up the vertebral body so that it is proximately vertical for aligning with the sagittal plane (or other desired plane), as shown in FIG. 6B. A retake button 624 allows the user to go back to the previous steps to retake the image to ensure the alignment is proper. The button 626 allows the user to select the current photo to be used in the following operations.

After selecting button 626, the user may be returned to the detail view as shown in FIG. 6C. The photo may, in some implementations, be automatically flipped to approximate its position during surgery. Button 642 may be selected to flip the orientation of the photo. For example, the RL button 642 can be used to flip the picture (and pedicle screw) depending on whether the surgeon is placing the screw while looking towards the patient's head (e.g., in the longitudinal axis toward the cephalad direction) or towards their feet (e.g., in the longitudinal axis toward the caudal or caudad direction).

The user next selects the optimal pedicle screw position by selecting the navigation button 644 to move the simulated pedicle screw to a desired location by moving a crosshairs 633 to the cortical entry point of the screw, for example, by tapping the entry point button 632 to confirm, and then tapping the trajectory button 634 and rotate the screw to its desired position 635. The crosshairs 633 specify the insertion location, such as the initial position 375 of FIG. 3B.

Tap the Nav button 644 and a virtual gear shift probe 652 (which may represent any tool or axis, such as a drill or pilot hole longitudinal axis) appears on the screen. The gear shift probe's orientation matches the orientation of the apparatus 300, which will include orientation circuitry, such as a gyroscope to determine the orientation of apparatus 300. In some implementations, once the angle of the gear shift probe 652 is about 20 degrees within the selected trajectory, the gear shift probe 652 will turn yellow, at 5 degrees, it will turn green, and when the alignment is within 1 degree of the target angle, a green line 654 will extend outward and the pedicle screw will disappear to signify that the apparatus 300 is properly aligned. In some implementations, the virtual gear shift probe 652 may be a Jamshidi needle or other surgical instrument.

In some implementations, the device or apparatus 300 can be placed in a sterile bag and then be placed against the gear shift probe (or Jamshidi needle) as it is being used to create the path for the pedicle screw. As provided herein, the apparatus 300 may be positioned in an attachment apparatus so that the apparatus 300 may be conveniently aligned or abutted with a tool, such as the gear shift probe, drill, and the like.

Some gear shift probes (or Jamshidi needles) may be too short to allow the device (apparatus 300) to be placed against them lengthwise. If this is the case, tap the 90-degree button 656 and the screen will be rotated so the short edge of the device can be placed against the gear shift probe (or Jamshidi needle).

Other implementations of the disclosed system and method are possible. For example, the apparatus 300 may also use a second or more views to define various angles not limited within the sagittal plane. For example and in accordance with the foregoing disclosure, images of the vertebra may be captured from two orthogonal planes, such as through superior, lateral, posterior, anterior views, and various combinations thereof, to provide multiple reference points so that three-dimensional representations of the alignment angles can be presented.

In addition, different mobile computer devices may be used or modified into, or as, the apparatus 300 by equipping corresponding image acquisition units, input terminals, and motion or orientation sensing units. In some implementations, the apparatus 300 may include a smart phone or another electronic device having a gyroscope. In addition, other motion or orientation sensors may be included such as the inertial measurement unit 334, and the accelerometers 336. The apparatus 300 may also be attached onto various medical devices or equipment for guiding insertion angles that require high precision and ease of use. In certain implementations, the apparatus 300 may be implanted using a smartphone such as, for example, an iPhone. Also, in some applications and implementations, the apparatus 300 may include one or more of an iPod Touch, iPad, Android phone, Android tablet, Windows Phone, Windows tablet, Blackberry phone, or other suitable electronic device. Also, in some applications, the mobile computer device or apparatus 300 may be an Apple TV in combination with an Apple TV remote, or a Nintendo Wii in combination with a Nintendo Wii remote, or other combinations of electronic devices. Indeed, the mobile computer device may be any combination of electronic devices where the orientation sensor (such as a gyroscope) is in one electronic device and the processor or processors are in another electronic device.

In some implementations, axis other than the device's longitudinal axis may be used. Axes can be defined by a portion of the device (e.g., an edge or surface of the device). More than one orientation apparatus 330 may be used at the same time, if desired. Surgical apparatus may include pedicle screws, gear shift probes, Jamshidi needles, instruments for percutaneous operations, syringes, medical implants, and other medical devices.

It should be appreciated that the various methods and techniques described above may be utilized with a virtual reality or augmented reality device, either on its own or in conjunction with another electronic device such as a smartphone or computer. The determination of the insertion point or pilot hole and the proper angle for the surgical tool used to attach or install the pedicle screw or other medical device may proceed in any of the fashions as described above, and then the virtual reality or augmented reality device may be used to display the proper insertion point or pilot hole and proper angle for the surgical tool to a physician.

In the case of a virtual reality device, the simulation of a tool or axis at a desired three-dimensional alignment angle or other alignment angle may be displayed to the surgeon or user in an immersive three-dimensional fashion so that the surgeon can view the bone or tools used in a procedure as it will appear during a surgery. In addition, the planning of the insertion point or pilot hole and the proper angle for the surgical tool may be conducted with the aid of the virtual reality device.

In the case of an augmented reality device, during the actual surgery, virtual visual indicia may be displayed superimposed over the real bone, illustrating to the physician precisely where to insert the surgical tool and at precisely which angle the surgical tool should be inserted and operated.

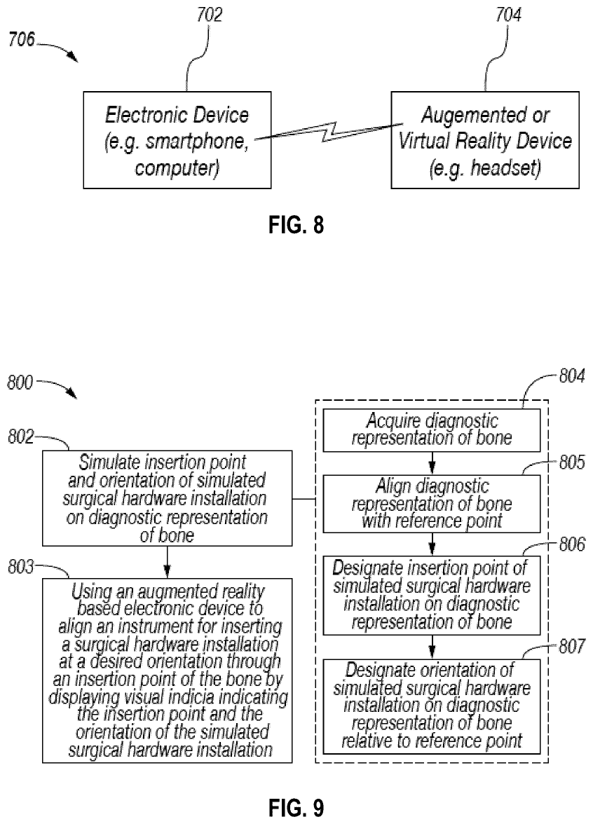

An augmented reality or virtual reality based system 706 for use in assisting of the determination of the proper insertion point and proper angle for a surgical tool to be used to install a pedicle screw is now described with reference to FIG. 8. The system 706 includes an electronic computing device 702, such as a smartphone, tablet, desktop based personal computer, or laptop based personal computer. A virtual reality based or augmented reality based device 704, such as a wearable headset, wearable goggles, three dimensional projector, or holoprojector, may be capable of wired or wireless communication with the electronic computing device 702.

Operation of the system 706 is now described with reference to the flowchart 800 shown in FIG. 9. Operation begins with the electronic computing device 702 simulating an insertion point and orientation of a surgical hardware installation on a diagnostic representation of the bone onto which it is to be installed (Block 802). This operation can proceed in any of the ways described above, although it should be understood that the virtual reality based or augmented reality based device 704 may be used as a display during this process. It should further be appreciated that the virtual reality or augmented reality based device 704 may have a camera associated therewith used to image the real world and provide it to the user when operating in an augmented reality mode (Block 803).

One way to proceed with this simulation begins with acquiring a diagnostic representation of the bone (Block 804). This may be performed using an image capturing device associated with the electronic computing device 702, such as a two dimensional or three dimensional camera, or this may be performed using a standalone image capturing device and then receiving the image data from that device at the electronic computing device 702. Still further, this may be performed using a medical imaging device, such as a CT scan or MRI scan, and then receiving that image data at the electronic computing device 702, which may serve as apparatus 300.

Thereafter, the diagnostic representation of the bone is aligned with a suitable reference point (Block 805). Then, an insertion point of for a simulated surgical hardware installation is designated on the diagnostic representation of bone (Block 806). Next, an orientation of the simulated surgical hardware installation on the diagnostic representation of bone relative to reference point is determined (Block 807). This orientation is determined in three dimensions, and can be referenced to suitable planes of the body as defined by typical medical terminology and known to those of skill in the art.

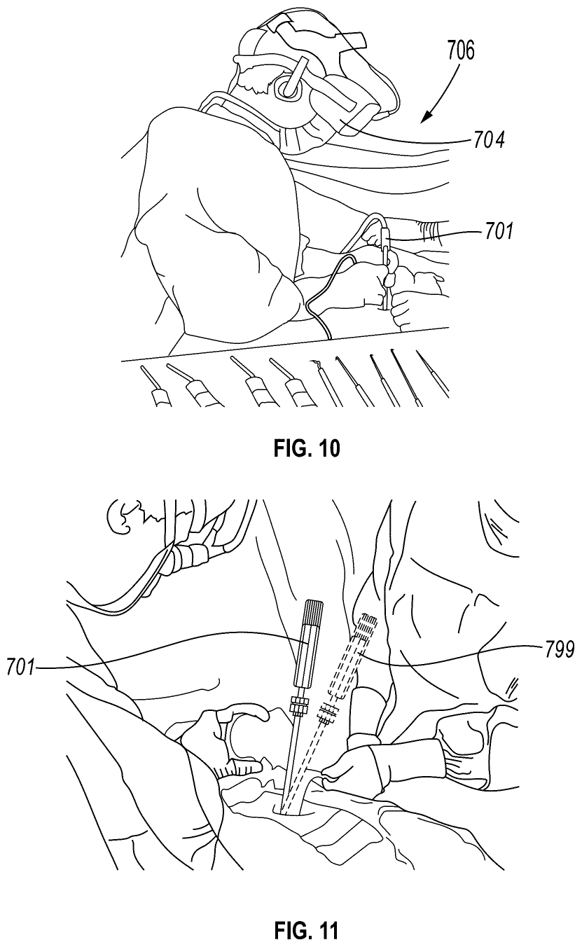

Then, the surgery itself may be performed. During surgery, virtual reality based or augmented reality based device 704 is worn by the operating physician or surgeon, as shown in FIG. 10. Here, the virtual reality or augmented reality based electronic device 704 is used to align an instrument or tool 701 for inserting a surgical hardware installation at a desired orientation through an insertion point of the bone by displaying visual indicia indicating the insertion point and the orientation of the simulated surgical hardware installation (Block 803). This visual indicia can be shown superimposed over the bone itself, such as shown in FIG. 11 by the virtual representation 799 of the tool 701. It should be appreciated that the visual indicia need not be a virtual representation 799 of the tool 701 as shown, and may instead be an arrow, a line, or any other suitable visual representation.

In some instances, cameras, position detectors, or other devices situated about the surgery site may be used to gather real time information about the actual position of the tool 701, so that feedback may be presented to the surgeon. For example, the visual indicia may change when the tool 701 is properly aligned, or may inform the surgeon that the tool 701 is not properly aligned. Likewise, additional visual indicia may be displayed when the tool 701 is properly aligned, or when the tool 701 is not properly aligned. Similarly, an audible response may be played by the virtual reality based or augmented reality based device 704 either when the tool 701 is properly aligned, or when the tool 701 is not properly aligned, or to guide the surgeon in moving the tool 701 into the proper position. In some cases, a position detector may be associated with or collocated with the tool 701, and the position detector such as an accelerometer may be used in determining whether the tool 701 is properly aligned, or when the tool 701 is not properly aligned.

In some instances, based on the above feedback, if the patient moved or the bone is moved, the visual indicia 799 is moved along with the bone by the virtual reality based or augmented reality based device 704 so that proper alignment is maintained during the surgery.

FIG. 12 illustrates a virtual representation presented by the system, such as the medical alignment device or electronic device of FIG. 8, showing a diagnostic image of a vertebra in an axial view with a simulated pedicle screw 210 shown that can be manipulated and moved to set a desired insertion point or location, and a desired alignment angle. Once set, an insertion location and alignment angle are stored, such as by a medical alignment device 300, for this two-dimensional view of the vertebra or object in this plane.

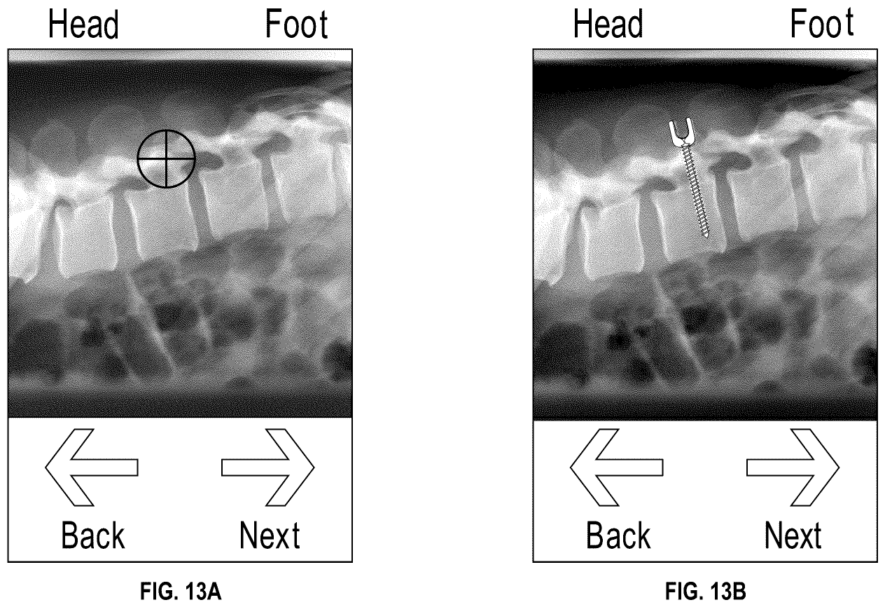

FIGS. 13A and 13B illustrate a virtual representation showing an orthogonal, lateral view of the vertebra and pedicle screw as shown and as set in the plane of FIG. 12, with the user able to establish or set the insertion location and alignment angle of the simulated pedicle screw in this plane so that the system, such as a medical alignment device, now has enough information as to the location of the pedicle screw in two orthogonal planes to determine a three-dimensional alignment angle for the installation of the pedicle screw (or drilling of a pilot hole for the pedicle screw) in this vertebra. FIG. 13A illustrates the cross-hair to set the desired insertion point, while being constrained with the positioning of the pedicle screw as defined in the view of FIG. 12, and, similarly, the angle of the pedicle screw may be set as desired as shown in FIG. 13B, while also being constrained with the positioning of the pedicle screw as set in the view of FIG. 12.

The medical alignment device 300 may calculate a desired three-dimensional alignment angle based on the inputs as just described in connection with FIGS. 12 and 13. The medical alignment device 300, knowing its own orientation, may notify a user, such as a surgeon, when a side, surface, or portion of the medical alignment device 300 is oriented according to the desired three-dimensional alignment angle. Thus, the apparatus 300, which may be referred to as a medical alignment device 300 in certain implementations, may be positioned relative to a tool (such as adjacent to or abutted with) to align the tool to the desired three-dimensional alignment angle. The tool may include, for example, a drill or gear shift probe to create a pilot hole for installing a pedicle screw. The tool, of course, could be any tool to be aligned at a desired three-dimensional angle, including a Jamshidi needle, mini-blade, or robotic device.

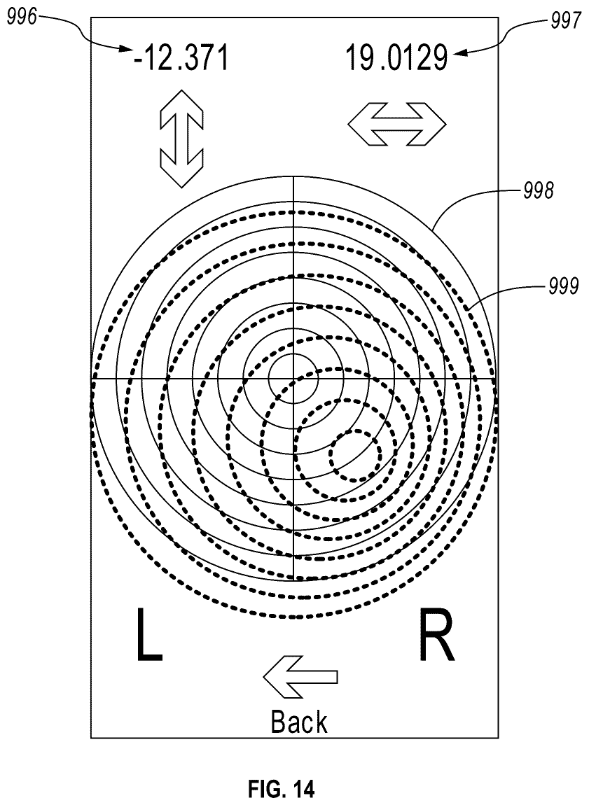

FIGS. 14-16 illustrate a series of two-sets of concentric circles illustrating one embodiment of a graphical indicator or notification showing how the current position of the apparatus 300 is oriented relative to the desired alignment angle. As the orientation of the apparatus 300 is moved or aligned more closely to the desired three-dimensional alignment angle, as illustrated when looking at FIGS. 14-16 consecutively, the concentric circles are moved closer to one another providing a graphical indication or feedback to assist a user or surgeon to align the apparatus 300, and hence an attached or adjacent tool, to the desired alignment angle. Once the apparatus 300 is oriented within a desired threshold close to the three-dimensional alignment angle, an auditory, visual, and/or tactile notification may be provided to alert the user.

Numerical indicators 996 and 997 may also be provided as shown in FIGS. 14-16, along with double arrows adjacent the numerical indicators to denote alignment in each such plane. The apparatus 300 may display numerical differences (or errors) in each of the two planes of the desired alignment angles. The numerical indicators 996 and 997 show how close and in what direction the orientation of the apparatus 300 is positioned relative to the desired alignment angles in each of the two planes or two-dimensions as previously set and stored in the apparatus 300.

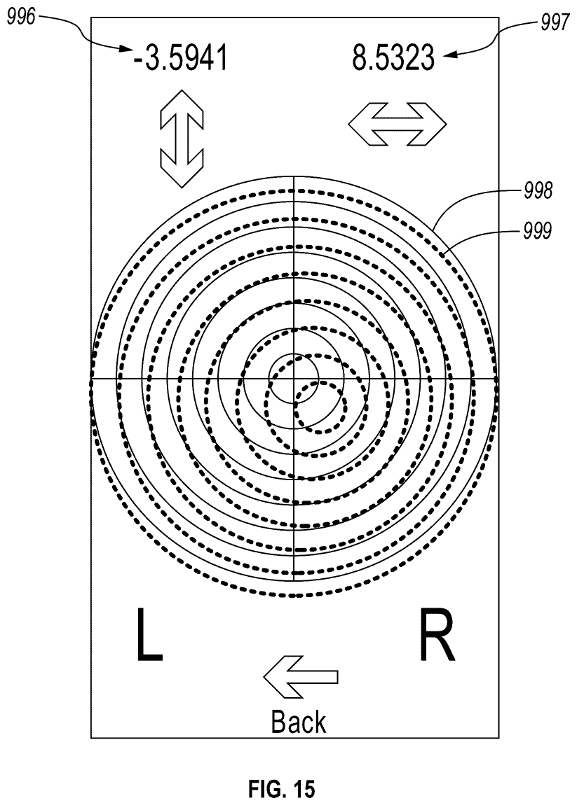

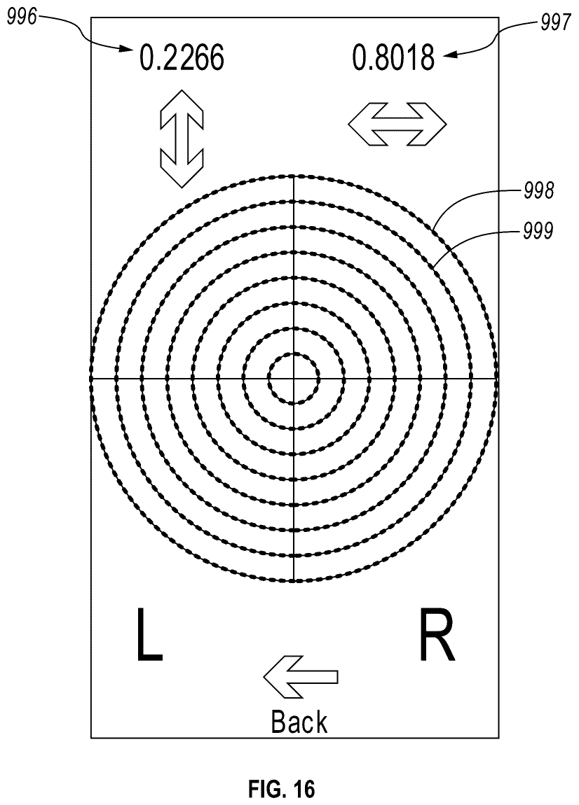

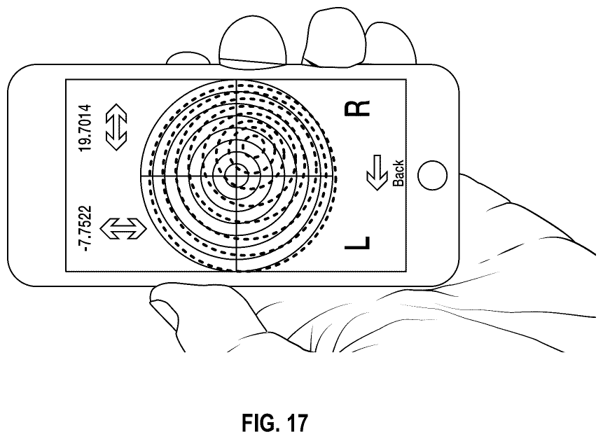

For example, FIG. 14 is a sample display of the apparatus 300 with two sets of concentric circles 998 and 999. In some implementations, the set of concentric circles 998 represents the desired three-dimensional alignment angle or orientation, such as the orientation of a pilot hole for a pedicle screw, while the set of concentric circles 999 represents the current three-dimensional orientation of the apparatus 300 showing the current orientation of the apparatus 300. As the apparatus 300 is oriented closer and closer to the desired three-dimensional alignment angle in FIGS. 15 and 16, the set of concentric circles 999 moves closer to the set of concentric circles 998 until the sets of circles are positioned over one another, or within a specified threshold, as illustrated in FIG. 16, to indicate that the apparatus 300 is aligned according to the desired three-dimensional alignment angle.

Similarly, the numerical indicators 996 and 997 in each of their respective planes are shown moving closer to zero, or within a specified threshold, as the apparatus 300 is moved closer and closer to the three-dimensional alignment angle when viewing FIGS. 14-16.

In some implementations, FIG. 15 is a sample display of the apparatus 300 in generating an indicator on the display 360 that indicates a degree of alignment between a tool aligned with a pedicle screw (or pilot hole or tool to install the pedicle screw) and the desired alignment angle, which may include an insertion sagittal angle, transverse angle, and/or coronal angle between an axis of the apparatus 300 and the sagittal plane, transverse plane, or coronal plane of the vertebra. As can be seen in FIG. 15, the indicator is in the form of a first set of concentric circles 998 and a second set of concentric circles 999. As the degree of alignment between the pedicle screw and the insertion sagittal angle, transverse angle, or coronal angle between an axis of the apparatus and the sagittal plane, transverse plane, or coronal plane of the vertebrae changes, the position of the first set of concentric circles 998 and position of the second set of concentric circles changes 999, or the position of one of the sets of the concentric circles 998 or 999 changes with respect to the other.

For example, as shown in FIG. 15, the set of concentric circles 999 is moved and positioned downward and to the right with respect to the set of concentric circles 998. This indicates that the proper alignment has not been found. By reorienting the apparatus 300, which it is noted would be directly or indirectly coupled to the pedicle screw or pilot hole location, in the appropriate direction, the set of concentric circles 999 moves closer to alignment with the set of concentric circles 998, as shown in FIG. 16. Once the proper alignment of the pedicle screw and the desired three-dimensional insertion angle between an axis of the apparatus and the vertebra has been reached, the sets of concentric circles 998 and 999 overlap one another, becoming one and the same, as shown in FIG. 16.

It can be noted that the color of the concentric circles 998 and 999 may be changed to further illustrate the degree of alignment between apparatus 300 and the desired alignment angle. For example, the misalignment indicated in FIG. 14 could be indicated by the set of concentric circles 999 being red, with the set of concentric circles 998 being blue; the better, but still not ideal, alignment indicated in FIG. 15 could be indicated by the set of concentric circles changing from red to yellow; and the ideal alignment indicated in FIG. 16 can be shown with both sets of concentric circles 998 and 999 being green.

It should be appreciated that although concentric circles have been shown, any concentric shapes can be used instead. In addition, concentric shapes need not be used, and any two individual shapes of the same size, or of a different size, may be used. Furthermore, it should be appreciated that in some instances one set of shapes may deform with respect to one another, in other instances both sets of shapes may remain at their original dimensions during operation.

In addition, in some instances, numerical indicators 996 and 997 may indicate the degree of alignment between the apparatus and a desired angle in a plane, a two-dimensional angle, such as the desired insertion sagittal angle, transverse angle, or coronal angle.

FIG. 17 illustrates the example of implementing the apparatus 300 as a smartphone or smart device application, with the sets of concentric circles and numerical indicators displayed and showing relative alignment of the apparatus 300 with a desired alignment angle, such as was shown in FIGS. 14-16. The apparatus 300 includes orientation circuitry/apparatus, such as a gyroscope, to know its three-dimensional orientation.

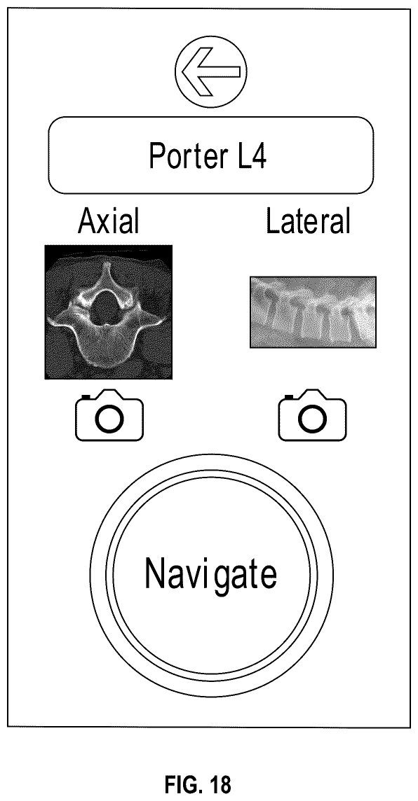

Shown in FIG. 18 is a user interface of the apparatus 300 of FIG. 3A in operation when selecting different diagnostic image views of a vertebra that are orthogonal to one another in preparation for establishing desired alignment angles so that the three-dimensional alignment angle may be determined to install a pedicle screw. Also, a patient may be identified, as well as the specific vertebra is identified. The diagnostic images may be provided to the apparatus 300 by digital transmission, or by using a camera of the apparatus 300 to capture these two images of the vertebra that are orthogonal to one another.

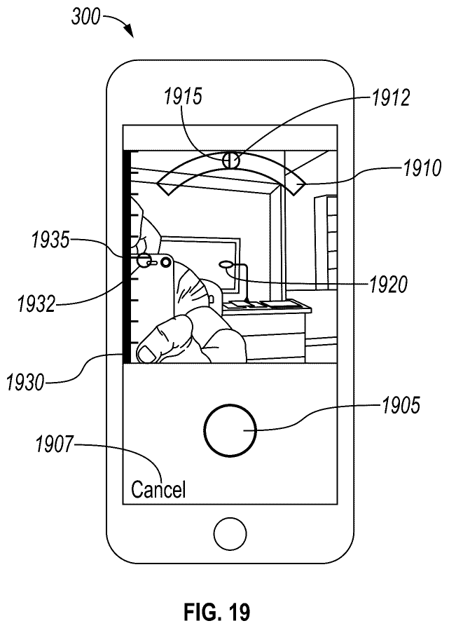

FIG. 19 illustrates a graphical user interface (GUI) of an orientation calibration system implemented using a smart device, such a smartphone, iPhone, iPod Touch, iPad, tablet computer, and the like. For example, the orientation calibration system may be implemented as part of the medical alignment device 300 (also referred to as apparatus 300 or orientation calibration system 300) to ensure that the medical alignment device is properly oriented or aligned when acquiring an image, such as a diagnostic image, appearing on an external display monitor. The diagnostic image may be acquired using a camera (not shown, and located on the other side) of the apparatus 300. The user interface may include a capture button 1905 (which may be thought of, or function as, a shutter button of a digital camera), a cancel button 1907, and an active viewfinder (the display capturing a live or current view using the camera of the device). For example, the diagnostic image to be captured may be displayed on a monitor as shown in the live view in FIG. 19 in the user interface. The display monitor shown in the live view is external to the apparatus 300 and may be referred to as an imaging source 1920. This may be a monitor to display any image, such as for example, a diagnostic medical image such as a CT or MRI scan. Inside the viewfinder or display of the apparatus 300, one or more graphical elements, such as dynamic graphical elements, may be provided to aid in displaying the present orientation of the apparatus 300, which may include a gyroscope or some other orientation sensor. In the illustrated example, dynamic graphical element includes a circle 1912 movable in a curved track 1910. The circle 1912 may change its color when the difference between the present orientation of the apparatus 300 and reference orientation is within a threshold value. The curved track 1910 may be indicated with a center position 1915, for which the user is intended to align the circle 1912. The tilt of the apparatus 300 to the left or right, In some implementations, should move the circle in each direction in the curved track 1910. This dynamic graphical element may be referred to as a left/right indicator, alignment, or orientation of the apparatus 300, and detects orientation, rotation, or alignment along, for example, a first axis, such as a “z” axis extending into and out of the page. This determines the position or orientation of the apparatus 300 along at least one axis.

The dynamic graphical element may further include a vertical indicator, such as a vertical gauge 1930 indicating a tilt of the medical alignment device 300 into or out of the page, In some implementations. The vertical gauge 1930 may include a center position 1935 and a circle 1932 movable along or adjacent the vertical gauge 1930. When the center (or some desired portion) of the circle 1932 reaches the center position 1935, the medical alignment device 300 becomes vertical and aligned with the gravitational direction (also referred to as orthogonal to the ground) or some other desired reference direction. This dynamic graphical element may be referred to as an up/down indicator, alignment, or orientation of the apparatus 300, and detects orientation, rotation, or alignment along, for example, a second axis, such as an “x” axis extending left to right on the page (or horizontal to the ground with the ground at the bottom of the page). This determines the position or orientation of the apparatus 300 along at least one axis.

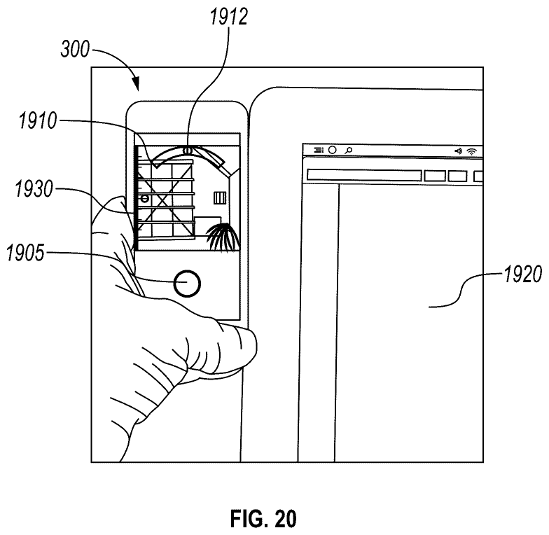

FIG. 20 illustrates an operation of using an orientation calibration system 300 to calibrate or align an imaging source 1920, which may be a computer monitor, external monitor, or any object where a target image is located. In certain applications, such as medical applications, having the imaging source 1920, such as a monitor displaying a diagnostic medical image that will be used in a medical alignment device, the need to ensure that the imaging source 1920 is properly oriented or aligned so that the image is not skewed when taken or captured by the medical alignment device. As shown, the imaging source 1920 is calibrated or adjusted to a desired orientation. This may be achieved by utilizing the orientation sensor of the apparatus 300 with a built in orientation sensor and the dynamic graphical elements described above. This apparatus 300 may be placed adjacent (or abutted against) certain sides, edges, or locations of the imaging source 1920 to ensure that the imaging source may be adjusted and aligned as desired. In one example, the apparatus 300 is first aligned to a known edge or side of the imaging source 1920 such that, in one example, they are coplanar and having at least one edge aligned, adjacent, and/or abutting one another as shown in FIG. 20.

The orientation sensor in the apparatus 300 may be active and shows the present orientation relative to a known reference orientation, such as a calibrated orientation or the ground. In some implementations, the user may use the present orientation as the calibrated orientation or redefine the calibrated orientation, in certain implementations. The user may adjust the orientation of both the apparatus 300 and the imaging source 1920 to desired position or orientation. In some implementations, the user desires that the display screen of the imaging source 1920 is perpendicular to the ground and all sides of the imaging source 1920 are orthogonal to one another and to the ground. This may be achieved, In some implementations by (i) aligning the edge of the apparatus 300 adjacent a straight, left edge of the imaging source 1920, as shown, and adjusting the imaging source 1920 using the circle 1912 and the curved track 1910 until the left edge of the imaging source 1920 is vertical and orthogonal to the ground, and (ii) aligning the back of the apparatus 300 adjacent the flat face (or surface) of the display screen of the imaging source 1920, as shown, and adjusting the orientation of the imaging source 1920 using the circle 1932 and the vertical gauge 1930 until the face of the display screen of the imaging source 1920 is vertical and orthogonal to the ground. As such, two axes of rotation are aligned, and the imaging source 1920 may display a target image, such as a medical diagnostic image, that is positioned orthogonal to the ground. The apparatus 300 may then be used to capture or take a picture of that image displayed on the imaging source 1920 while the apparatus 300 itself, including the camera of the apparatus 300, is positioned orthogonally to the ground as well. This enhances the accurate capture of such target image, and reduces skew or errors, which are often not readily visible, that are introduced by capturing images at angles that are not properly aligned.

In some implementations, a default orientation may be used, such as one of the sagittal plane, the transverse plane, the coronal plane, or planes orthogonal to the ground. The user may report the calibrated orientation by noting the relative positions between the circle 1912 and the curved track 1910, in the circle 1932 and the vertical gauge 1930. If the apparatus 300 captures the target image from the imaging source 1920 at the same default orientation, an accurate target image may be obtained.

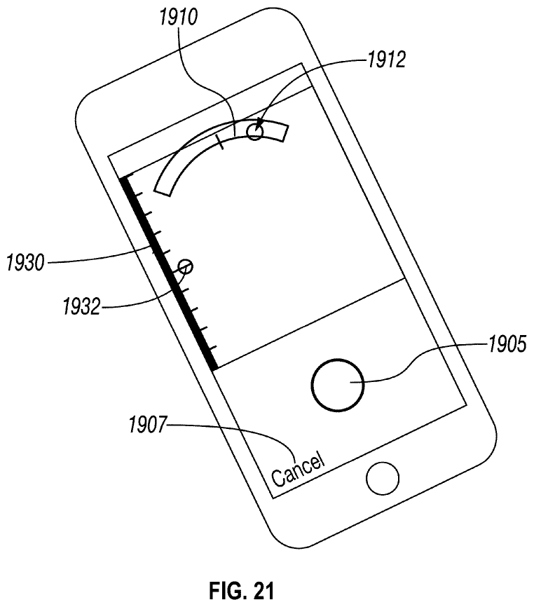

FIG. 21 illustrates a GUI, such as that shown in FIG. 19, of an orientation calibration system when the medical alignment device 300 (which may also be referred to as the apparatus 300 or the orientation calibration system 300) is out of the proper or desired orientation. Because the apparatus 300 is shown tilted to the “left” on the page while positioned on a flat surface parallel to the ground, for example, the circle 1912 is far away from the center position 1915 on the track 1910 indicating the “left” orientation of the apparatus 300, while the circle 1932 is positioned in the middle or adjacent the center position of the vertical gauge 1930 indicating that the back surface of the apparatus 300 is orthogonal to the ground. If the apparatus was tilted to the “right”, the circle 1912 would be on the other side from the center position 1915 on the track 1910 indicating the “right” orientation of the apparatus 300 in such a case.

Once the imaging source 1920 is properly oriented, a user may use the apparatus 300 to capture a target image displayed on the imaging source 1920. In doing so, it can be important that the apparatus 300, which includes a camera, is properly aligned when capturing such target image. Thus, the same alignment tools of the apparatus 300 used to align and properly orient the imaging source 1920, including the dynamic graphical elements such as the circle 1912 and the curved track 1910 as well as the circle 1932 and the vertical gauge 1930, may be used to ensure that the apparatus 300 itself is properly oriented before the target image is captured by the apparatus 300. It should be understood that the present disclosure is not limited to the specific dynamic graphical elements illustrated herein, and that any number of other dynamic graphical elements may be used to ensure a desired orientation or alignment of the apparatus 300. For example, the curved track 1910 may be a straight track.

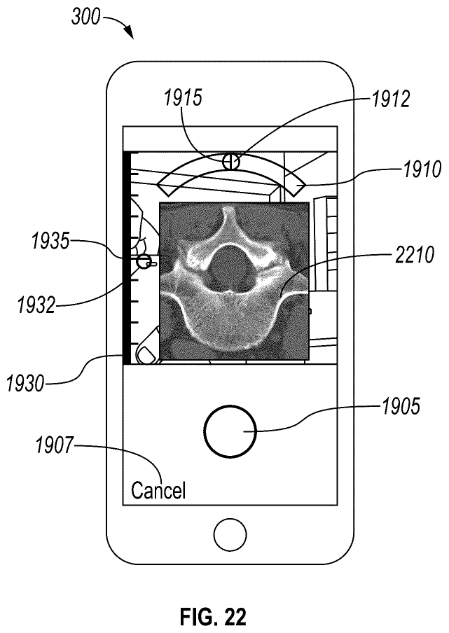

FIG. 22 illustrates an operation of using the orientation calibration system 300 to capture a target image, which may also be referred to as a reference image 2210, from an imaging source, such as a display or monitor with a diagnostic image being displayed. For example, when the apparatus 300 is properly oriented, such as when the circle 1912 reaches a predetermined range or threshold near or adjacent the center position 1915, and when the circle 1932 reaches a predetermined range or threshold of the center position 1935, the reference image 2210 may be captured by the camera of the apparatus 300. In some implementations, the processor of the medical alignment device 300 can automatically capture the reference image 2210 when alignment is achieved. In some other implementations, in response to the alignment, a user can capture the reference image 2210 by pressing the capture button 1905. If a capture reference image 2210 is not satisfactory, a user may cancel to capture reference image 2210 by operation of the cancel button 1907.

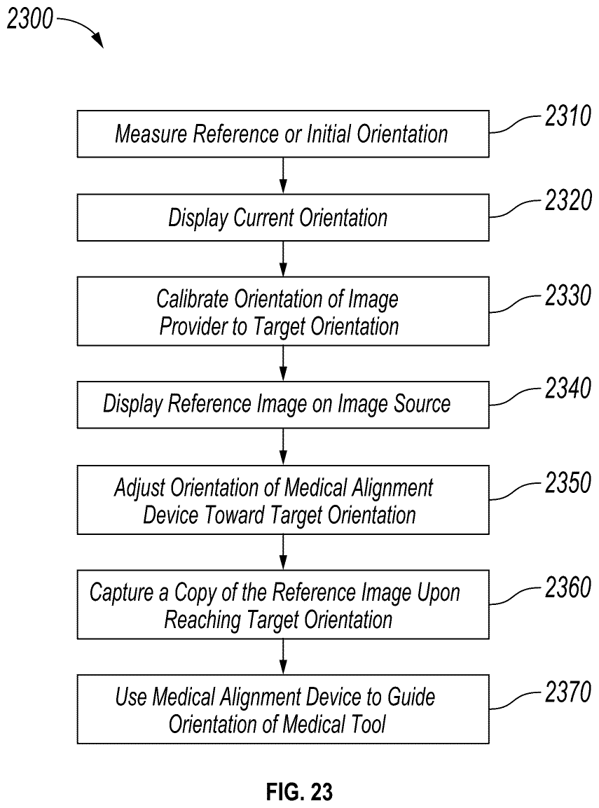

FIG. 23 is a flowchart 2300 showing an example of an orientation calibration process that may include one or more of a method for orienting a system for capture of a target image (or reference image), and a method for using an orientation calibration system to align a display monitor in an orthogonal position relative to the ground.

At 2310, the reference or initial orientation is measured. For example, the reference orientation may be an initial orientation recorded by the orientation sensor of the medical alignment device 300. Some implementations, the reference orientation may be a specific orientation defined by the user relative to a known reference frame. Subsequent measurement of the orientation change by the orientation sensor may be made with reference to the measured reference orientation. In some implementations, the reference orientation is already set and does not have to be set each time, and this may include a first axis orthogonal to the ground (a gravitational vector axis), with two additional axis each orthogonal to each other and each orthogonal to the first axis. This may be visualized as an x, y, z cartesian coordinate system in three-dimensional space.

At 2320, the current orientation of the apparatus 300 is displayed on a display screen of device, which may be an orientation calibration system or a medical alignment device, which we will use in describing the flowchart 2300. In some implementations, the current orientation may be displayed when other visual devices, wirelessly or by cable, are in communication with the medical alignment device. The current orientation may be represented by a dynamic graphical representation, such as a circle moving along a track or gauge or numerically. The current orientation of the medical alignment device may be shown, In some implementations, as two or three axis of rotation, and this information is provided by an orientation sensor using a gyroscope in the medical alignment device 300.

At 2330, the user calibrates the orientation of the imaging source, which may be a computer monitor, to a target orientation. For example, the target orientation may be the sagittal plane, the transverse plane, and the coronal plane, or orthogonal to the ground along a side edge, and parallel to the ground along a top or bottom edge.

At 2340, a reference image or target image is displayed by the imaging source, such as a display monitor. For example, an imaging source may be connected to a CT scanner that provides images of a patient. In some other implementations, the imaging source may be connected to a database storing images of the patient.

At 2350, orientation of the medical alignment device 300 is adjusted to the target orientation so that when the target image is captured by the camera of the apparatus 300, the image will not be distorted or skewed. For example, a user may hold the medical alignment device 300 and view the dynamic graphical representations of its current orientation on its display, such as by tracking the circles along a curved track or the vertical gauge as shown in FIGS. 19-22, until a camera of the medical alignment device 300 is properly aligned in front of the target image to properly capture such image being displayed by the imaging source.

At 2360 when a target orientation is reached, a copy of the reference or target image may be captured by the medical alignment device. For example, the processor of the medical alignment device 300 may capture the reference image automatically when the target orientation is reached. In other instances, a user may provide a command to capture the reference image in response to reaching the target orientation. The command may be by touch, may be by voice, and may include other sources of inputs.

At 2370, the now calibrated medical alignment device 300, in certain implementations, may be ready to guide orientation of the medical tool, for example, as discussed in FIG. 7.

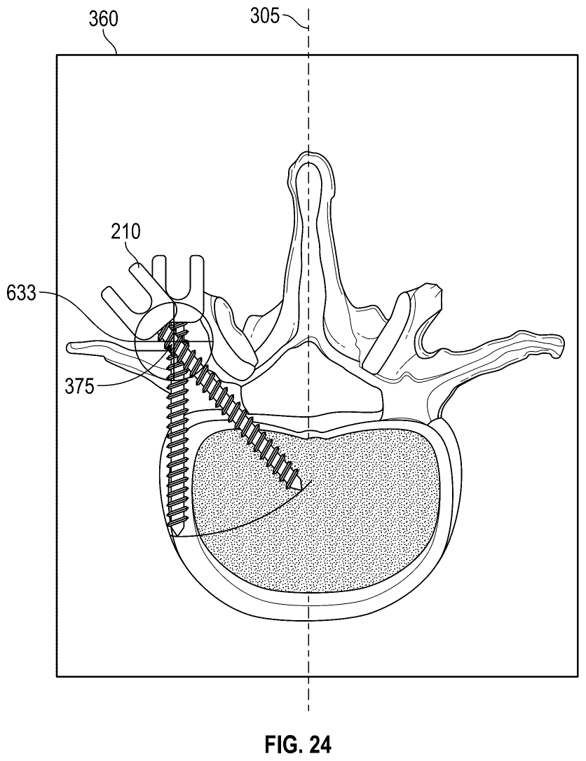

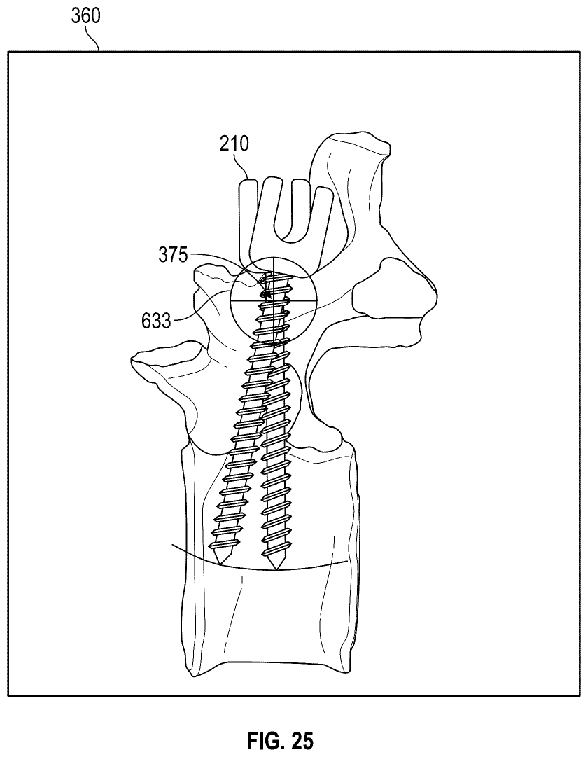

Referring now to FIGS. 24 and 25, a schematic diagram of a transverse view and a lateral view, respectively, of a vertebra defining an alignment or insertion angle for a pilot hole in the vertebra (e.g., any bone) in this plane for insertion or installation of a pedicle screw is provided. For instance, if the vertebrae in FIG. 24 was rotated 90 degrees about axis 305, the same vertebrae with the same insertion angle for the same pedicle screw can be viewed in FIG. 25. These views or diagnostic images of the vertebra may be electronically transmitted to the medical alignment device 300, or the views or images may be captured from a monitor or display of the diagnostic images using the image acquisition unit 320 of the medical alignment device 300 (sometimes referred to as apparatus 300). The display 360 shows the field of view of the view captured by the image acquisition unit 320, assuming that was how the images were acquired, and allows a user to align the axis 305 of the apparatus 300 with the desired plane. For instance, the user may view the image (e.g., a still image previously captured and communicated to the apparatus 300) with respect to the axis 305 such that the provided image is aligned with the axis 305. The views as displayed in FIGS. 24-25 are each fixed images provided along different planes. In other words, FIGS. 24-25 are not meant to illustrate a user rotating the views.

Simulating the insertion point 375 (e.g., the initial position, the insertion location, etc.) and the orientation of the simulated surgical hardware installation on the diagnostic representation of the bone includes acquiring the diagnostic representation of the bone, providing the diagnostic representation of the bone with a reference point (e.g., the crosshairs 633), and designating the insertion point of the simulated surgical hardware installation on the diagnostic representation of the bone with the reference point.

As explained above, definitions of the insertion angle of the pilot hole 220 and the initial position 375 of the pilot hole 220 (e.g. see FIG. 3B) are provided or specified by a user. For instance, the insertion location or initial position 375 of the pilot hole 220 for the installation of a pedicle screw are established by locating (or simulating) graphically the insertion location on the displayed diagnostic image, and the applicable alignment angle for the displayed plane may be defined by moving or locating (or simulating) the desired position of the alignment angle of the pilot hole/pedicle screw. Further, the user next selects the optimal pedicle screw position by selecting the navigation button 644 (e.g., FIG. 6C) to move the simulated pedicle screw to a desired location by moving the crosshairs 633 (e.g., a movable marker; see FIG. 6C) to the cortical entry point of the screw, for example, by tapping the entry point button 632 to confirm, and then tapping the trajectory button 634 and rotate the screw to its desired position 635. The crosshairs 633 specify the insertion position 375.

Simulating the orientation of the simulated surgical hardware installation further includes rotating the simulated surgical hardware installation about the insertion point on the diagnostic representation of the bone, and designating the orientation of the simulated surgical hardware installation on the diagnostic representation of the bone relative to the insertion point. Once inserted, the surgical hardware device (e.g., the pedicle screw 210) is shown in the simulated position in the vertebra through the insertion point 375, the pedicle screw 210 may be moved or rotated in this view about the insertion point 375. Rotating the simulated surgical pedicle screw 210 about the insertion point 375 includes rotating the pedicle screw 210 from left and right from the transverse view, or up and down (i.e., left and right from the lateral view). For instance, once the angle relative to the transverse plane is set as in FIG. 24, the user may view the image as in FIG. 25 (e.g., a view of the same vertebra in FIG. 24 rotated 90 degrees to the lateral view) of the patient bone by selecting a Next button to determine where the pedicle screw would reside in a third dimension. In other words, the first plane is a first fixed image to adjust the simulated pedicle screw in a first angle at which point the pedicle screw can be rotated in that direction, and the second plane is a second fixed image to adjust the simulated pedicle screw in a second angle at which point the pedicle screw can be rotated in that direction. Thus, the pedicle screw can be adjusted via a three-dimensional orientation as rotation is simulated about the entry point.

It should be understood that there is a single, rotating pedicle screw illustrated in each of FIGS. 24 and 25. For instance, separate images are shown in FIG. 24 and FIG. 25 illustrating the same pedicle screw rotated in different angles relative to the insertion point at different planes. The retake button 624 allows the user to go back to retake an image to ensure the alignment is proper.

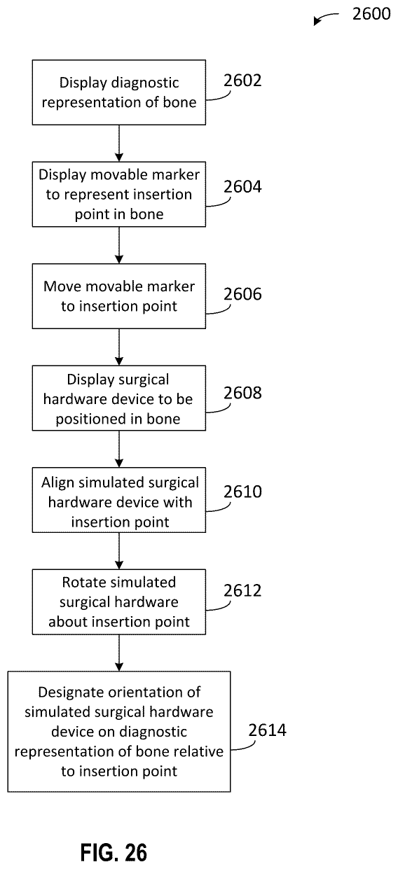

Referring now to FIG. 26, a method 2600 for simulating a three-dimensional position of a surgical hardware device in a bone using a diagnostic representation of the bone is shown. At block 2602, the diagnostic representation of the bone (e.g., vertebrae) is displayed. The diagnostic representation may be a pictorial view of the bone, an x-ray of the bone, a radiograph of the bone, a computed tomography scan of the bone, a magnetic resonance image of the bone, or any known or available diagnostic image. At blocks 2604-2606, a movable marker, such as crosshairs 633 described above, to represent an insertion point in the bone along with the diagnostic representation of the bone is displayed and moved to the insertion point in the bone as represented by the diagnostic representation of the bone. At block 2608, the surgical hardware device to be positioned in the bone along with the diagnostic representation of the bone is displayed. At block 2610, the simulated surgical hardware device is displayed and aligned with the insertion point. At block 2612, the simulated surgical hardware device is rotated about the insertion point to a desired location within the vertebra. Rotating the simulated surgical hardware device about the insertion point includes rotating the surgical hardware device from left and right in a transverse view and left and right in a lateral view (see e.g., FIGS. 24-25). Thus, at block 2614, the orientation of the simulated surgical hardware device on the diagnostic representation of the bone is designated relative to an insertion point.

In various implementations, the method 2600 may implement an augmented reality based electronic device to assist with the process described above (e.g., aligning the simulated surgical hardware device at a desired orientation through the insertion point of the bone by displaying visual indicia indicating the insertion point and the orientation of the simulated surgical hardware device). For instance, the visual indicia (e.g., a line representing the insertion point and the desired orientation angle) indicating the insertion point and the orientation of the simulated surgical hardware device are displayed superimposed on the bone. The desired orientation is a desired angle between the electronic device and a plane of the bone represented in the diagnostic representation of the bone.

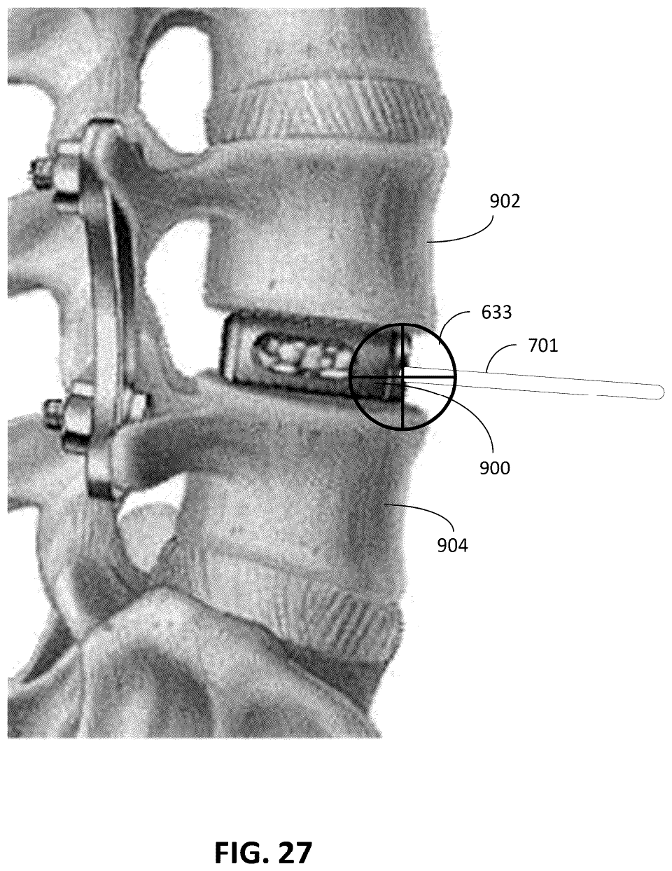

Referring now to FIG. 27, an alternative embodiment of a medical device installed in a body is illustrated. Particularly, an image of a medical device 900, such as an interbody cage or artificial disc, is depicted. The interbody cage can be seen disposed between two vertebral bodies 902, 904 of a spine. Although exemplary implementations of the implementations have been described for installing a medical device, such as a pedicle screw, into a bone, the device may be used to install, position, or align any surgical/medical hardware and assemblies into or adjacent to any part of a body. The device may be used on both human and non-human bodies (e.g., other mammals, reptiles, etc.). For instance, the device may be used to simulate positioning a medical device (e.g., an interbody cage) within a body (e.g., implanted between vertebral bodies of the spine). This operation is performed when a disc is removed and an interbody cage, or other device, is needed to be placed where the disc once was. The process of clearing out the disc space can be approached from the front, back, or side of the patient. The disc space is cleaned out with a variety of instruments. Specifically, the interbody cage can be coupled to an instrument 701 (e.g., an inserter; see also FIGS. 10 and 11) to facilitate installation of the interbody cage into the disc space. For instance, the interbody cage may include a threaded opening configured to threadedly couple to an end of the instrument 701. As such, when the instrument 701 is aligned as desired, as described further herein, the interbody cage will be properly aligned as desired.

Similarly to FIGS. 24 and 25, an axial and a lateral view may be provided for the simulated alignment of the medical device (e.g., interbody cage) on a diagnostic image of at least a portion a patient's body. These views or diagnostic images of the vertebra (or other portions of a body in which a medical device may be installed or implanted) may be electronically transmitted to the medical alignment device 300, or the views or images may be captured from a monitor or display of the diagnostic images using the image acquisition unit 320 of the medical alignment device 300 (sometimes referred to as apparatus 300). The display 360 shows the field of view of the view captured by the image acquisition unit 320, assuming that was how the images were acquired, and allows a user to align the axis 305 of the apparatus 300 with the desired plane (see FIG. 3A). For instance, the user may view the image (e.g., a still image previously captured and communicated to the apparatus 300) with respect to the axis 305 such that the provided image is aligned with the axis 305.

Simulating the orientation and installation of the simulated medical device, also referred to as the surgical hardware, on a diagnostic representation of at least a portion of a body (e.g., a spine) includes acquiring the diagnostic representation, providing the diagnostic representation of the at least a portion of the body with a reference point (e.g., the crosshairs 633 representing a desired location within the body), and designating the insertion point of the simulated surgical hardware on the diagnostic representation with the reference point. In some implementations, the insertion point need not be designated.

As explained above, definitions of the insertion angle of the pilot hole 220 and the initial position 375 (insertion or entry location) of the pilot hole 220 (e.g. see FIG. 3B) are provided or specified by a user. For instance, the insertion location or initial position 375 of the pilot hole 220 for the installation of a medical device are established by locating (or simulating) graphically the insertion location on the displayed diagnostic image, and the applicable alignment angle for the displayed plane may be defined by moving or locating (or simulating) the desired position of the alignment angle of the pilot hole for the instrument 701. For instance, the alignment angle corresponds with the inserter 701 coupled to the interbody cage to simulate the installation of the interbody cage. Further, the user can select the optimal interbody cage position by selecting the navigation button 644 (e.g., FIG. 6C) to move the simulated interbody cage to a desired location by moving the crosshairs 633 (e.g., a movable marker; see FIG. 6C) by tapping the entry point button 632 to confirm, and then tapping the trajectory button 634 and rotate the inserter 701 to its desired position 635. The crosshairs 633 specify the installation position 375. The crosshairs 633 may indicate the target location of the inserter 701, where the inserter 701 is coupled to the medical device 900, or the crosshairs 633 may be used to indicate the desired location of the medical device 900 separately.

Once the angle relative to the axial view is set (similarly to FIG. 24, the user may view the image as in FIG. 27 (e.g., a view of the same portion of the body rotated 90 degrees to the lateral view) of the body by selecting a Next button to determine where the medical device would reside in a third dimension. In other words, the first plane is a first fixed image to adjust the simulated interbody cage in a first angle at which point the interbody cage can be positioned in that direction, and the second plane is a second fixed image to adjust the simulated interbody cage in a second angle at which point the interbody cage can be rotated in that direction. Thus, the interbody cage can be adjusted via a three-dimensional orientation as rotation is simulated about the entry point with the instrument, or inserter. The rotation of the inserter 701 coupled to the interbody cage can allow adjustment of the interbody cage until one or more surfaces of the interbody cage, or one or more edges of the interbody cage, align with one or more surfaces of one or more vertebrae as desired. However, as previously stated, installing an interbody cage between two vertebral bodies is an exemplary embodiment, whereas the apparatus 300 described herein may be used to install any medical hardware at any desired location within a body.

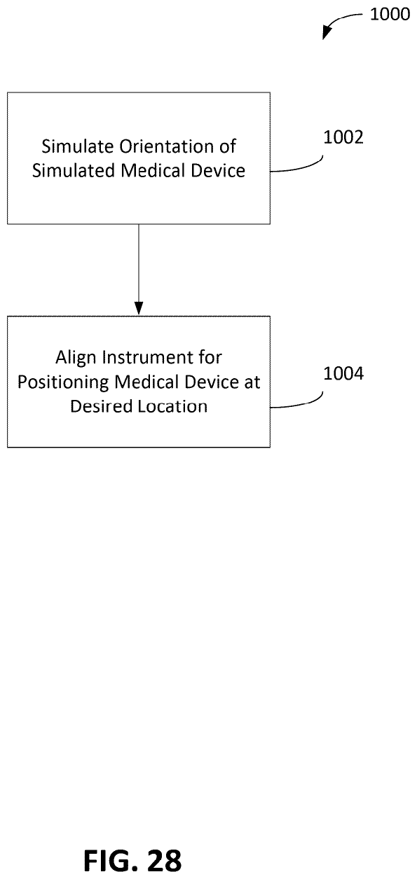

Referring now to FIG. 28, a method 1000 for determining orientation of an instrument for positioning a medical device in a body is illustrated. At 1002, a user can simulate an orientation of a simulated medical device 900 on a diagnostic representation of at least a portion of the desired location (e.g., using the crosshairs 633) in the body. In various implementations, this may include simulating a positioning of the medical device between vertebral bodies. However, the at least a portion of the body may be one or more portions of the body from a spine, a joint, a rib cage, a cranium, an artery, a lung, or other portion of the body to receive an implant. For instance, the medical device may be any medical hardware that includes an interbody cage, a pedicle screw, a steel rod, a stent, a bone graft, or other implant. At 1004, the user can align the instrument 701 for positioning the medical device 900 at the desired location in the body, according to the simulation of the simulated medical device 900, through an insertion point of the body by indicating when an orientation is within a threshold of the simulated orientation. In various implementations, the method 1000 may further include capturing an image of the representation of the vertebra, generating an angle-indicative line on a display of the electronic device, wherein the angle-indicative line adjusts in response to rotation and orientation of the simulated medical device, and generating a notification when the instrument is at the correct angle for positioning the medical device. An augmented reality based electronic device may be used to assist with aligning the simulated medical device at a desired orientation through the insertion point of the body by displaying visual indicia indicating the insertion point and the orientation of the simulated surgical hardware device. For instance, the visual indicia indicating the insertion point and the orientation of the simulated medical device are displayed superimposed on the diagnostic representation of the at least a portion of the body.

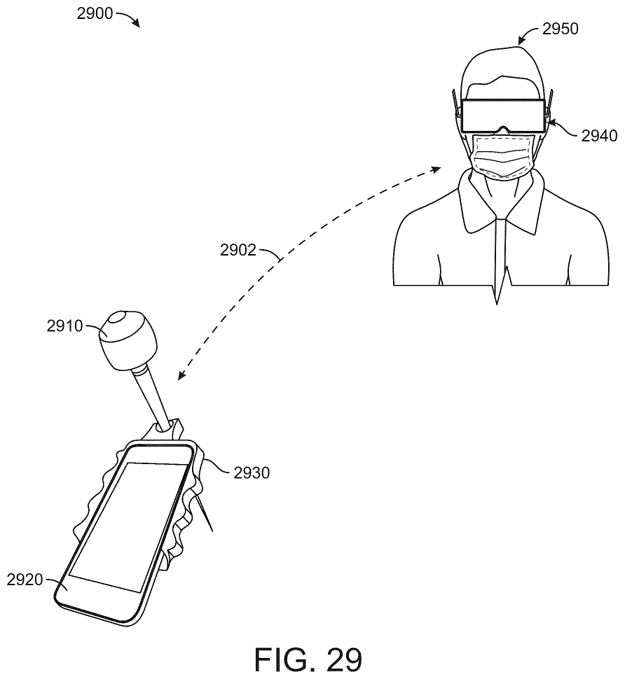

Referring now generally to FIGS. 29-32, illustrations of various configurations for orienting a surgical tool at a desired three-dimensional insertion angle at a desired location within an environment, according to example implementations. Generally, the configuration can include at least one of a smart headset 2940, an electronic device 2920, an electronic device 2960, and a surgical tool 2910. Each electronic device (e.g., 2920 and 2960) can be communicably coupled with other electronic devices and smart headset 2940 over a network (e.g., 2902, 2904, 2905, 2906, and 2908). The networks may include one or more of a cellular network, the Internet, Wi-Fi, Wi-Max, Bluetooth, a proprietary provider network, a proprietary retail or service provider network, and/or any other kind of wireless or wired network. Additionally, each electronic device (e.g., 2920 and 2960) can be coupled to a mounting device (e.g., mounting device 2930 for mounting electronic device 2920 or mounting device 2970 for mounting electronic device 2960) that fixedly couples the electronic device to the surgical tool 2910 or 2980. In some implementations, the surgical tool 2910 is a pedicle probe. However, in other implementations, the surgical tool 2910 may be a Jamshidi needle, a pedicle screw, a syringe, an interbody cage, a stent, a bone graft, a medical implant, or other medical appliance. Likewise, surgical tool 2980 may be a pedicle probe, gear shift probe, a Jamshidi needle, a syringe, a pedicle screw, an interbody cage, a stent, a bone graft, a medical implant (screw, pin, rod, etc.), or other medical appliance. These various tools, implants, appliances, and devices may be used in any embodiment described herein, including in aligning the tools, implants, appliances, and devices in a desired orientation at a desired location within the surgical environment. In some implementations, the surgical tool 2980 (or 2910) can include one or more geolocators that can communicate with the electronic devices described herein (e.g., 2920, 2940, 2960). For example, the geolocator of the surgical tool 2980 can establish a communication session with and transmit geolocation data (e.g., continually, continuously, or periodically) to the electronic devices.

In some implementations, the surgical tool 2910 (or 2980) is used in a percutaneous surgical operation such as a spinal fusion. In such implementations, the surgical tool 2910 (e.g., a Jamshidi needle) is inserted into the patient through the skin and placed at a desired location on a surface of a bone or other surgical location. The operation is performed without retracting tissue of the patient to create a surgical corridor. Rather, the operation is performed in a minimally invasive way so as to minimize the incision required to perform the operation.

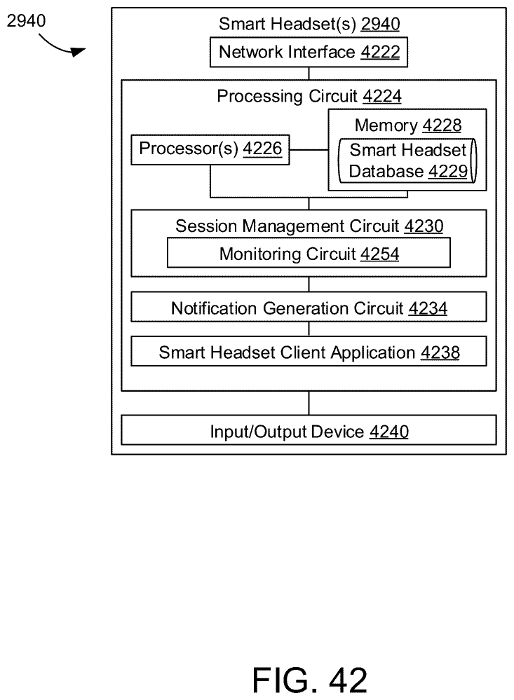

Each system or device (e.g., 2920, 2940, 2960) in the environment (e.g., 2900, 3000, 3100, 3200) may include one or more processors, memories, network interfaces (sometimes referred to herein as a “network circuit”) and user interfaces. The memory may store programming logic that, when executed by the processor, controls the operation of the corresponding computing system or device. The memory may also store data in databases. For example, memory 4228 of FIG. 42 may store programming logic that when executed by processor(s) 4226, which may be one or more processors, within processing circuit 4224, causes smart headset database 4229 to update information for orienting a surgical tool at a desired three-dimensional insertion angle at a desired location (e.g., for use in installing a medical device using and displaying at least one graphical element). The network interfaces (e.g., network interface 4222 of smart headset 2940) may allow the computing systems and devices to communicate wirelessly or otherwise. The various components of devices in the environments may be implemented via hardware (e.g., circuitry), software (e.g., executable code), or any combination thereof. Devices and components in FIGS. 29-32 can be added, deleted, integrated, separated, and/or rearranged in various implementations of the disclosure. In some implementations, one or more of the processing circuits described herein can be separate from the headset in that they may reside in an external computing device that communicates with the headset wirelessly or through a wired connection. For example, the processing circuits could be part of a dedicated server or a portable computing device like a smartphone (e.g., user device, such as 2920 and 2960), which handles computations and relays the results to the headset for display. In another example, the processing circuits could be housed in a wearable device such as a belt-mounted module or system that connects to the headset via a wired (or wireless) connection, offloading the processing tasks while maintaining a low-latency communication link with the headset. In some implementations, the one or more processors are enclosed within the smart headset (e.g., a unit or component enclosed within a structure)

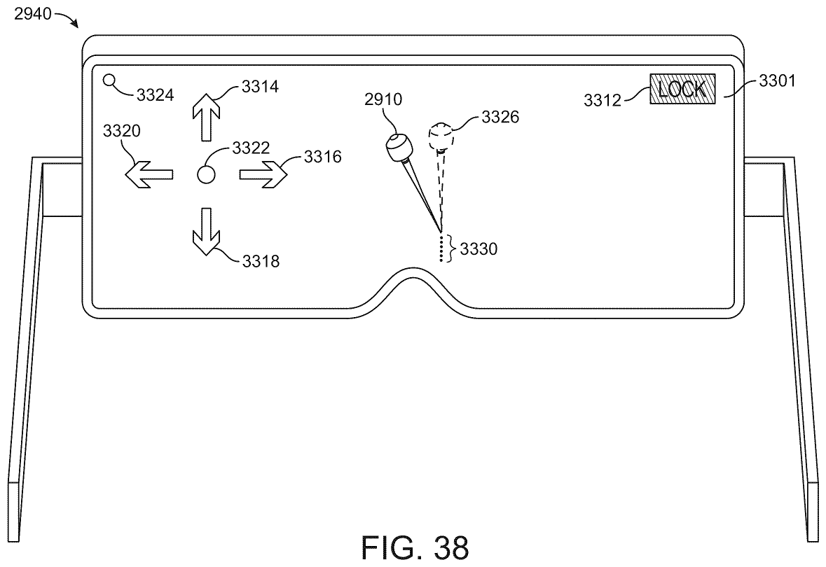

Still referring generally to FIGS. 29-32, the electronic devices (e.g., 2920 and 2960), in certain implementations, can be configured to collect environmental data within the environment. For example, the electronic device 2920 can collect environmental data indicating the position of the surgical tool 2910 (or 2980) within the environment. In some implementations, the environmental data can be continually (e.g., regularly or frequently) and/or continuously (e.g., without interruption) collected in real-time (or near real-time) for determining orientation (e.g., orientation data) of the surgical tool 2910 as it moves within the environment. Additionally, the continually (or intermittently, or continuously) collected orientation data can be transmitted (e.g., via a network) to the smart headset 2940 such that a graphical element superimposed within the environment (e.g., virtual surgical tool such as virtual surgical tool 3326 of FIG. 38) can be automatically updated in real-time (or near real-time). In various implementations, environmental data can include, but is not limited to, orientation data of the surgical tool (e.g., vectors, axes, planes, positions, etc.), orientation data of a portion of a body (e.g., transverse plane, coronal plane, sagittal plane, associated with anatomy of the portion of the body), room/area information, types of electronic devices, user information, etc. In various implementations.

Still referring generally to FIGS. 29-32, the smart headset 2940 can be configured to be initiated and perform various actions (e.g., receiving data, capturing data, transmitting data, generating graphical elements, displaying graphical elements, updating displayed graphical elements, determining orientations, etc.). In some implementations, initiate a session for performing a procedure (e.g., on a portion of a body, on anatomy of an animal), where the session can be between one or more electronic devices (e.g., 2920 and 2960) and the smart headset 2940. Initiating a session can include receiving a trigger event. For example, when a user 2950 wearing the smart headset 2940 is within a certain proximity or wireless communication range of the one or more electronic devices (e.g., 2920 and 2960). In response to entering the wireless communication range, the smart headset 2940 may be configured to automatically request from user 2950, via smart headset 2940, to provide a confirmation (e.g., audible, eye movement, selection, or any other type of feedback, etc.). A session trigger event may also include receiving an input via input/output device 4240 of smart headset 2940 of FIG. 42, such as receiving a user interaction via haptic feedback or other input via smart headset 2940. In other implementations, a session trigger event may include a user 2950 logging into a smart headset client application 4238 on smart headset 2940 or electronic device 2920. In additional implementations, a session trigger event may occur at a specific time, such as in response to the session management circuit 4230 determining there is a scheduled procedure at a specific time. In some implementations, the smart headset 2940 may be configured to operate in a low power mode or “sleep mode” until a session trigger event is received.

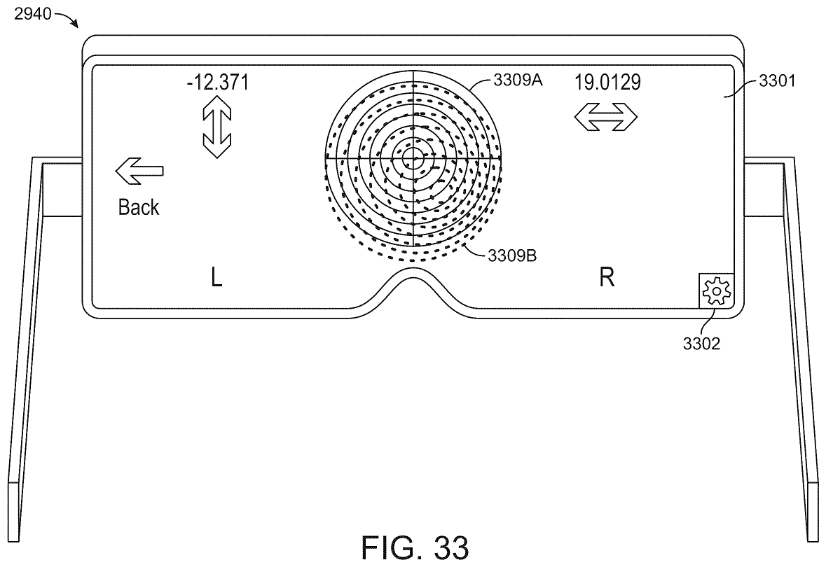

After a session trigger event is received, the smart headset 2940 can be initiated with the environment (e.g., 2900, 3000, 3100, 3200) such that smart headset 2940 can be calibrated to the environment so that the smart headset 2940 determines its position relative to the environment when the smart headset 2940 moves in the environment. The smart headset 2940 can determines (or calibrate) its position relative to the environment based on collecting and receiving various environmental data and sensor data via input/output device 4240 and/or accessing smart headset database 4229. For example, a camera of the smart headset 2940 can collect images and videos of the environment to determine various vectors and planes within the environment. In another example, the smart headset 2940 may access the smart headset database 4229 to determine the room/area configuration of the environment or medical imaging (e.g., CT scans, MRI scans, X-rays) of a patient. Additional details regarding smart headset 2940 features and functionality are described in greater detail with reference to FIGS. 33-38 and 42.

Still referring generally to FIGS. 29-32, surgical tools 2910 and 2980 can include similar features and functionality instrument or tool 701 and driver 230. In particular, surgical tool 2910 can be fixedly coupled to an electronic device (e.g., 2920 or 2960) via a mounting device (e.g., 2930 or 2970). Surgical tool 2910 and 2980 can be configured to be aligned to anatomy of a body for inserting a surgical hardware (or medical device) a desired orientation through a three-dimensional insertion angle at a desired location (or point) on the anatomy of a body (or animal). For example, surface-based sensing or imaging technology (e.g., Light Detection and Ranging (“LIDAR”) technology, optical topographical imaging) may be used by smart headset 2940 to obtain data that can be used to create a 2D or 3D image map of or an anatomical surface (and/or edges) or profile associated with an anatomy of a body or a surgical tool (e.g., 2910 or 2980). In another example, a sensor of the surgical tool (e.g., geolocator, position sensor, and/or accelerometer) can transmit or communicate with the electronic device (e.g., 2920, 2940, and/or 2960). The use of LIDAR are described with further reference to International Application No. PCT/US2022/024683 filed Apr. 13, 2022, the entirety of which is incorporated by reference herein. In some implementations, surgical tool 2980 can include a plurality of physical elements or fiducial markers (sometimes referred to herein as “indicators” or “reflective element” or “fiducials” or the geometric shape) 2982 and 2984. In certain implementations, physical elements or fiducial markers 2982 and 2984 (e.g., indicators, fiducials, reflective elements) can be collected and analyzed by smart headset 2940 or electronic device 2920 determine the orientation of the surgical tool 2980 within the environment. That is, instead of receiving positioning information and environmental data from the electronic devices 2920 or 2960, the smart headset 2940 (individually or in combination with the electronic device 2920) can calculate the position and orientation of the surgical tool 2980 based on the physical elements or fiducial markers or geometric shapes. It should be understood that in other implementations, the smart headset 2940 does not require the use of indicators 2982 and 2984 and/or other physical elements or fiducial markers (or fiducials or geometric shapes) and/or environmental data. In some implementations, surgical tool 2910 can include a processing circuit and memory and can communicate positioning and orientation data to smart headset 2940 over a network. In the following embodiment, the surgical tool 2910 can include similar features and functionality described in detail with reference to electronic devices 2920 and 2960. Additional details regarding surgical tools 2910 and 2980 generally and determining the orientation of the surgical tool 2910 is described above in detail with reference to FIGS. 3-21.

Referring now to FIG. 29, an illustration of a surgical tool 2910, an electronic device 2920, and a smart headset 2940 in an environment 2900, according to example implementations. In some implementations, the environment 2900 can include a surgical tool 2910, an electronic device 2920, a mounting device 2930, a smart headset 2940, and a user 2950. In some implementations, the systems, and devices of environment 2900 (e.g., 2920, 2940) may be communicably and operatively coupled to each other over network 2902, that permits the direct or indirect exchange of data, values, instructions, messages, and the like (represented by the double-headed arrows in FIG. 29). As shown, the smart headset 2940 and electronic device 2920, mounted via mounting device 2930 to surgical tool 2910, can exchange environmental data, procedure information (e.g., desired three-dimensional insertion angle, desired location, anatomy, etc.), and other data, via network 2902, to allow the smart headset 2940 to generate and display graphical elements including visual indicia for orienting the surgical tool at the desired three-dimensional insertion angle at the desired location. In some implementations, the graphical elements can be superimposed (or overlayed) within the environment 2900.

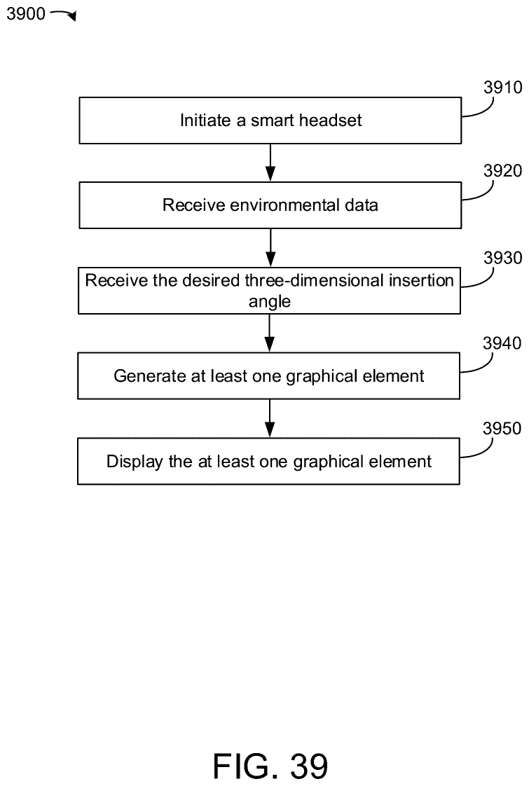

In various implementations, the smart headset 2940 and the electronic device 2920 can be a shared computing system configured to execute instructions in parallel or sequentially to accomplish a specific task. In particular, the shared computing system can employ processing power and resources from the smart headset 2940 and the electronic device 2920 to perform various tasks. For example, the electronic device 2920 may be configured to generate headset display interfaces (sometime referred to herein as “graphic elements”) and transmit the headset display interfaces to the smart headset 2940 for display. In another example, the smart headset 2940 may be configured to generate headset display interfaces (sometimes referred to herein as “graphic elements”) and display interfaces to the smart headset 2940 for display, in response to receiving environment data and a desired three-dimensional insertion angle from electronic device 2920 (e.g., FIG. 39). As should be understood, the shared computing system can be employed to utilize various resources of the electronic device 2920 and smart headset 2940 to collect, receive, transmit, and display information described in detail with reference to FIGS. 33-38. Additionally, the shared computing system can be employed to utilize various resources of the electronic device 2920 and smart headset 2940 to execute the various methods of FIGS. 39-41.

For example, the electronic device 2920 can be configured to collect orientation data of the surgical tool 2910 through sensors mounted on the tool via the mounting device 2930. That is, the sensors can provide continuous, real-time data on the tool's position and orientation relative to the surgical site. For example, the sensors can detect the tool's angular displacement, axial rotation, and insertion depth, providing a set of data points that describe the tool's exact spatial orientation. This collected data can be transmitted to the smart headset 2940 via network 2902. The smart headset 2940 can process this information and generate precise graphical elements such as directional arrows, alignment grids, and depth markers. These graphical elements can be superimposed onto the user's field of view through the headset's display. For example, the directional arrows can indicate the correct trajectory, the alignment grids can show the angular orientation, and the depth markers can display the insertion depth. This setup ensures the tool aligns with the desired three-dimensional insertion angle and target location.

In another example, the smart headset 2940 can be configured to receive detailed procedural information from the electronic device 2920, such as the desired three-dimensional insertion angle, the specific anatomical target location, and other relevant parameters. That is, the electronic device 2920 can store procedural data, including preoperative planning information and patient-specific anatomical models. For example, the surgeon can input the desired insertion angle and target coordinates into the electronic device 2920, which can then transmit this data to the smart headset 2940. The smart headset 2940 can use this information to generate visual indicia, such as insertion paths and target overlays, that guide the tool's orientation and positioning. These visual indicia can be displayed in the user's field of view, ensuring the surgical tool is aligned correctly with the target anatomy throughout the procedure.

In yet another example, the smart headset 2940 can update the visual display dynamically based on the user's movements and the tool's position. That is, as the user moves or the tool's orientation changes, the sensors on the tool can detect these changes and transmit updated data to the electronic device 2920. For example, if the tool deviates from the desired insertion path, the sensors can capture the deviation and send this information to the electronic device 2920. The electronic device 2920 can process the updated data and generate new graphical elements that reflect the current position of the tool. These updated graphical elements can then be transmitted to the smart headset 2940, which can adjust the visual display to show the new orientation and positioning of the tool. This real-time adjustment ensures that the user always has accurate and up-to-date visual guidance, maintaining the correct tool alignment and insertion angle.

In some implementations, the Apple Vision Pro can be used as the smart headset 2940 to enhance the surgical guidance system described in FIG. 29. The smart headset 2940 can be calibrated to the surgical environment to establish its position relative to the operating room and the patient. The smart headset 2940 can receive real-time data from the electronic device 2920 and the surgical tool 2910 via network 2902. This data can include the tool's orientation, position, and the desired three-dimensional insertion angle. Using its processors and augmented reality (AR) capabilities, the smart headset 2940 can generate and display graphical elements such as directional arrows, alignment grids, and depth markers directly in the surgeon's field of view. These graphical elements can be superimposed onto the real-world environment through the headset's displays, providing the surgeon with visual cues. The smart headset 2940 can use eye-tracking and gesture-recognition technologies to allow the surgeon to interact with the graphical elements without needing to touch any physical controls. Additionally, the smart headset 2940 built-in cameras and sensors can continually monitor the surgical tool's position, updating the visual guidance in real-time. The headset can operate using an integrated battery or external battery (e.g., connected in proximity to the headset, such as attached to and wired the headset or in a pocket of the surgeon attached providing power via a power cable to the headset), ensuring mobility and flexibility during the surgical procedure. The battery can provide power to the displays, sensors, and processing units, allowing the smart headset 2940 (e.g., Apple Vision Pro, Meta Quest 3, Google Glass) to function without being tethered to an external power source. This setup can maintain the precision and accuracy of the surgical tool's orientation and positioning throughout the procedure.

Generally, the processing circuits can implement and use generative AI (GAI or GenAI) in the surgical guidance system described in FIG. 29. The processing circuits can collect data from the sensors attached to the surgical tool 2910 and the environment 2900. This data can include spatial orientation, position, and real-time movement of the surgical tool. The generative AI model (e.g., executed by the processor of the smart headset 2940 or the sensors) can process this data to create models and graphical elements that assist in guiding the surgical tool to the desired three-dimensional insertion angle. The processing circuits can generate visual indicia such as trajectory paths, angular alignment markers, and insertion depth indicators. These graphical elements can be superimposed within the smart headset 2940's display, providing real-time visual guidance to the surgeon. The generative AI can adapt to changes in the tool's position and the user's movements by continually updating the graphical elements based on new data inputs.

For example, the generative AI model can predict the path for the surgical tool 2910 based on the current orientation and desired insertion angle. In another example, the AI model can adjust the visual indicia if the tool deviates from the planned path, providing corrective guidance to the surgeon. In yet another example, the generative AI can analyze patient anatomical data to customize the graphical elements, ensuring that the guidance is tailored to the characteristics of the patient's anatomy. The AI model can also incorporate feedback from the surgeon's eye movements or gestures detected by the smart headset 2940, allowing for hands-free adjustments of the visual guidance.

Generally, training and deploying the generative AI model can include a dataset including various surgical scenarios, tool orientations, and patient anatomical models can be collected. This dataset can be used to train the AI model using supervised learning techniques, where the model learns to generate graphical elements based on input data. The training process can include multiple iterations to refine the model's accuracy and performance. Once trained, the model can be deployed on the processing circuits of the smart headset 2940 and the electronic device 2920. Deployment can include integrating the AI model with the real-time data collection and processing systems to ensure seamless operation during surgical procedures. The model can be updated continually with new data to improve its predictive accuracy and adapt to different surgical environments and tool configurations.

Training the generative AI model can begin with the creation of a dataset that includes various types of surgical scenarios, multiple orientations of surgical tools, and a range of patient anatomical models. This dataset can be used for teaching the AI to recognize and generate accurate graphical elements. The AI model can be trained using supervised learning techniques, where it is provided with input data and the corresponding correct output. The training can include running the model through numerous iterations, each time adjusting the parameters to reduce errors and improve the model's ability to generate precise visual guidance. During each iteration, the model can be tested and validated to ensure it meets the desired performance standards.

Once the generative AI model is trained, it can be integrated into the processing circuits of the smart headset 2940 and the electronic device 2920. This integration can include configuring the AI model to work with real-time data from the surgical tool and the environment. The AI model can be deployed in a manner that allows it to receive continuous updates and new data, enhancing its accuracy and reliability over time. The deployment process can also include setting up mechanisms for the AI to learn from ongoing surgeries, allowing the AI model to adapt to new situations and improve its guidance capabilities. This continuous learning can help maintain the effectiveness of the AI model across a variety of surgical environments and tool configurations.

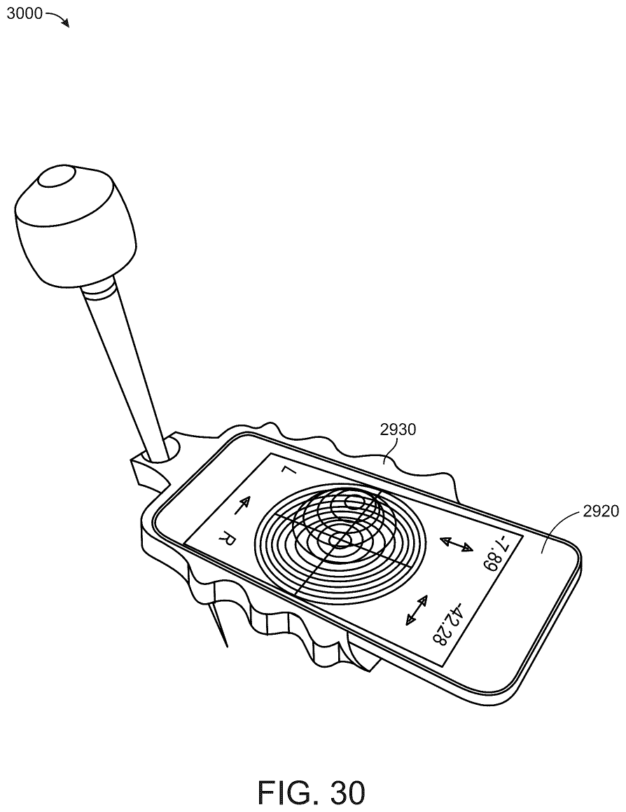

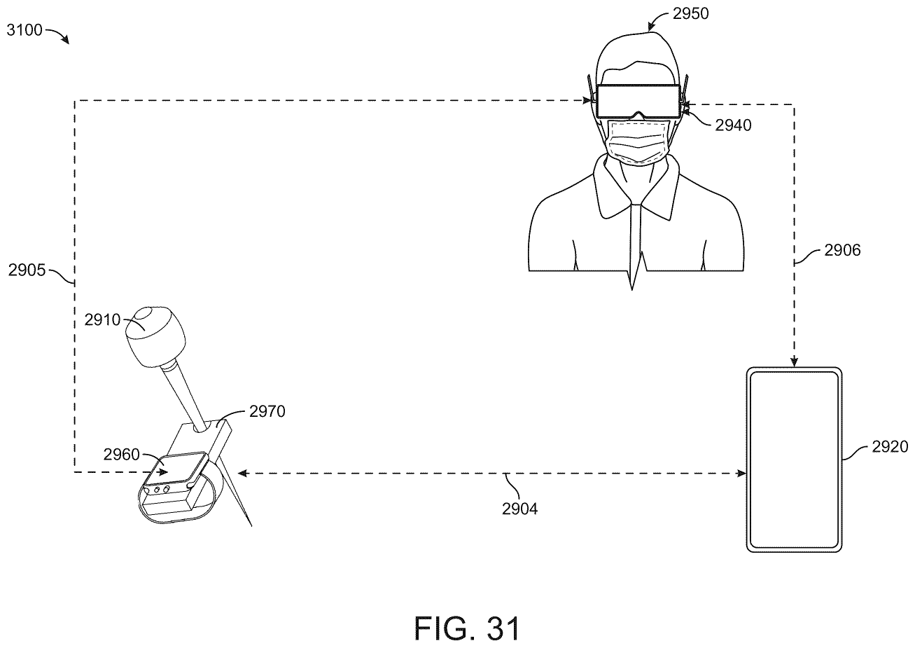

Referring now to FIG. 30, an illustration of a surgical tool 2910 and an electronic device 2920, according to example implementations. As shown the surgical tool 2910 and electronic device 2920 are fixedly coupled via mounting device 2930. The mounting device 2930 can fix the electronic device 2920 to surgical tool 2910 such that the orientation and position of the tool can be determine by the electronic device 2920 and/or smart headset 2940. Mounting device 2970 of FIG. 31 includes similar features and functionality as mounting device 2930 but instead is fixedly couples electronic device 2960 to surgical tool 2910. Additional details regarding the mounting device 2930 are described in detail with reference to FIG. 3A and FIG. 7, in particular attachment mechanism 308 and 700.

Referring now to FIG. 31, an illustration of a surgical tool 2910, multiple electronic devices 2920 and 2960, and a smart headset 2940 in an environment 3100, according to example implementations. In some implementations, the environment 3100 can include a surgical tool 2910, an electronic device 2920, an electronic device 2960, a mounting device 2970, a smart headset 2940, and a user 2950. In some implementations, the systems, and devices of environment 3100 (e.g., 2920, 2940, 2960) may be communicably and operatively coupled to each other over networks (e.g., 2904, 2905, 2906), that permits the direct or indirect exchange of data, values, instructions, messages, and the like (represented by the double-headed arrows in FIG. 31). As shown, the smart headset 2940 and electronic device 2920 (e.g., mobile phone, personal computer) can exchange environmental data, procedure information, and other data, via network 2905, to allow the smart headset 2940 to generate and display graphical elements including visual indicia for orienting the surgical tool at the desired three-dimensional insertion angle at the desired location. Additionally, the smart headset 2940 and electronic device 2960 (e.g., smart watch, smart IoT device) can also exchange environmental data, procedure information, and other data, via network 2905, to allow the smart headset 2940 to generate and display graphical elements including visual indicia for orienting the surgical tool at the desired three-dimensional insertion angle at the desired location. In some implementations, the electronic device 2920 and electronic device 2960 can also exchange environmental data, procedure information, and other data, via network 2904. As such, the electronic device 2920 and electronic device 2960 may relay data, via each other, to smart headset 2940. For example, if network 2906 is down or unavailable, electronic device 2920 may relay procedure information via electronic device 2960 to smart headset 2940. In another example, if network 2905 is down or unavailable, electronic device 2960 may relay procedure information via electronic device 290 to smart headset 2940. In some implementations, each network (e.g., 2904, 2905, 2906) can be the same type of network (e.g., Bluetooth, peer-to-peer, near field communication, Wi-Fi). In various implementations, each network (e.g., 2904, 2905, 2906) may be a different type of network.

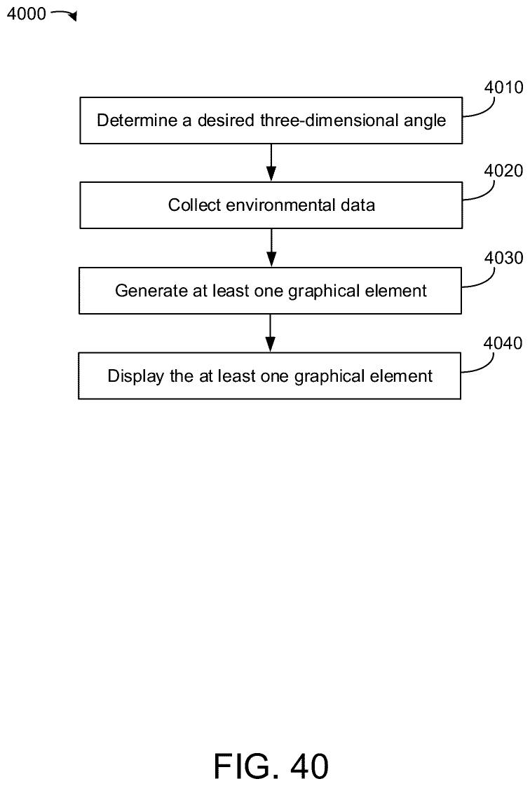

In various implementations, the smart headset 2940 and the electronic devices 2920 and 2960 can be a shared computing system configured to execute instructions in parallel or sequentially to accomplish a specific task. In particular, the shared computing system can employ processing power and resources from the smart headset 2940 and the electronic devices 2920 and 2960 to perform various tasks. For example, the electronic device 2920 may be configured to generate headset display interfaces and transmit the headset display interfaces to the smart headset 2940 for display, and the electronic device 2960, mounted to mounting device 2970, can collect orientation data of the surgical tool 2910 and transmit the orientation data to the smart headset 2940. In another example, the electronic device 2920 may be configured to collect orientation data from electronic device 2960 and in turn, generate headset display interfaces and transmit the headset display interfaces to the smart headset 2940 for display (e.g., FIG. 40). In yet another example, the smart headset 2940 may be configured to generate headset display interfaces and display interfaces to the smart headset 2940 for display, in response to receiving environment data and a desired three-dimensional insertion angle from electronic device 2920 and/or electronic device 2960. As should be understood, the shared computing system can be employed to utilize various resources of the electronic devices 2920 and 2960 and smart headset 2940 to collect, receive, transmit, and display information described in detail with reference to FIGS. 33-38. Additionally, the shared computing system can be employed to utilize various resources of the electronic devices 2920 and 2960 and smart headset 2940 to execute the various methods of FIGS. 39-41.

In various implementations, the watch, labeled as electronic device 2960, is configured to collect and transmit orientation data of the surgical tool 2910 to the smart headset 2940 within the environment 3100. The watch can be equipped with sensors such as gyroscopes and accelerometers to continually monitor the tool's angle, position, and movement during the surgical procedure. For example, the gyroscopic sensors can detect changes in the tool's orientation, while accelerometers can measure the dynamics of its movement. This collected data can be transmitted to the smart headset 2940 via network 2905, enabling the smart headset to generate and display precise graphical elements and visual indicia that guide the tool's positioning at the desired three-dimensional insertion angle. Additionally, the watch can interact with electronic device 2920 to relay data, ensuring continuous communication within the shared computing system. For example, if the direct network connection between the watch and the smart headset is unavailable, the watch can route the data through electronic device 2920, maintaining the data flow for accurate surgical guidance. This configuration allows the smart headset 2940 to utilize the data collection capabilities of the watch to enhance the precision of the surgical tool orientation process.

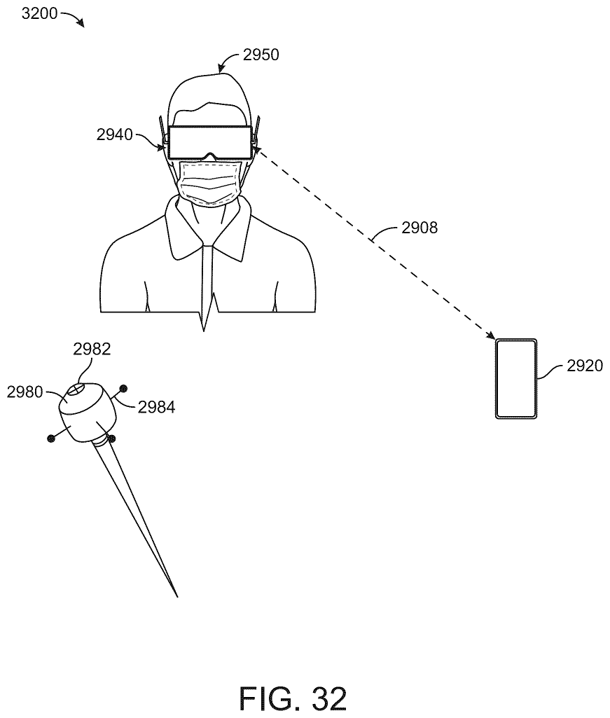

Referring now to FIG. 32, an illustration of a surgical tool 2980, an electronic device 2920, and a smart headset 2940 in an environment 3200, according to example implementations. In some implementations, the environment 3200 can include a surgical tool 2910, an electronic device 2920, and a user 2950. In some implementations, the systems, and devices of environment 3200 (e.g., 2920, 2940) may be communicably and operatively coupled to each other over a network (e.g., 2908), that permits the direct or indirect exchange of data, values, instructions, messages, and the like (represented by the double-headed arrows in FIG. 32). As shown, the smart headset 2940 and electronic device 2920 (e.g., mobile phone, personal computer) can exchange environmental data, procedure information, and other data, via network 2908, to allow the smart headset 2940 to generate and display graphical elements including visual indicia for orienting the surgical tool at the desired three-dimensional insertion angle at the desired location. In some implementations, the smart headset 2940 may determine orientation and position of the surgical tool 2980 based on physical elements or fiducial markers (e.g., indicators 2984, indicators 2982, sometimes referred to herein as “indicators”) coupled to surgical tool 2980. In some implementations, indicators 2984 may be arranged around the top (or head) or the surgical tool 2980. For example, there can be four indicators 2984 arranged around the head of surgical tool 2980. In another example, there can be six or eight indicators 2984 arranged around the head of surgical tool 2980. In particular, the indicators 2984 can be, but is not limited to, 1-50 millimeters, 1-5 centimeters, or 0.25-2 inches in length, and can be, but is not limited to, 1 nanometer-100 million nanometers, 1-10,000 micrometers in diameter. Additionally, the indicators 2984 can be coupled to the head and can be perpendicular to the side of the head. In some implementations, the indicators may be an acute angle to the side of the head or may be an obtuse angle to the side of the head. In some implementations, indicator 2982 can include one or more lines printed or coupled to the top of the head (where the head can be flat or curved). For example, the lines can form an X with 4 right angles. In another example, the lines can form two acute angles and two obtuse angles.

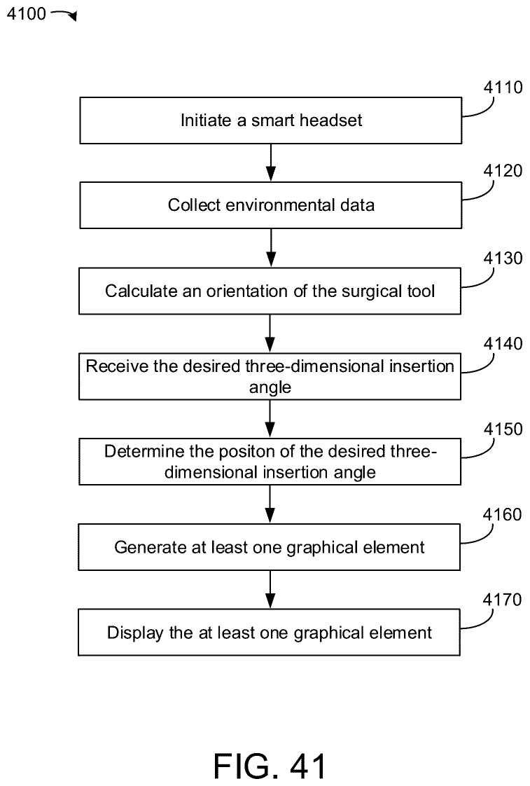

In some implementations, there may be greater than two lines or less than two lines. In various implementations, the smart headset 2940 and the electronic device 2920 can be a shared computing system configured to execute instructions in parallel or sequentially to accomplish a specific task. In particular, the shared computing system can employ processing power and resources from the smart headset 2940 and the electronic device 2920 to perform various tasks. For example, the electronic device 2920 may be configured to generate headset display interfaces and transmit the headset display interfaces to the smart headset 2940 for display, in response to receiving orientation data (e.g., in real-time, or near real-time) from smart headset 2940 based on the indicators (e.g., 2982 and 2984) of surgical tool 2980. In another example, smart headset 2940 can analyze the indicators of surgical tool 2980 to determine orientation and generate the graphical elements based on the orientation of the surgical tool 2980 (e.g., FIG. 41). In the following example, electronic device 2920 may transmit anatomy information of the body or portion of the body associated with the procedure such that the smart headset 2940 can use that information in combination with collected environment data to generate graphical elements. As should be understood, the shared computing system can be employed to utilize various resources of the electronic device 2920 and smart headset 2940 to collect, receive, transmit, and display information described in detail with reference to FIGS. 33-38. Additionally, the shared computing system can be employed to utilize various resources of the electronic device 2920 and smart headset 2940 to execute the various methods of FIGS. 39-41.

In various other implementations, the smart headset 2940 and the electronic device 2920 can be partially or fully integrated as one device or system, such as all being integrated as part of the smart headset 2940, and configured to execute instructions in parallel or sequentially to accomplish a specific task. In particular, such an integrated device or system can employ processing power and resources to provide all of the functionality of both the electronic device 2920 and the smart headset 2940, such as to generate headset display interfaces for display. In another example, the smart headset 2940 can analyze the indicators of the surgical tool 2980 to determine orientation and generate the graphical elements based on the orientation of the surgical tool 2980 (e.g., FIG. 41). In still other implementations, the integrated device or system implemented as smart headset 2940 may generate the graphical elements, such as desired surgical tool position, a tool icon, in various positions and orientations, a lock icon, navigational objects, and other indicators, as needed, and without the use of indicators or physical elements or fiducial markers. The integrated device or system can be employed to utilize various resources of the electronic device 2920 and smart headset 2940 to collect, receive, transmit, and/or display information described in detail with reference to FIGS. 33-38. Additionally, the integrated device or system can be employed to utilize various resources of the electronic device 2920 and smart headset 2940 to execute the various methods of FIGS. 39-41.

Referring now generally to FIGS. 33-38, illustrations of various individual views of the smart headset 2940 of FIG. 42, according to example implementations. Generally, various views can be a combination of headset display interfaces (e.g., on headset display 3301) overlaid on environment 2900 during a session (e.g., upon initiation of the smart headset 2940). The headset display interfaces can include a plurality of interfaces and objects overlaid on environment 2900 such that an individual (e.g., a user or human operator) can provide biological data (e.g., stress level, heart rate, hand geometry, facial geometry, psyche, and so on) and/or behavioral data (e.g., haptic feedback, gesture, speech pattern, movement pattern (e.g., hand, food, arm, facial, iris, and so on), intangible feedback (e.g., selection of intangible content displayed on smart headset 2940), response to stimuli, and so on) to interact with the plurality of interfaces, objects, and/or environment 2900. For example, an individual may complete an action (e.g., lock, settings) by selecting an object overlaid on environment 2900 (e.g., intangible object) with a hand gesture (e.g., point at object). In another example, an individual may complete an action by selecting at an object overlaid on environment 2900 with an eye movement (e.g., look at object). In yet another example, an individual may provide their heart rate that may indicate a level of stress. In yet another example, an individual may touch the surgical tool 2910 or smart headset 2940 (e.g., haptic feedback) to provide input when completing a task (e.g., orientating the surgical tool, initiating the smart headset 2940 and so on). In various implementations, an individual may also receive notifications (e.g., alerts, requests, status indicators, and so on) on the headset display interface, for example, indicating an action to perform and/or session information (e.g., anatomy, planes, vectors). In various implementations, the smart headset 2940 may be paired (e.g., Bluetooth, NFC, wireless connection, wired connection, and so on) with electronic devices 2920 and 2960 and/or any computing system described herein.

Still referring generally to FIGS. 33-38, the smart headset 2940 can include a headset display 3301. The headset display can be any suitable see-through display (sometimes referred to as a “transparent display”) that utilizes any suitable technique to display a graphical user interface on the headset display. Various see-through displays can include, for example, a transparent screen substrate fused with a liquid crystal technology (LCD), a light field display (LFD), a head-up display (HUD), a transparent screen substrate fused with an organic light-emitting diode display (OLED), a transparent electroluminescent display (TASEL), and so on. Various techniques can include, for example, “Curved Mirror” (or “Curved Combiner”) based, “Waveguide” (or “light-guide”) based, and so on. In various implementations, the smart headset 2940 may be of varying sizes, for example, a helmet, a virtual reality headset, an augmented reality headset, smart glasses, a hat, a headdress, and/or any type of headgear. In some implementations, the headset display may be opaque (or a percentage opaque, sometimes referred to as “translucent”). Thus, the headset display and/or smart headset 2940 is not limited to any specific combination of hardware circuitry and software.

Still referring generally to FIGS. 33-38, the display 3301 may display a tool icon (e.g., 3302) configured to allow a user of the smart headset 2940 to customize the experience when interacting with the smart headset 2940. In various implementations, when the tool icon 3302 is selected via a biological or behavioral action, it can allow a user to set specific arrangement and/or settings (e.g., colors, size, preferences, authentication procedure, and so on) when the graphical user interface (collectively referred to herein as the “the headset display interface”) are shown on the headset display (e.g., 1301). For example, if a user (e.g., 2950) is color blind, they may configure, via the tool icon 3302, a smart headset setting such that any notifications displayed on the headset display 3301 are not green or red. In another example, if a user has trouble with sight out of one eye, they may configure, via the tool icon 3302, a smart headset setting such that one side of the headset display is favored over another (e.g., for showing objects/content). In yet another example, a user could configure, via the tool icon 3302, the size of text/objects of the headset display interface.

In some implementations, the smart headset 2940 (FIGS. 33-38) may include one or more processing circuits that when executed can generate various graphical user interfaces (e.g., visual indicia, objects, content). The smart headset 2940 can include one or more processors (e.g., any general purpose or special purpose processor), and include and/or be operably coupled to one or more transitory and/or non-transitory storage mediums and/or memory devices (e.g., any computer-readable storage media, such as a magnetic storage, optical storage, flash storage, RAM, and so on) capable of providing one or more processors with program instructions. Instructions can include code from any suitable computer programming language. The one or more processing circuits that when executed can generate various graphical user interfaces. In some implementations, the smart headset 2940 may vary in size and may be integrated with various input/output devices 4240 (e.g., sensors, IoT devices, cameras). In various implementations, smart headset client application 4238 of FIG. 42 can be configured to provide the graphical user interfaces (e.g., personalized views) to the smart headset 2940 to facilitate improved content presentation to various users of a session (e.g., doctors, and so on). Additional details relating to the various views of the smart headset 2940 are provided herein with respect to FIGS. 33-38.

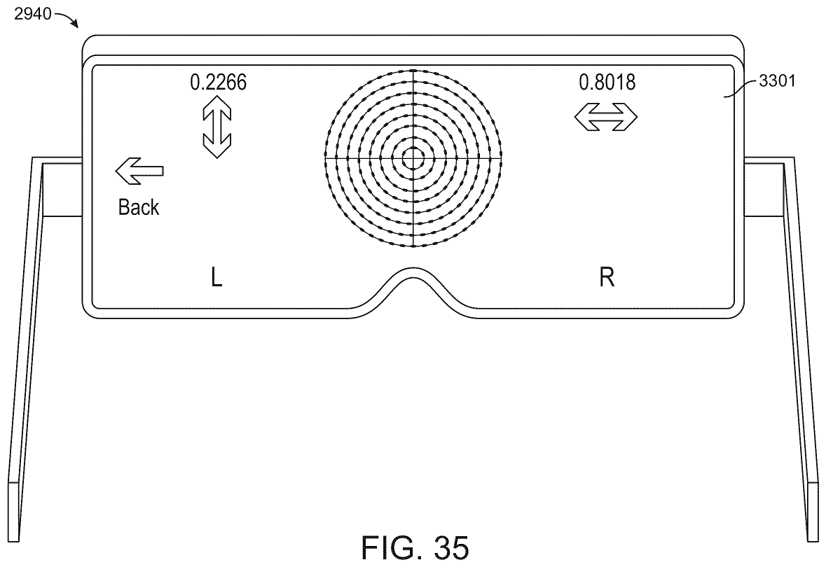

Referring now to FIGS. 33-35 in more detail. FIGS. 33-35 illustrate views of the smart headset 2940 of FIGS. 29-32. FIG. 33 is shown to include a plurality of graphical elements (also referred to as “graphical interface objects”) displayed on headset display 3301 including concentric circles 3309A and 3309B, a tool icon 3302, and various orientation and positioning information. In some arrangements, the graphical interface objects or elements can be visual indicia for orienting surgical tool 2910. For example, the orientation and positioning information can be received from electronic device 2920 mounted to surgical tool 2910 (e.g., FIG. 29). In another example, the orientation and positioning information can be received from electronic device 2960 mounted to surgical tool 2910 (e.g., FIG. 31). In yet another example, the orientation and positioning information can be collected and analyzed by smart headset 2940 (e.g., FIG. 32). Additionally, a user may be wearing the smart headset 2940 and concentric circle 3309B may move as the user 2950 moves the surgical tool 2910 (or 2980) to orient it at a desired three-dimensional insertion angle at a desired location within an environment. In particular, concentric circle 3309B can be the current position of the surgical tool 2910 (e.g., coupled to the electronic device 2920 (FIG. 29) or 2960 (FIG. 31), or determine by smart headset 2940 (FIG. 32)), whereas concentric circle 3309A can be the desired position (i.e., desired three-dimensional insertion angle at a desired location). Accordingly, as shown with reference to FIG. 35, when the concentric circle 3309B overlaps concentric circle 3309A then the surgical tool 2910 can be oriented at the approximate insertion angle and location (e.g., on a portion of an anatomy).

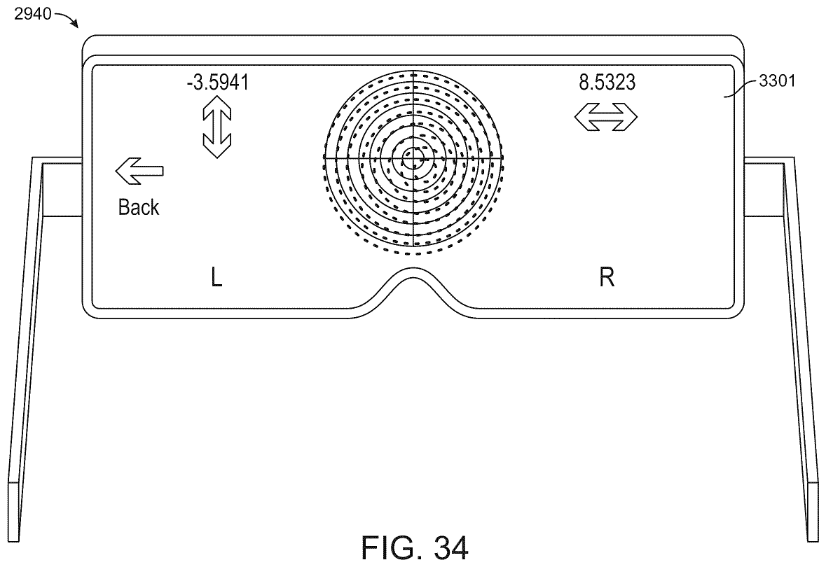

FIG. 34 is also shown to include a plurality of graphical elements displayed on headset display 3301 including concentric circles and various orientation and positioning information. As shown, the graphical elements indicating the positioning (e.g., Y (or perpendicular) axis: −3.5941, X (or horizontal) axis: 8.5323) can update (e.g., compared to FIG. 33) as the operator of the surgical tool 2910 moves the surgical tool 2910 throughout the environment. Additionally, the graphical elements including the current concentric circles (e.g., dashed lines) can also update as the operator of the surgical tool 2910 moves the surgical tool 2910 throughout the environment.

FIG. 35 is also shown to include a plurality of graphical elements displayed on headset display 3301 including concentric circles and various orientation and positioning information. As shown, the graphical elements indicating the positioning (e.g., Y (or perpendicular) axis: −0.2266, X (or horizontal) axis: 0.8018) can update (e.g., compared to FIGS. 33-34) as the operator of the surgical tool 2910 moves the surgical tool 2910 throughout the environment. Additionally, the graphical elements including the current concentric circles (e.g., dashed lines) can also update as the operator of the surgical tool 2910 moves the surgical tool 2910 throughout the environment. As shown, when the concentric circles overlap and the positioning can be approximately 0 (e.g., +/−1.5 cm, +/−2.5 mm, +/−1 inch) then the surgical tool 2910 can be oriented at the approximate insertion angle and location (e.g., on a portion of an anatomy).

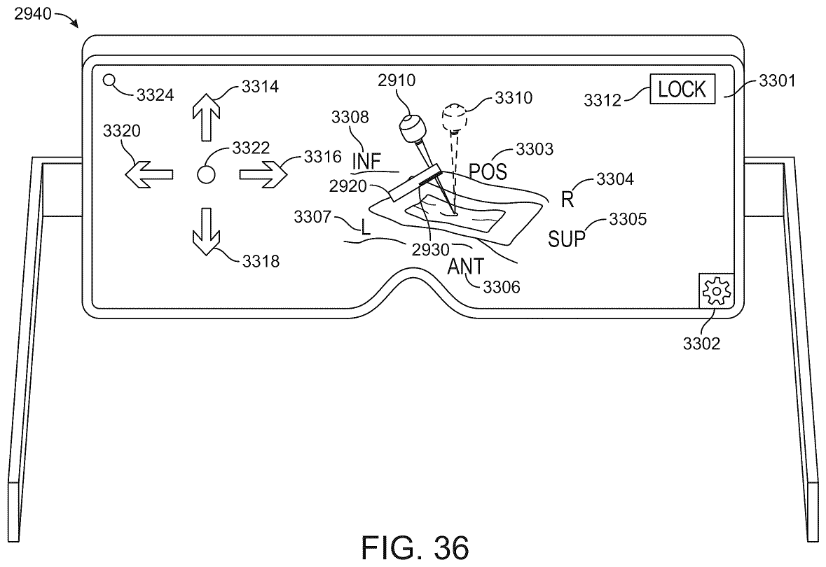

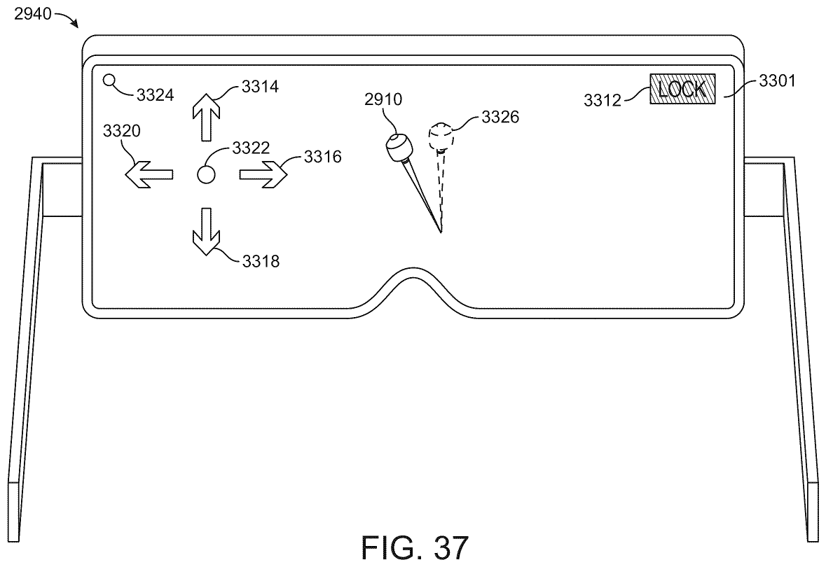

Referring now to FIGS. 36-38 in more detail. FIGS. 36-38 illustrate views of the smart headset 2940 of FIGS. 29-32. FIG. 36 is shown to include a plurality of graphical elements (also referred to as “graphical interface objects”) displayed on headset display 3301 including a desired surgical tool position 3310, a tool icon 3302, and various orientation and positioning information (e.g., 3303, 3304, 3305, 3306, 3307, 3308), a lock icon 3312, navigational objects (e.g., 3314, 3316, 3318, 3320, 3322), and an indicator 3324. In some arrangements, the graphical interface objects or elements can be visual indicia for orienting surgical tool 2910. Also shown, an environment including a surgical tool 2910, electronic device 2920, a mounting device 2930, and a human anatomy (e.g., back) where the surgical tool 2910 can be to be used to perform an operations. In some arrangements, that the graphical elements can be superimposed within the environment, via a translucent display of smart headset 2940 as shown with reference to FIGS. 36-38. In various arrangements, that the graphical elements can be displayed on the smart headset 2940, via an opaque display of smart headset 2940. In some arrangements, the smart headset 2940 can be switched (e.g., upon the user selecting a graphical element (e.g., selecting graphical element 3322)) from opaque mode to translucent mode within an operations or based on the type of operations. It should be understood, the user can provide intangible feedback by completing a selection of graphical elements (or objects) shown on the display of the smart headset 2940. The selection can be within the environment (e.g., within empty space such as air) such that the user may hover over and make a selection (e.g., touch) of a superimposed object and/or element. Accordingly, the user of a smart headset 2940 before, during, or after, an operation or activity can complete selections of objects based on the retrieving and/or receiving (e.g., by a processor) data from various input/output devices indicating a selection occurred (e.g., raise hand and point, wave hand, kick foot, nod head, and so on, with reference to FIGS. 33-38 shown above).

The desired surgical tool position 3310 can be generated based on the desired three-dimensional insertion angle at a desired location within an environment. The orientation and positioning information (e.g., 3303, 3304, 3305, 3306, 3307, 3308) presented on the smart headset 2940 can be updated as the user moves throughout the environment (e.g., such that the user always knows the anterior, inferior, posterior, superior, left, and right positions of the anatomy). The display 3301 may display a lock icon 3312 configured to allow a user of the smart headset 2940 to lock the position of the desired surgical tool position 3310. For example, during setup or during surgery a doctor may desire to change the desired insertion angle and position of the desired surgical tool position 3310. Upon selection of the lock icon the doctor can lock the desired surgical tool position 3310 within the environment such that as the user moves throughout the environment the desired surgical tool position 3310 will not change or update.

The display 3301 may display navigational objects (e.g., 3314, 3316, 3318, 3320, 3322) configured to allow a user of the smart headset 2940 to customize the experience when interacting with the smart headset 2940. In various implementations, when one or more navigational objects are selected by the user can navigate within the presented graphical user interface (e.g., select different styles of the desired surgical tool position 3310, select the type of operation, perform smart headset initiation, add, modify, or delete stored data on the smart headset 2940, and so on. As such, when the user selects a navigational object via a biological or behavioral action, it can allow a user to set and adjust the smart headsets 2940 arrangement and/or settings. The indicator 3324 (e.g., notifications) can be include different colors or designs based on the location of the surgical tool 2910 compared to the desired surgical tool position 3310. For example, when the surgical tool 2910 can be more than +/−5 inches away from the desired location (or position) indicator 3324 may be red, when the surgical tool 2910 can be less than +/−5 inches away from the desired location (or position) indicator 3324 may be orange, when the surgical tool 2910 can be at approximately (e.g., +/−1.5 cm, +/−2.5 mm) at the desired location (or position) indicator 3324 may be yellow, and when the surgical tool 2910 can be at approximately (e.g., +/−1.5 cm, +/−2.5 mm) at the desired insertion angle, indicator 3324 may be green.

FIGS. 37-38 is shown to include a plurality of graphical elements (also referred to as “graphical interface objects”) displayed on headset display 3301 including a desired surgical tool position of virtual surgical tool 3326, a lock icon 3312, navigational objects (e.g., 3314, 3316, 3318, 3320, 3322), and an indicator 3324. As shown, the lock icon 3312 can be highlighted or filled in indicating the desired surgical tool position of virtual surgical tool 3326 can be locked. The desired surgical tool position of virtual surgical tool 3326 includes similar features and functionality as desired surgical tool position 3310 of FIG. 36. However, as shown with reference to FIG. 38, the desired surgical tool position of virtual surgical tool 3326 can include a guideline 3330 for guiding the user of the surgical tool 2910 to the desired location and desired insertion angle.

Referring specifically to FIG. 36, the smart headset 2940 can include a detailed graphical interface on display 3301 designed to provide visual guidance for the user during surgical procedures. The display 3301 can include multiple graphical elements such as the desired surgical tool position 3310 and the tool icon 3302. These elements can indicate the exact positioning and orientation required for the surgical tool 2910. The display can also include various orientation and positioning markers such as POS 3303, R 3304, SUP 3305, ANT 3306, L 3307, and INF 3308, which can help the user understand the anatomical directions relative to the tool's position. The lock icon 3312 can be an interactive element that allows the user to lock the desired surgical tool position 3310, ensuring that the indicated position remains constant even as the user or the tool moves. This feature can be particularly useful for maintaining accuracy during the procedure. Navigational objects like 3314, 3316, 3318, 3320, and 3322 can be displayed to enable the user to navigate through different settings and modes within the graphical user interface. For example, these objects can allow the user to adjust the tool's orientation, switch between different operation modes, or modify the display settings.

The indicator 3324 can provide real-time feedback on the tool's position relative to the desired location. For example, the color of the indicator can change based on the tool's proximity to the target position, with specific colors representing different distances or angles. This visual feedback can help the user make necessary adjustments to achieve the precise insertion angle. The smart headset 2940 can superimpose these graphical elements within the user's field of view through either a translucent or opaque display. This flexibility can allow the user to choose the most suitable display mode for their needs. Additionally, the headset can switch between modes based on the user's selection or the type of operation being performed. The user can interact with the graphical elements through intangible feedback mechanisms such as gestures, eye movements, or other biometric inputs, allowing for hands-free operation and seamless interaction during the procedure.

Referring specifically to FIG. 38, the smart headset 2940 can include a detailed graphical interface on display 3301 designed to provide visual guidance for the user during surgical procedures. The display 3301 can include multiple graphical elements such as the virtual surgical tool 3326 and the guideline 3330. These elements can assist in indicating the exact positioning and orientation required for the surgical tool 2910. The virtual surgical tool 3326 can represent the target placement of the actual surgical tool within the environment. For example, the virtual surgical tool can help the user visualize the correct insertion angle and depth before performing the actual procedure.

The guideline 3330 can serve as a visual trajectory guide for the surgical tool 2910. That is, the guideline can provide a reference path that the user should follow to achieve the desired insertion angle and depth. For example, the guideline can be a dashed line that extends from the virtual surgical tool 3326 to the target insertion point, helping the user align the tool correctly. The lock icon 3312 can be an interactive element that allows the user to lock the position of the virtual surgical tool 3326. That is, by selecting the lock icon, the user can ensure that the virtual tool position remains fixed even as the user or the tool moves within the environment. For example, this feature can be useful during setup or adjustments, allowing the user to maintain the correct position of the tool without needing to recalibrate constantly. The lock icon can provide stability in the virtual display, ensuring the accuracy of the procedure.

Referring generally to FIGS. 39-41, method 3900 relates to a method for orienting a surgical tool at a desired three-dimensional insertion angle at a desired location within an environment for use in installing a medical device by using and displaying at least one graphical element. The method can be configured to initiate a smart headset to be calibrated to the environment so that the position of the smart headset (e.g., including a processing circuit) can be known relative to the environment when the smart headset moves in the environment. That is, the method can include initiating a smart headset calibration process to establish the headset's positional awareness within the environment. For example, the smart headset can determine its relative position as it moves within the operating environment. The smart headset can be configured to receive from an electronic device communicatively coupled to the smart headset, environmental data indicating the position of the surgical tool within the environment. That is, the smart headset can collect positional data of the surgical tool from an external electronic device to understand the tool's current location. For example, the environmental data can provide real-time updates on the tool's position. Additionally, the smart headset can be configured to receive from the electronic device, the desired three-dimensional insertion angle. That is, the smart headset can obtain the insertion angle required for the procedure from the electronic device. For example, the angle data can be transmitted to guide the placement of the surgical tool. The smart headset can be configured to generate at least one graphical element including visual indicia for orienting the surgical tool at the desired three-dimensional insertion angle at the desired location. That is, the smart headset can create graphical overlays that visually guide the user in positioning the tool correctly. For example, the visual indicia can include lines and shapes that represent the target insertion path. Furthermore, the smart headset can be configured to display the at least one graphical element superimposed within the environment. That is, the smart headset can project the graphical elements onto the real-world environment to assist the user in visualizing the insertion path. For example, the graphical elements can appear as augmented reality overlays on the headset's display.

In some implementations, the processing circuits can be configured such that the visual indicia includes a virtual tool for orienting the surgical tool at the desired location and the three-dimensional insertion angle. That is, the processing circuits can generate a virtual representation of the surgical tool to aid in precise orientation. For example, the virtual tool can mimic the actual tool's movements to verify accurate placement. Method 3900 can also be configured such that the visual indicia further include a three-dimensional vector including a guideline indicating a trajectory of the virtual tool. That is, the visual indicia can incorporate a vector line that visually guides the expected path of the tool. For example, the guideline can help the user align the tool correctly along the intended trajectory. The smart headset can be configured to generate interactive elements for interacting with the smart headset and to display the interactive elements superimposed within the environment. That is, the smart headset can produce elements that users can interact with to modify or adjust the tool's positioning. For example, these elements can include buttons or sliders that can be visible in the augmented reality view.

In some implementations, the processing circuits can be configured to receive an instruction from an individual operating the smart headset via an input device of the smart headset. That is, the processing circuits can accept commands from the user through various input methods integrated into the headset. For example, the input device can include voice commands, touch sensors, or gesture recognition. The smart headset can be configured to lock the virtual tool superimposed within the environment, such that the virtual tool remains stationary (e.g., does not move, does not rotate, does not shift, and/or does not drift) at the desired location and the three-dimensional insertion angle as the smart headset changes positions within the environment.

That is, the headset can fix the virtual tool in place, maintaining its position and angle despite any movements of the headset. For example, even if the user moves their head, the virtual tool can stay aligned at the designated insertion point. The instruction from the individual can be at least one of an eye movement (e.g., blinking, gaze direction, eye tracking), a gesture (e.g., hand wave, finger point, swipe motion), an auditory pattern (e.g., voice command, clap, whistle), a movement pattern (e.g., walking, head nod, arm raise), haptic feedback (e.g., vibration, pressure, touch), a biometric input (e.g., fingerprint, facial recognition, retinal scan), intangible feedback (e.g., ambient light change, temperature variation, sound intensity), or a preconfigured interaction (e.g., button press, pre-set sequence, programmed shortcut). For example, the user can lock the tool position using a hand gesture or voice command.

In some implementations, the visual indicia can be configured to include concentric circles indicating thresholds of the three-dimensional insertion angle of the surgical tool. That is, the visual indicia can display circles that represent acceptable ranges for the insertion angle to guide the user. For example, the circles can help the user understand if the tool can be aligned within the required angular limits. The concentric circles can be configured to include a first set of concentric circles indicating the orientation of the surgical tool at the desired location based on the three-dimensional insertion angle and a second set of concentric circles indicating a live orientation of the surgical tool. That is, the first set of circles can show the target orientation, while the second set can display the current orientation of the tool. For example, this can help the user make real-time adjustments to align the tool correctly. The electronic device can be calibrated to the surgical tool to indicate the live orientation of the surgical tool. That is, the electronic device can adjust its settings based on the tool's position to provide accurate orientation data. For example, calibration can verify that the real-time data reflects the actual tool orientation.

In some implementations, the environmental data can be configured to include orientation data of the surgical tool, and the smart headset can be configured to continually receive the environmental data from the electronic device in real-time. That is, the smart headset can collect ongoing orientation data to monitor the tool's position continually. For example, the real-time data can help in maintaining the correct tool alignment throughout the procedure. In response to continually receiving the environmental data, the smart headset can be configured to automatically update the at least one graphical element superimposed within the environment in real-time. That is, the graphical elements can change dynamically based on the new data received. For example, this can help the user adjust the tool position quickly and accurately.

In some implementations, the smart headset can be configured to include a gyroscope, and generating and displaying the at least one graphical element can be based on continually collecting orientation data of the smart headset in real-time by the gyroscope. That is, the gyroscope can gather orientation data to assist in creating accurate graphical elements. For example, the data collected by the gyroscope can help maintain the stability of the virtual overlays. In response to continually collecting the orientation data of the smart headset, the smart headset can be configured to automatically update the at least one graphical element superimposed within the environment in real-time. That is, the headset can ensure that the visual aids remain aligned with the user's view. For example, this can provide a consistent and reliable visual guide for the procedure.

In some implementations, the smart headset can be configured to capture additional environmental data of the environment via an input device of the smart headset. That is, the smart headset can gather more information about the surroundings to improve accuracy. For example, capturing environmental data can include scanning the room or identifying obstacles. In some implementations, the input device can be at least one of a camera, sensor, or internet of things (IoT) device. That is, various input devices can be used to collect the data. For example, cameras can provide visual data, while sensors can detect physical conditions. The additional environmental data can be configured to include orientation data of a portion of a body, indicating at least one of an axial plane, coronal plane, or a sagittal plane associated with the anatomy of the portion of the body. That is, the data can help in understanding the body's position relative to the tool. For example, knowing the axial, coronal, or sagittal planes can assist in accurate tool placement.

In some implementations, the smart headset can be configured to determine the orientation of the portion of the body within the environment based on inputting the orientation data into a machine learning algorithm and receiving an output prediction indicating the orientation of the portion of the body within the environment. That is, machine learning algorithms can analyze the orientation data to predict the body's position. For example, the algorithm can process complex data points to provide accurate orientation predictions. Generating the at least one graphical element including the visual indicia for orienting the surgical tool at the desired location can be further based on the orientation of the portion of the body within the environment. That is, the body's orientation data can influence how the visual aids can be created. For example, this can ensure the tool's path can be correctly aligned with the body structure. The smart headset can be configured to generate visual indicator elements indicating the orientation of the portion of the body within the environment and display the visual indicator elements superimposed within the environment. That is, the headset can create visual markers to show the body's orientation. For example, these markers can help the user align the tool with anatomical landmarks.

In some implementations, the surgical tool can be one of a gear shift probe, a pedicle probe, a Jamshidi needle, an awl, a tap, a screw inserter, a drill, or a syringe, and the environmental data can include planning data for performing an operation at the desired location using the surgical tool. That is, the method can support various types of surgical tools for different procedures. For example, each tool type can have planning data to guide its use. The smart headset can be configured to receive and store diagnostic images of a portion of a body. That is, the headset can handle images that aid in the surgical process. For example, storing diagnostic images can provide reference visuals for the user. In some implementations, generating the at least one graphical element can be further based on the diagnostic images of the portion of the body. That is, the diagnostic images can enhance the accuracy of the visual aids. For example, they can help in creating overlays that match the patient's anatomy.

In some implementations, the smart headset can be configured such that the environmental data includes one or more of positional data of the environment, body features of a user of the smart headset, and physical elements or fiducial markers of the surgical tool. That is, the environmental data can cover multiple aspects of the operating environment. For example, positional data can help in mapping the surroundings, body features can assist in understanding the user's interaction with the tool, and physical elements or fiducial markers of the tool can verify proper alignment.

In some implementations, the method can be configured such that the surgical tool can be one of a pedicle screw, an interbody cage, a stent, a pin, a rod, or a graft, and wherein the environmental data includes planning data for inserting the surgical tool. That is, the method can support the use of various surgical tools required for different types of implants or repairs. For example, planning data can provide guidance for each tool type, ensuring proper insertion techniques.

In some implementations, the method can be configured such that the surgical tool can be one of a pedicle screw, an interbody cage, a stent, a pin, a rod, or a graft, and wherein the environmental data includes planning data for inserting the surgical tool. That is, the method can accommodate different surgical tools needed for various procedures, with each tool having its own set of planning data. For example, this can guide the use of tools like pedicle screws or stents accurately.

In some implementations, the method can be configured such that the surgical tool can be one of a pedicle screw, an interbody cage, a stent, a pin, a rod, or a graft, and wherein the environmental data includes planning data for inserting the surgical tool. That is, the method can be adaptable to different types of surgical tools, providing relevant planning data for each tool to verify correct usage. For example, the planning data can include instructions and visual guides for inserting items like pedicle screws or rods.

In some implementations, the method can be configured such that the surgical tool can be one of a pedicle screw, an interbody cage, a stent, a pin, a rod, or a graft, and wherein the environmental data includes planning data for inserting the surgical tool. That is, the method can be designed to work with a range of surgical tools, with planning data available for each type to aid in their precise insertion. For example, the planning data can guide the user on how to properly position and insert tools like pins or grafts.

Generally, method 4000 relates to a method for orienting a surgical tool at a desired three-dimensional insertion angle at a desired location within an environment for use in installing a medical device by using and displaying at least one graphical element can be configured to initiate a smart headset to be calibrated to the environment so that the smart headset knows its position relative to the environment when the smart headset moves in the environment. That is, the method can involve calibrating a smart headset to understand its spatial location as it navigates the environment. For example, the smart headset can utilize sensors to determine its position and orientation within an operating room. The processing circuits can be configured to collect environmental data of the surgical tool within the environment using physical elements or fiducial markers of the surgical tool that can be located at the desired location. That is, the processing circuits can gather information about the tool's position and physical attributes within the environment. For example, the tool can have markers or sensors that relay its location to the smart headset. The processing circuits can be configured to calculate an orientation of the surgical tool based on collecting the physical elements or fiducial markers of the surgical tool. That is, the processing circuits can determine the tool's alignment and angle based on the collected data. For example, the circuits can analyze data points to establish the tool's spatial orientation. The smart headset can be configured to receive the desired three-dimensional insertion angle and determine the position of the desired three-dimensional insertion angle at the desired location. That is, the smart headset can obtain the required insertion angle and translate it to a location within the environment. For example, the headset can use this angle to guide the placement of the tool during surgery. The smart headset can be configured to generate the at least one graphical element including visual indicia for orienting the surgical tool at the desired three-dimensional insertion angle at the desired location. That is, the smart headset can create visual cues that help align the tool correctly. For example, the graphical elements can include arrows or lines that show the optimal path for the tool. Furthermore, the smart headset can be configured to display the at least one graphical element superimposed within the environment. That is, the headset can overlay these visual elements onto the real-world view. For example, the surgeon can see the insertion path directly on their display.

In some implementations, the software (or executable code) of the processing circuits can recognize the shape of the object without delineated or identifiable markers. That is, the processing circuits can be configured to collect environmental data (e.g., geometric shape, size, orientation) of the surgical tool within the environment, which can be used to determine its positioning and alignment relative to the desired insertion angle. Additionally, the processing circuits can gather information about the physical characteristics and spatial relationship of the surgical tool to the surrounding environment. For example, the processing circuits can analyze the shape and orientation of the tool in real-time, using this data to update and refine the graphical elements displayed by the smart headset.

In some implementations, the visual indicia can include a virtual tool for orienting the surgical tool at the desired location and the desired three-dimensional insertion angle. That is, the headset can display a virtual representation of the tool to assist with orientation. For example, the virtual tool can show how the actual tool should be positioned. The visual indicia can further include a three-dimensional vector including a guideline indicating a trajectory of the virtual tool. That is, the visual elements can also include a vector that shows the tool's expected path. For example, the vector can help the user align the tool along the intended trajectory. The smart headset can be configured to generate interactive elements for interacting with the smart headset and display the interactive elements superimposed within the environment. That is, the headset can produce interactive features that the user can manipulate to adjust the tool's positioning. For example, the interactive elements can include touch-sensitive areas that allow the surgeon to make fine adjustments. The smart headset can be configured to receive an instruction from an individual operating the smart headset via an input device of the smart headset. That is, the headset can accept commands from the user through different input methods. For example, the input device can include buttons, touchscreens, or voice commands. The smart headset can be configured to lock the virtual tool superimposed within the environment, such that the virtual tool remains stationary at the desired location and the desired three-dimensional insertion angle as the smart headset changes positions within the environment. That is, the virtual tool can stay fixed in place even if the user moves the headset. For example, the tool's virtual position can remain unchanged while the user looks around. The instruction from the individual can be at least one of an eye movement, a gesture, an auditory pattern, a movement pattern, haptic feedback, a biometric input, intangible feedback, or a preconfigured interaction. That is, the user can provide instructions through various means such as gestures or voice patterns. For example, a hand gesture can lock the tool in place.

In some implementations, the visual indicia can include concentric circles indicating thresholds of the desired three-dimensional insertion angle of the surgical tool. That is, the visual elements can display concentric circles to show acceptable ranges for the insertion angle. For example, the circles can guide the user to stay within a safe angular margin. The concentric circles can include a first set of concentric circles indicating the orientation of the surgical tool at the desired location based on the desired three-dimensional insertion angle and a second set of concentric circles indicating a live orientation of the surgical tool. That is, one set of circles can show the target orientation while another set shows the real-time position. For example, this dual display can help the user correct any deviations from the planned path. The smart headset can be calibrated to the surgical tool based on the physical elements or fiducial markers (or geometric shape) to indicate the live orientation of the surgical tool. That is, the headset can use physical markers on the tool to continually track its orientation. For example, calibration can involve setting up reference points on the tool that the headset recognizes. The environmental data can include orientation data of the surgical tool, and the smart headset can be configured to continually collect the environmental data in real-time. That is, the headset can gather ongoing data about the tool's orientation. For example, this data collection can occur every few milliseconds to ensure accuracy. In response to continually collecting the environmental data, the smart headset can be configured to automatically update the at least one graphical element superimposed within the environment in real-time. That is, the visual elements can adjust dynamically based on the new data. For example, if the tool moves, the displayed path can update to reflect the new position.

In some implementations, the smart headset can include a gyroscope, and generating and displaying the at least one graphical element can be based on continually collecting orientation data of the smart headset in real-time by the gyroscope. That is, the gyroscope can provide continuous orientation data to help stabilize the visual elements. For example, the gyroscope can detect head movements and adjust the display accordingly. In response to continually collecting the orientation data of the smart headset, the smart headset can be configured to automatically update the at least one graphical element superimposed within the environment in real-time. That is, the headset can use the gyroscope data to keep the visual elements aligned with the user's view. For example, as the user looks around, the graphical elements can move in sync with their head movements. The smart headset can be configured to capture additional environmental data of the environment via an input device of the smart headset. That is, the headset can gather more information about the surroundings to enhance the accuracy of the visual aids. For example, capturing room dimensions can help in precisely overlaying the graphical elements. In some implementations, the input device can be at least one of a camera, sensor, or internet of things (IoT) device. That is, various devices can be used to collect the data. For example, a camera can capture images of the operating room, while sensors can detect physical parameters. The additional environmental data can include orientation data of a portion of a body, indicating at least one of an axial plane, coronal plane, or a sagittal plane associated with the anatomy of the portion of the body. That is, the data can help determine the orientation of the patient's body. For example, knowing the sagittal plane can assist in aligning the surgical tool with the patient's anatomy.

In some implementations, the smart headset can be configured to determine the orientation of the portion of the body within the environment based on inputting the orientation data into a machine learning algorithm and receiving an output prediction indicating the orientation of the portion of the body within the environment. That is, the headset can use machine learning to analyze the body orientation data and predict the positioning. For example, the algorithm can identify the correct alignment based on patterns in the data. Generating the at least one graphical element including the visual indicia for orienting the surgical tool at the desired location can be further based on the orientation of the portion of the body within the environment. That is, the body's orientation can influence how the visual guides can be created. For example, the graphical elements can adapt to match the patient's posture. The smart headset can be configured to generate visual indicator elements indicating the orientation of the portion of the body within the environment and display the visual indicator elements superimposed within the environment. That is, the headset can create markers that show the patient's body orientation. For example, these markers can help the surgeon align the tool with anatomical landmarks.

In some implementations, the surgical tool can be one of a gear shift probe, a pedicle probe, a Jamshidi needle, an awl, a tap, a screw inserter, a drill, or a syringe, and the environmental data can include planning data for performing an operation at the desired location using the surgical tool. That is, the method can support different types of surgical tools used for various procedures. For example, each tool type can have associated planning data to guide its use. The smart headset can be configured to receive and store diagnostic images of a portion of a body. That is, the headset can handle images that aid in the surgical process. For example, storing diagnostic images can provide reference visuals for the user. In some implementations, generating the at least one graphical element can be further based on the diagnostic images of the portion of the body. That is, the diagnostic images can enhance the accuracy of the visual aids. For example, they can help in creating overlays that match the patient's anatomy.

Generally, the processing circuits of method 4100 can be configured to perform a method for orienting a surgical tool at a desired three-dimensional insertion angle at a desired location within an environment for use in installing a medical device by using and displaying at least one graphical element. That is, the method can involve configuring processing circuits to manage the orientation of surgical tools within a three-dimensional space. For example, the processing circuits can control the alignment of the tool relative to a target in the operating environment. The processing circuits can be configured to initiate a smart headset to be calibrated to the environment so that the smart headset knows its position relative to the environment when the smart headset moves in the environment. That is, the processing circuits can start the calibration process of the smart headset to map its spatial coordinates. For example, the headset can use reference points in the room to establish its position. The processing circuits can be configured to collect, by the smart headset, environmental data of the surgical tool within the environment using physical elements or fiducial markers of the surgical tool that can be located at the desired location. That is, the circuits can gather data about the tool's physical properties and position within the environment. For example, sensors on the tool can transmit location data to the headset. The processing circuits can be configured to calculate, by the smart headset, an orientation of the surgical tool based on collecting the physical elements or fiducial markers of the surgical tool. That is, the circuits can determine the tool's orientation using the collected data. For example, the system can analyze the angle and direction of the tool. The processing circuits can be configured to receive, by the smart headset, the desired three-dimensional insertion angle and determine the position of the desired three-dimensional insertion angle at the desired location. That is, the headset can receive input for the desired insertion angle and calculate its position within the space. For example, the angle can guide the tool's insertion path. The processing circuits can be configured to generate, by the smart headset, at least one graphical element including visual indicia for orienting the surgical tool at the desired three-dimensional insertion angle at the desired location. That is, the circuits can create visual aids that help in positioning the tool accurately. For example, graphical overlays can show the intended path of the tool. The processing circuits can be configured to display, by the smart headset, the at least one graphical element superimposed within the environment. That is, the headset can project these visual elements onto the user's view. For example, augmented reality can help visualize the tool's trajectory.

In some implementations, the visual indicia can include a virtual tool for orienting the surgical tool at the desired location and the desired three-dimensional insertion angle. That is, the visual elements can display a virtual representation of the tool to guide its orientation. For example, the virtual tool can help the surgeon align the real tool accurately. The visual indicia can further include a three-dimensional vector including a guideline indicating a trajectory of the virtual tool. That is, the visual aids can also include a trajectory line showing the tool's path. For example, the vector can assist in maintaining the correct insertion angle. The processing circuits can be configured to generate, by the smart headset, interactive elements for interacting with the smart headset. That is, the circuits can create interactive features that the user can manipulate. For example, touch-sensitive controls can allow the surgeon to adjust the tool's position. The processing circuits can be configured to display, by the smart headset, the interactive elements superimposed within the environment. That is, the headset can show these interactive features within the user's field of view. For example, virtual buttons can appear on the headset's display.

In some implementations, the processing circuits can be configured to receive, by an input device of the smart headset, an instruction from an individual operating the smart headset. That is, the circuits can accept commands from the user through various input methods. For example, voice recognition can allow the user to control the tool hands-free. The processing circuits can be configured to lock, by the smart headset, the virtual tool superimposed within the environment. That is, the virtual tool can remain fixed in position despite movements of the headset. For example, once locked, the virtual tool does not move even if the user changes their viewpoint. In some implementations, the virtual tool can be stationary at the desired location and the desired three-dimensional insertion angle as the smart headset changes positions within the environment. That is, the virtual tool can stay at the set angle and location regardless of headset movements. For example, the user can walk around the room while the tool's virtual representation remains fixed. The instruction from the individual can be at least one of an eye movement, a gesture, an auditory pattern, a movement pattern, haptic feedback, a biometric input, intangible feedback, or a preconfigured interaction. That is, the user can control the tool using various input methods. For example, an eye movement can signal the system to lock the tool's position.

In some implementations, the visual indicia can include concentric circles indicating thresholds of the desired three-dimensional insertion angle of the surgical tool. That is, the visual aids can show concentric circles to indicate acceptable insertion angles. For example, the circles can guide the user to maintain the tool within an angular range. The concentric circles can include a first set of concentric circles indicating the orientation of the surgical tool at the desired location based on the desired three-dimensional insertion angle and a second set of concentric circles indicating a live orientation of the surgical tool. That is, one set of circles can show the target orientation while another set displays the current tool orientation. For example, this can help the user correct any deviations during the procedure. The smart headset can be calibrated to the surgical tool based on the physical elements or fiducial markers (or geometric shape) to indicate the live orientation of the surgical tool. That is, the headset can use the tool's physical features to track its live orientation. For example, sensors on the tool can continually relay its position to the headset.

In some implementations, the environmental data can include orientation data of the surgical tool, and the processing circuits can be configured to continually collect the environmental data in real-time. That is, the circuits can gather ongoing data about the tool's orientation. For example, real-time data collection can verify the tool remains accurately positioned. In response to continually collecting the environmental data, the processing circuits can be configured to automatically update, by the smart headset in real-time, the at least one graphical element superimposed within the environment. That is, the visual elements can adjust dynamically based on new data. For example, if the tool moves, the graphical elements can shift to reflect the new position.

In some implementations, the smart headset can include a gyroscope, and generating and displaying the at least one graphical element can be based on continually collecting, by the gyroscope in real-time, orientation data of the smart headset. That is, the gyroscope can provide continuous data to help stabilize the visual elements. For example, it can detect head movements and adjust the display accordingly. In response to continually collecting the orientation data of the smart headset, the processing circuits can be configured to automatically update, by the smart headset in real-time, the at least one graphical element superimposed within the environment. That is, the headset can use gyroscope data to keep the visual elements aligned. For example, as the user looks around, the graphical elements can move in sync with their head movements.

In some implementations, the processing circuits can be configured to capture, by an input device of the smart headset, additional environmental data of the environment. That is, the circuits can gather more information about the surroundings to enhance the accuracy of the visual aids. For example, capturing room dimensions can help in precisely overlaying the graphical elements. In some implementations, the input device can be at least one of a camera, sensor, or internet of things (IoT) device. That is, various devices can be used to collect the data. For example, a camera can capture images of the operating room, while sensors can detect physical parameters. The additional environmental data can include orientation data of a portion of a body, indicating at least one of an axial plane, coronal plane, or a sagittal plane associated with the anatomy of the portion of the body. That is, the data can help determine the orientation of the patient's body. For example, knowing the sagittal plane can assist in aligning the surgical tool with the patient's anatomy.

In some implementations, the processing circuits can be configured to determine the orientation of the portion of the body within the environment based on inputting the orientation data into a machine learning algorithm and receiving an output prediction indicating the orientation of the portion of the body within the environment. That is, the circuits can use machine learning to analyze the body orientation data and predict the positioning. For example, the algorithm can identify the correct alignment based on patterns in the data. Generating the at least one graphical element including the visual indicia for orienting the surgical tool at the desired location can be further based on the orientation of the portion of the body within the environment. That is, the body's orientation can influence how the visual guides can be created. For example, the graphical elements can adapt to match the patient's posture. The processing circuits can be configured to generate visual indicator elements indicating the orientation of the portion of the body within the environment and display the visual indicator elements superimposed within the environment. That is, the circuits can create markers that show the patient's body orientation. For example, these markers can help the surgeon align the tool with anatomical landmarks.

In some implementations, the surgical tool can be one of a gear shift probe, a pedicle probe, a Jamshidi needle, an awl, a tap, a screw inserter, a drill, or a syringe, and the environmental data can include planning data for performing an operation at the desired location using the surgical tool. That is, the method can support different types of surgical tools used for various procedures. For example, each tool type can have associated planning data to guide its use. The processing circuits can be configured to receive and store diagnostic images of a portion of a body. That is, the circuits can manage images that aid in the surgical process. For example, storing diagnostic images can provide reference visuals for the user. In some implementations, generating the at least one graphical element can be further based on the diagnostic images of the portion of the body. That is, the diagnostic images can enhance the accuracy of the visual aids. For example, they can help in creating overlays that match the patient's anatomy.

In some implementations, a method can be implemented that can orient a surgical tool at a desired three-dimensional insertion angle at a desired location within an environment for use in installing a medical device using and displaying at least one graphical element. The method can be configured to initiate a smart headset to be calibrated to the environment so that the smart headset knows its position relative to the environment when the smart headset moves in the environment. That is, the method can involve setting up the smart headset to recognize its spatial coordinates in the environment accurately. For example, the calibration process can utilize known reference points within the operating room. The method can be configured to collect, by the smart headset, environmental data of the surgical tool within the environment. That is, the smart headset can gather data regarding the tool's position and orientation. For example, sensors on the tool can transmit real-time data to the headset. In some implementations, the environmental data includes at least one of a gravitational vector and a two-dimensional plane relative to a portion of a body. That is, the data can provide information on gravitational pull and spatial orientation relative to the patient's body. For example, this data can help in maintaining the tool's alignment during procedures. The method can be configured to calculate, by the smart headset, an orientation of the surgical tool based on collecting the physical elements or fiducial markers of the surgical tool. That is, the smart headset can determine the tool's exact positioning and angle. For example, calculations can be based on the real-time data collected from the tool's sensors. The smart headset can be configured to receive the desired three-dimensional insertion angle and determine the position of the desired three-dimensional insertion angle at the desired location. That is, the headset can take the required insertion angle and translate it into a spatial coordinate within the environment. For example, this can guide the tool's path during insertion. The smart headset can be configured to generate the at least one graphical element including visual indicia for orienting the surgical tool at the desired three-dimensional insertion angle at the desired location. That is, the headset can create visual cues that help in positioning the tool accurately. For example, graphical overlays can include arrows or lines showing the optimal insertion path. The smart headset can be configured to display the at least one graphical element superimposed within the environment. That is, the headset can overlay these visual elements onto the user's view of the environment. For example, the surgeon can see the graphical indicators directly on the display.

In some implementations, a system for orienting a tool at a desired location within an environment can include an electronic device and a smart headset including a transparent display and communicatively coupled to the electronic device. The smart headset can be configured to initiate a smart headset to be calibrated to the environment so that the smart headset knows its position relative to the environment when the smart headset moves in the environment. That is, the system can involve calibrating the smart headset to understand its spatial location within the environment. For example, calibration can use reference markers within the operating room. The smart headset can be configured to receive, from the electronic device communicatively coupled to the smart headset, environmental data indicating the position of the surgical tool within the environment. That is, the headset can collect data from the electronic device about the tool's position. For example, the data can include coordinates and orientation. The smart headset can be configured to receive, from the electronic device, the desired three-dimensional insertion angle. That is, the headset can obtain the insertion angle information from the electronic device. For example, the angle data can help guide the surgical tool. The smart headset can be configured to generate at least one graphical element including visual indicia for orienting the surgical tool at the desired three-dimensional insertion angle at the desired location. That is, the headset can create visual elements to aid in tool positioning. For example, graphical elements can display the path and orientation of the tool. The smart headset can be configured to display the at least one graphical element superimposed within the environment. That is, the headset can project these visual aids onto the user's view. For example, augmented reality can help the user see the insertion path directly on the headset's display.

In some implementations, a system for orienting a tool at a desired location within an environment can include a smart headset including a transparent display and a processing circuit communicatively coupled to the smart headset. The processing circuits can be configured to determine a desired three-dimensional insertion angle of the surgical tool based on the orientation of the surgical tool. That is, the processing circuits can analyze the tool's current position to calculate the insertion angle. For example, data from the tool's sensors can be used to determine the correct angle. The processing circuits can be configured to collect environmental data of the surgical tool within the environment. That is, the circuits can gather data on the tool's spatial coordinates. For example, environmental data can include the tool's location and orientation within the room. The processing circuits can be configured to generate at least one graphical element including visual indicia for orienting the surgical tool at the desired location based on the three-dimensional insertion angle. That is, the circuits can create visual guides to help align the tool correctly. For example, graphical elements can display the insertion path and alignment markers. The processing circuits can be configured to display, on a smart headset communicatively coupled to the one or more processors, the at least one graphical element superimposed within the environment. That is, the headset can show these visual aids in the user's field of view. For example, the display can overlay the graphical elements onto the real-world environment.

In some implementations, a smart headset for orienting a tool at a desired location within an environment can include a transparent display, a plurality of sensor devices, and one or more processors. The one or more processors can be configured to initiate the smart headset to be calibrated to the environment so that the smart headset knows its position relative to the environment when the smart headset moves in the environment. That is, the headset's processors can set up the device to recognize its spatial location accurately. For example, calibration can involve mapping the room's layout and reference points. The one or more processors can be configured to collect, via the plurality of sensor devices, environmental data of the surgical tool within the environment using physical elements or fiducial markers of the surgical tool that can be located at the desired location. That is, the processors can use sensors to gather data about the tool's position and physical characteristics. For example, the tool can have markers that provide positional information. The one or more processors can be configured to calculate an orientation of the surgical tool based on collecting the physical elements or fiducial markers of the surgical tool. That is, the processors can determine the tool's alignment and angle using the collected data. For example, the sensors can relay real-time orientation data to the headset. The one or more processors can be configured to receive a desired three-dimensional insertion angle. That is, the processors can obtain the insertion angle required for the procedure. For example, this angle can guide the tool's insertion path. The one or more processors can be configured to determine the position of the desired three-dimensional insertion angle at the desired location. That is, the processors can translate the insertion angle into a spatial coordinate. For example, the tool's path can be adjusted based on this angle. The one or more processors can be configured to generate at least one graphical element including visual indicia for orienting the surgical tool at the desired three-dimensional insertion angle at the desired location. That is, the processors can create visual guides to assist in positioning the tool. For example, graphical elements can show the optimal insertion path and angle. The one or more processors can be configured to display, via the transparent display, the at least one graphical element superimposed within the environment. That is, the headset can project these visual aids onto the user's view. For example, augmented reality can overlay the graphical elements onto the user's field of vision.

In some implementations, a smart headset for orienting a tool at a desired location within an environment can include an opaque display, a plurality of sensor devices, and one or more processors. The one or more processors can be configured to initiate the smart headset to be calibrated to the environment so that the smart headset knows its position relative to the environment when the smart headset moves in the environment. That is, the processors can set up the headset to recognize its spatial coordinates accurately. For example, calibration can involve using reference markers within the operating room. The one or more processors can be configured to collect, via the plurality of sensor devices, environmental data within the environment. That is, the processors can gather information about the surroundings to enhance accuracy. For example, sensors can detect physical parameters like distance and orientation. The one or more processors can be configured to calculate an orientation of the surgical tool based on the collected environmental data within the environment. That is, the processors can determine the tool's angle and alignment using the data collected. For example, orientation data can be analyzed to adjust the tool's positioning. The one or more processors can be configured to receive a desired three-dimensional insertion angle. That is, the processors can obtain the required insertion angle for the procedure. For example, this angle can help guide the tool's path. The one or more processors can be configured to determine the position of the desired three-dimensional insertion angle at the desired location. That is, the processors can convert the insertion angle into a spatial coordinate. For example, the tool's path can be calculated based on this angle. The one or more processors can be configured to generate at least one graphical element including visual indicia for orienting the surgical tool at the desired three-dimensional insertion angle at the desired location. That is, the processors can create visual guides to assist in tool positioning. For example, graphical elements can show the tool's path and orientation. The one or more processors can be configured to display, via the opaque display, the at least one graphical element superimposed within the environment. That is, the headset can project these visual aids onto the user's view. For example, augmented reality can overlay the graphical elements onto the user's field of vision.

In some implementations, the environmental data can include one or more of positional data of the environment, body features of a user of the smart headset, and physical elements or fiducial markers (or geometric shape) of the surgical tool. That is, the data can provide information about the environment and the tool. For example, positional data can help in mapping the tool's exact location, while body features can assist in understanding the user's interaction with the tool. The surgical tool can be one of a pedicle screw, an interbody cage, a stent, a pin, a rod, or a graft, and the environmental data can include planning data for inserting the surgical tool. That is, the system can support various surgical tools used for different procedures. For example, each tool can have planning data that guides its insertion process.

FIG. 39 illustrates an example flowchart of a method 3900 for orienting a surgical tool at a desired three-dimensional insertion angle at a desired location within an environment, according to example implementations. Smart headset 2940 can be configured to perform method 3900. Further, any computing device described herein can be configured to perform method 3900.

In broad overview of method 3900, at block 3910, the smart headset (e.g., smart headset 2940 of FIGS. 1 and 42) can initiate a smart headset. At block 3920, the smart headset can receive environmental data. At block 3930, the smart headset can receive the desired three-dimensional insertion angle. At block 3940, the smart headset can generate at least one graphical element. At block 3950, the smart headset can display the at least one graphical element. Additional, fewer, or different operations may be performed depending on the particular arrangement. In some arrangements, some, or all operations of method 3900 may be performed by one or more processors executing on one or more computing devices, systems, or servers. In various arrangements, each operation may be re-ordered, added, removed, or repeated.

Referring to method 3900 in more detail, at block 3910, the system can initiate a smart headset. At block 3920, the system may receive environmental data. At block 3930, the system may receive the desired three-dimensional insertion angle. At block 3940, the system may generate at least one graphical element. At block 3950, the system may display the at least one graphical element.

FIG. 40 illustrates another example flowchart of a method 4000 for orienting a surgical tool at a desired three-dimensional insertion angle at a desired location within an environment, according to example implementations. Electronic device 2920 can be configured to perform method 4000. Further, any computing device described herein can be configured to perform method 4000.

In broad overview of method 4000, at block 4010, the processing circuit (e.g., electronic device 2920 of FIG. 1) can determine a desired three-dimensional angle. At block 4020, the processing circuit can collect environmental data. At block 4030, the processing circuit can generate at least one graphical element. At block 4040, the processing circuit can display at least one graphical element. Additional, fewer, or different operations may be performed depending on the particular arrangement. In some arrangements, some, or all operations of method 4000 may be performed by one or more processors executing on one or more computing devices, systems, or servers. In various arrangements, each operation may be re-ordered, added, removed, or repeated.

Referring to method 4000 in more detail, at block 4010, the system may determine a desired three-dimensional angle. At block 4020, the system may collect environmental data. At block 4030, the system may collect environmental data. At block 4030, the system may generate at least one graphical element. At block 4040, the system may display the at least one graphical element.

FIG. 41 illustrates another example flowchart of a method for orienting a surgical tool at a desired three-dimensional insertion angle at a desired location within an environment, according to example implementations. Smart headset 2940 can be configured to perform method 4100. Further, any computing device described herein can be configured to perform method 4100.

In broad overview of method 4100, at block 4110, the smart headset (e.g., smart headset 2940 of FIGS. 1 and 42) can initiate a smart headset. At block 4120, the smart headset can collect environmental data. At block 4130, the smart headset can calculate an orientation of the surgical tool. At block 4140, the smart headset can receive the desired three-dimensional insertion angle. At block 4150, the smart headset can determine the position of the desired three-dimensional insertion angle. At block 4160, the smart headset can generate at least one graphical element. At block 4170, the smart headset can display the at least one graphical element. Additional, fewer, or different operations may be performed depending on the particular arrangement. In some arrangements, some, or all operations of method 4100 may be performed by one or more processors executing on one or more computing devices, systems, or servers. In various arrangements, each operation may be re-ordered, added, removed, or repeated.

Referring to method 4100 in more detail, at block 4110, the system may initiate a smart headset. At block 4120, the system may collect environmental data. At block 4130, the system may calculate an orientation of the surgical tool. At block 4140, the system may receive the desired three-dimensional insertion angle. At block 4150, the system may determine the position of the desired three-dimensional insertion angle. At block 4160, the system may generate at least one graphical element. At block 4170, the system may display the at least one graphical element.

Referring now to FIG. 42, a block diagram of the smart headset 2940 is shown, according to some implementations. The smart headset 2940 includes a network interface 4222, a processing circuit 4224, and an input/output device 4240. The network interface 4222 can be structured and used to establish connections with other computing systems and devices (e.g., electronic devices 2920 or electronic device 2960) via the network 2902 (or 2904, 2905, 2906, and 2908). The network interface 4222 includes program logic that facilitates connection of the smart headset 2940 to the network 2902. For example, the network interface 4222 may include any combination of a wireless network transceiver (e.g., a cellular modem, a Bluetooth transceiver, a Wi-Fi transceiver, etc.) and/or a wired network transceiver (e.g., an Ethernet transceiver). In some arrangements, the network interface 4222 includes the hardware (e.g., processor, memory, and so on) and machine-readable media sufficient to support communication over multiple channels of data communication. Further, in some arrangements, the network interface 4222 includes cryptography capabilities to establish a secure or relatively secure communication session in which data communicated over the session can be encrypted.

The processing circuit 4224 includes a processor(s) 4226, a memory 4228, and an input/output device 4240. The memory 4228 may be one or more devices (e.g., RAM, ROM, Flash memory, hard disk storage) for storing data and/or computer code for completing and/or facilitating the various processes described herein. The memory 4228 may be or include non-transient volatile memory, non-volatile memory, and non-transitory computer storage media. Memory 4228 may include database components, object code components, script components, or any other type of information structure for supporting the various activities and information structures described herein. Memory 4228 may be communicably coupled to the processor(s) 4226 and include computer code or instructions for executing one or more processes described herein. The processor 114 may be implemented as one or more application specific integrated circuits (ASICs), field programmable gate arrays (FPGAs), a group of processing components, or other suitable electronic processing components. As such, the smart headset 2940 can be configured to run a variety of application programs and store associated data in a database of the memory 116 (e.g., smart headset database 4229). One such application may be the smart headset client application 4238. The memory 4228 may store a smart headset database 4229, according to some implementations. The smart headset database 4229 may be configured to store various data used in installing a medical device (e.g., graphical elements, environmental data, calibration data, orientation data, human anatomy data, etc.)

In some implementations, the smart headset client application 4238 may be incorporated with an existing application in use by the smart headset 2940 (e.g., a mobile provider application, a service provider application, provided by electronic device 2920 or 2960). In other implementations, the smart headset client application 4238 can be a separate software application implemented on the smart headset 2940. The smart headset client application 4238 may be downloaded by the smart headset 2940 prior to its usage, hard coded into the memory 4224 of the smart headset 2940 or be a network-based or web-based interface application such that the smart headset 2940 may provide a web browser or via network communication (e.g., 2902) to access the application, which may be executed remotely from the smart headset 2940. Accordingly, the smart headset client application 4238 may include software and/or hardware capable of implementing a network-based or web-based application. For example, in some instances, the smart headset client application 4238 includes software such as HTML, XML, WML, SGML, PUP (Hypertext Preprocessor), CGI, and like languages.

In the latter instance, a user (e.g., a doctor) may log onto or access the web-based interface before usage of the application (e.g., before surgery). In this regard, the smart headset client application 4238 may be supported by a separate computing system (e.g., electronic device 2920 or 2960) including one or more servers, processors, network interface (sometimes referred to herein as a “network circuit”), and so on, that transmit applications for use to the smart headset 2940. In certain implementations, the smart headset client application 4238 includes an application programming interface (API) and/or a software development kit (SDK) that facilitate the integration of other applications with the smart headset client application 4238. For example, the smart headset client application 4238 can be configured to utilize the functionality of the electronic devices 2920 and 2960 by interacting with the devices through an API. In some implementations, the smart headset client application 4238 can be configured to communicate with the electronic devices (e.g., 2920 and 2960). Accordingly, the smart headset 2940 can be communicably coupled to the electronic devices (e.g., 2920 and 2960), via various networks (e.g., network 2902, 2904, 2905, 2906, and 2908).

The smart headset client application 4238 may therefore communicate with the electronic devices 2920 and 2960, to perform several functions. For example, the smart headset client application 4238 can be configured to receive data from the electronic devices 2920 and/or 2960 pertaining to visual indicia and/or graphical elements for orientating the surgical tool 2910 (sometimes referred to herein as a “surgical device”). In this example, the smart headset client application 4238 may magnify, highlight, color, bold, and/or variously emphasize orientations of the surgical tool 2910. In another example, the smart headset client application 4238 can be configured to receive data from the electronic device 2920 or 2960 and overlay concentric circles within display 3301. In this example, the smart headset client application 4238 may provide notifications, tools (e.g., settings icons, lock options, latitudes and longitudes of the surgical tool 2910, concentric circles, and so on).

Still referring to FIG. 42, the input/output device 4240 can be structured to receive communications from and provide communications to the electronic devices 2920 and 2960. The input/output device 4240 can be structured to exchange data, communications, instructions, etc. with an input/output component of the electronic devices 2920 and 2960. In some implementations, the input/output device 4240 includes communication circuitry for facilitating the exchange of data, values, messages, and the like between the input/output device 4240 and the components of the electronic devices 2920 and 2960. In yet another embodiment, the input/output device 4240 includes machine-readable media for facilitating the exchange of information between the input/output device and the components of the electronic devices 2920 and 2960. In yet another embodiment, the input/output device 4240 includes any combination of hardware components, communication circuitry, and machine-readable media.

In some implementations, the input/output device 4240 includes suitable input/output ports and/or uses an interconnect bus (not shown) for interconnection with a local display (e.g., a touchscreen display) and/or keyboard/mouse devices (when applicable), or the like, serving as a local user interface for programming and/or data entry, retrieval, or other user interaction purposes. As such, the input/output device 4240 may provide an interface for the user to interact with various applications (e.g., the smart headset client application 4238). For example, the input/output device 4240 includes a camera, a speaker, a touch screen, a microphone, a biometric device, other IoT Devices, a virtual reality headset display, a smart glasses display, and the like. As used herein, virtual reality, augmented reality, and mixed reality may each be used interchangeably yet refer to any kind of extended reality, including virtual reality, augmented reality, and mixed reality.

In some implementations, the input/output device 4240 of the smart headset 2940 can be similarly structured to receive communications from and provide communications to the electronic devices (e.g., 2920 and 2960) paired (e.g., via a network connection, communicably coupled, via Bluetooth, via a shared connection, and so on) with a smart headset 2940. In various implementations, the input/output device 4240 can include various cameras and/or sensors within the housing of the smart headset 2940. For example, the smart headset 2940 can include one or more cameras (e.g., for detecting movement, motion, and view environment), audio sensor, temperature sensor, haptic feedback sensor, biometric sensor, pulse oximetry (detect oxygen saturation of blood), altitude sensor, humidity sensor, magnetometer, accelerometer, gyroscope, stress sensors, various IoT devices 190, and so on.

In some implementations, the session management circuit 4230 can be further configured to receive sensor data from the input/output device 4240 of the smart headset 2940. For example, the session management circuit 4230 may be configured to receive camera data (e.g., environmental data) associated with surgical tool arrangement (e.g., orientation) within environment 2900, movement data from a motion detector, temperature sensor data, audio data indicating a selection and/or action, haptic feedback indicating selection action, and so on. Additionally, the session management circuit 4230 may determine when to send reminders to the display 3301. In some implementations, the session management circuit 4230 can further be configured to generate content for display to users (e.g., doctor, user, and so on). The content can be selected from among various resources (e.g., webpages, applications, databases, and so on). The session management circuit 4230 can be also structured to provide content (e.g., graphical user interface (GUI)) to the display 3301 of smart headsets 2940, for display within the resources. In various implementations, the content from which the session management circuit 4230 selects may be provided by the electronic devices 2920 and 2960 (e.g., via the networks). In some implementations, session management circuit 4230 may select content to be displayed on the smart headset 2940. In various implementations, the session management circuit 4230 may determine content to be generated and published in one or more content interfaces of resources (e.g., webpages, applications, and so on).

In various implementations, the session management circuit 4230 can include a monitoring circuit 4254. The monitoring circuit 4254 can be configured to cause the smart headset 2940 to identify a plurality of coordinate values of the graphical user interface based on relative position (e.g., vectors and planes) of items (e.g., surgical tool) within the environment 2900. In some implementations, the monitoring circuit 4254 can be configured to cause the smart headset 2940 to determine coordinates of the surgical tool relative to a reference point (or plane) within environmental 2900. In implementations, the monitoring circuit 4254 can cause the smart headset 2940 to determine a three-dimensional coordinate value of surgical tool 2910 along an x (e.g., x-axis coordinate), y (e.g., y-axis coordinate), and/or z axis (e.g., z-axis coordinate). Additional details regarding determining the orientation of the surgical tool 2910 can be described above in detail with reference to FIGS. 3-21.

In some implementations, the monitoring circuit 4254 can be configured to cause the display of the smart headset 2940 to detect if activity occurred (e.g., movement of the smart headset 2940 by the user 2950, movement of the surgical tool 2910 based on movement of the electronic device 2920 or 2960, etc.) within and/or in the environment 2900 (e.g., from an input/output device 4240). In another instance, the monitoring circuit 4254 can be configured to receive sensor input from one or more input/output device 4240 around the environment (e.g., within the space, within the building, and so on). In one example, the sensor input may be a hand gesture (e.g., wave, swipe, point) of an individual (e.g., 2950) that does not contact the touchscreen display. In one example, the sensor input may be an audible and/or visual output of an individual indicating a specific action to be performed (e.g., lock the virtual surgical tool) from one or more input/output device 4240 around the environment.

The notification generation circuit 4234 may be configured to create alerts regarding orienting a surgical tool 2910 (or medical device) at a desired three-dimensional insertion angle at a desired location, initiating a smart headset 2940, visual indicia, graphical elements, and so on. The notification generation circuit 4234 may also receive instructions on the format of a notification from the electronic device 2920 (or 2960). In some implementations, the notification generation circuit 4234 can be configured to instruct the smart headset 2940 or electronic device 2920 to provide audible and/or visual outputs to a user (e.g., doctor) regarding information displayed during an augmented reality (AR) session (e.g., a procedure upon initiating the smart headset 2940). For example, the notification generation circuit 4234 may be configured to cause visual indicia to display on display 3301. As another example, the notification generation circuit 4234 may be configured to generate multiple concentric circles (e.g., 3309A and 3309B) indicates orientation of a surgical tool 2910. It should be understood that all visual indicia and graphical elements displayed on display 3301 can be generated by notification generation circuit 4234.

Additionally, it should be understood the electronic devices 2920 and 2960 can include the same or similar circuits and applications described with reference to smart headset 2940. For example, electronic devices 2920 and 2960 can include a network interface, processing circuit, processor, memory, electronic database, session management circuit, viewport monitoring circuit, notification generation circuit, smart headset client application, and input/output device. As such, the electronic devices 2920 and 2960 can execute all tasks and actions the smart headset 2940 can execute, but instead can provide the content to the display 3301 of the smart headset 2940. In particular, the electronic devices 2920 and 2960 can be communicable coupled to the smart headset 2940, and each device/headset can execute various tasks and actions concurrently and/or sequentially.

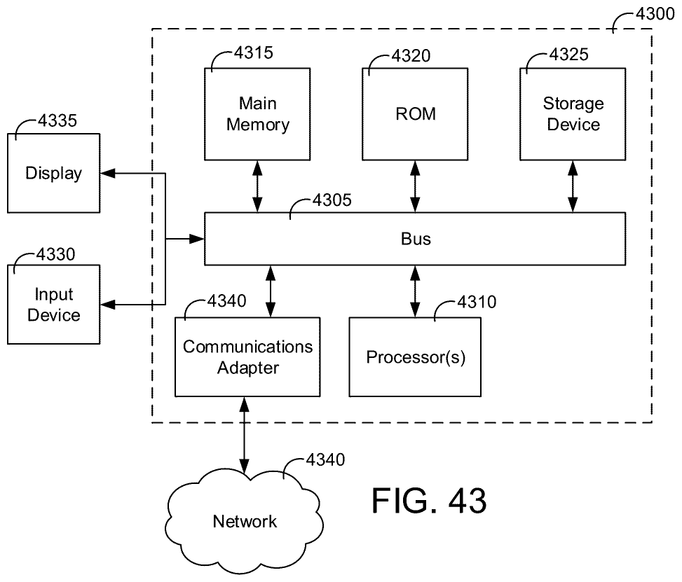

Referring now to FIG. 43, a depiction of a computer system 4300 is shown. The computer system 4300 that can be used, for example, to implement an apparatus 300, augmented reality or virtual reality based system 706, electronic device 2920, smart headset 2940, electronic device 2960, and/or various other example systems described in the present disclosure. The computing system 4300 includes a bus 4305 or other communication component for communicating information and a processor(s) 4310, which may be one or more processors, coupled to the bus 4305 for processing information. The computing system 4300 also includes main memory 4315, such as a random-access memory (RAM) or other dynamic storage device, coupled to the bus 4305 for storing information, and instructions to be executed by the processor(s) 4310. Main memory 4315 can also be used for storing position information, temporary variables, or other intermediate information during execution of instructions by the processor(s) 4310. The computing system 4300 may further include a read only memory (ROM) 4320 or other static storage device coupled to the bus 4305 for storing static information and instructions for the processor(s) 4310. A storage device 4325, such as a solid-state device, magnetic disk, or optical disk, can be coupled to the bus 4305 for persistently storing information and instructions.

The computing system 4300 may be coupled via the bus 4305 to a display 4335, such as a liquid crystal display, or active matrix display, for displaying information to a user. An input device 4330, such as a keyboard including alphanumeric and other keys, may be coupled to the bus 4305 for communicating information, and command selections to the processor(s) 4310. In another arrangement, the input device 4330 has a touch screen display 4335. The input device 4330 can include any type of biometric sensor, a cursor control, such as a mouse, a trackball, or cursor direction keys, for communicating direction information and command selections to the processors 4310 and for controlling cursor movement on the display 4335.

In some arrangements, the computing system 4300 may include a communications adapter 4340, such as a networking adapter. Communications adapter 4340 may be coupled to bus 4305 and may be configured to allow communications with a computing or communications network 4340 and/or other computing systems. In various illustrative arrangements, any type of networking configuration may be achieved using communications adapter 4340, such as wired (e.g., via Ethernet), wireless (e.g., via Wi-Fi™, Bluetooth™), satellite (e.g., via GPS) pre-configured, ad-hoc, LAN, and WAN.

According to various arrangements, the processes that effectuate illustrative arrangements that are described herein can be achieved by the computing system 4300 in response to the processor(s) 4310 executing an arrangement of instructions contained in main memory 4315. Such instructions can be read into main memory 4315 from another computer-readable medium, such as the storage device 4325. Execution of the arrangement of instructions contained in main memory 4315 causes the computing system 4300 to perform the illustrative processes described herein. One or more processors in a multi-processing arrangement may also be employed to execute the instructions contained in main memory 4315. In alternative arrangements, hard-wired circuitry may be used in place of or in combination with software instructions to implement illustrative arrangements. Thus, arrangements are not limited to any specific combination of hardware circuitry and software.

That is, although an example processing system has been described in FIG. 43, arrangements of the subject matter and the functional operations described in this specification can be carried out using other types of digital electronic circuitry, or in computer software (e.g., application, blockchain, distributed ledger technology) embodied on a tangible medium, firmware, or hardware, including the structures disclosed in this specification and their structural equivalents, or in combinations of one or more of them. Arrangements of the subject matter described in this specification can be implemented as one or more computer programs, e.g., one or more subsystems of computer program instructions, encoded on one or more computer storage medium for execution by, or to control the operation of, data processing apparatus. Alternatively, or in addition, the program instructions can be encoded on an artificially generated propagated signal, e.g., a machine generated electrical, optical, or electromagnetic signal, that can be generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices). Accordingly, the computer storage medium is both tangible and non-transitory.

Although shown in the arrangements of FIG. 43 as singular, stand-alone devices, one of ordinary skill in the art will appreciate that, in some arrangements, the computing system 4300 may include virtualized systems and/or system resources. For example, in some arrangements, the computing system 4300 may be a virtual switch, virtual router, virtual host, or virtual server. In various arrangements, computing system 4300 may share physical storage, hardware, and other resources with other virtual machines. In some arrangements, virtual resources of the network 4340 may include cloud computing resources such that a virtual resource may rely on distributed processing across more than one physical processor, distributed memory, etc.

Although the preceding description has been described herein with reference to particular means, materials and implementations, it is not intended to be limited to the particulars disclosed herein; rather, it extends to all functionally equivalent structures, methods, and uses, such as are within the scope of the appended claims.

While this specification contains many specific implementation details and/or arrangement details, these should not be construed as limitations on the scope of any implementations or of what may be claimed, but rather as descriptions of features specific to particular implementations and/or arrangements of the systems and methods described herein. Certain features that are described in this specification in the context of separate implementations and/or arrangements can also be implemented and/or arranged in combination in a single implementation and/or arrangement. Conversely, various features that are described in the context of a single implementation and/or arrangement can also be implemented and arranged in multiple implementations and/or arrangements separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Additionally, features described with respect to particular headings may be utilized with respect to and/or in combination with illustrative arrangement described under other headings; headings, where provided, are included solely for the purpose of readability and should not be construed as limiting any features provided with respect to such headings.

Similarly, while operations are depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. In addition, the processes depicted in the accompanying figures do not necessarily require the particular order shown, or sequential order, to achieve desirable results.

In certain circumstances, multitasking and parallel processing may be advantageous. Moreover, the separation of various system components in the implementations and/or arrangements described above should not be understood as requiring such separation in all implementations and/or arrangements, and it should be understood that the described program components and systems can generally be integrated together in a single software product or packaged into multiple software products.

Having now described some illustrative implementations, implementations, illustrative arrangements, and arrangements it is apparent that the foregoing is illustrative and not limiting, having been presented by way of example. In particular, although many of the examples presented herein involve specific combinations of method acts or system elements, those acts, and those elements may be combined in other ways to accomplish the same objectives. Acts, elements and features discussed only in connection with one implementation and/or arrangement are not intended to be excluded from a similar role in other implementations or arrangements.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations and/or arrangements consisting of the items listed thereafter exclusively. In one arrangement, the systems and methods described herein consist of one, each combination of more than one, or all of the described elements, acts, or components.

Any references to implementations, arrangements, or elements or acts of the systems and methods herein referred to in the singular may also embrace implementations and/or arrangements including a plurality of these elements, and any references in plural to any implementation, arrangement, or element or act herein may also embrace implementations and/or arrangements including only a single element. References in the singular or plural form are not intended to limit the presently disclosed systems or methods, their components, acts, or elements to single or plural configurations. References to any act or element being based on any information, act or element may include implementations and/or arrangements where the act or element is based at least in part on any information, act, or element.

Any implementation disclosed herein may be combined with any other implementation, and references to “an implementation,” “some implementations,” “an alternate implementation,” “various implementation,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

Any arrangement disclosed herein may be combined with any other arrangement, and references to “an arrangement,” “some arrangements,” “an alternate arrangement,” “various arrangements,” “one arrangement” or the like are not necessarily mutually exclusive and are intended to indicate that a particular feature, structure, or characteristic described in connection with the arrangement may be included in at least one arrangement. Such terms as used herein are not necessarily all referring to the same arrangement. Any arrangement may be combined with any other arrangement, inclusively or exclusively, in any manner consistent with the aspects and arrangements disclosed herein.

References to “or” may be construed as inclusive so that any terms described using “or” may indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations and/or arrangements are illustrative rather than limiting of the described systems and methods. Scope of the systems and methods described herein is thus indicated by the appended claims, rather than the foregoing description, and changes that come within the meaning and range of equivalency of the claims are embraced therein.

It should be understood that no claim element herein is to be construed under the provisions of 35 U.S.C. § 112(f), unless the element is expressly recited using the phrase “means for.”

As used herein, the term “circuit” may include hardware structured to execute the functions described herein. In some implementations, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors. In some implementations, a circuit may take the form of one or more analog circuits, electronic circuits (e.g., integrated circuits (IC), discrete circuits, system on a chip (SOC) circuits), telecommunication circuits, hybrid circuits, and any other type of “circuit.” In this regard, the “circuit” may include any type of component for accomplishing or facilitating achievement of the operations described herein. For example, a circuit as described herein may include one or more transistors, logic gates (e.g., NAND, AND, NOR, OR, XOR, NOT, XNOR), resistors, multiplexers, registers, capacitors, inductors, diodes, wiring.

The term “circuit” may also include one or more processors communicatively coupled to one or more memory or memory devices. In this regard, the one or more processors may execute instructions stored in the memory or may execute instructions otherwise accessible to the one or more processors. In some implementations, the one or more processors may be embodied in various ways. The one or more processors may be constructed in a manner sufficient to perform at least the operations described herein. In some implementations, the one or more processors may be shared by multiple circuits (e.g., circuit A and circuit B may include or otherwise share the same processor which, in some example implementations, may execute instructions stored, or otherwise accessed, via different areas of memory). Alternatively or additionally, the one or more processors may be structured to perform or otherwise execute certain operations independent of one or more co-processors. In other example implementations, two or more processors may be coupled via a bus to allow independent, parallel, pipelined, or multi-threaded instruction execution. Each processor may be implemented as one or more general-purpose processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor), microprocessor. In some implementations, the one or more processors may be external to the apparatus, for example the one or more processors may be a remote processor (e.g., a cloud based processor). Alternatively or additionally, the one or more processors may be internal and/or local to the apparatus. In this regard, a given circuit or components thereof may be disposed locally (e.g., as part of a local server, a local computing system) or remotely (e.g., as part of a remote server such as a cloud based server). To that end, a “circuit” as described herein may include components that are distributed across one or more locations.

An exemplary system for implementing the overall system or portions of the implementations might include a general purpose computing devices in the form of computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some implementations, the non-volatile media may take the form of ROM, flash memory (e.g., flash memory such as NAND, 3D NAND, NOR, 3D NOR), EEPROM, MRAM, magnetic storage, hard discs, optical discs, etc. In other implementations, the volatile storage media may take the form of RAM, TRAM, ZRAM, etc. Combinations of the above are also included within the scope of machine-readable media. In this regard, machine-executable instructions include, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components), in accordance with the example implementations described herein.

It should also be noted that the term “input devices,” as described herein, may include any type of input device including, but not limited to, a keyboard, a keypad, a mouse, joystick or other input devices performing a similar function. Comparatively, the term “output device,” as described herein, may include any type of output device including, but not limited to, a computer monitor, printer, facsimile machine, or other output devices performing a similar function.

Any foregoing references to currency or funds are intended to include fiat currencies, non-fiat currencies (e.g., precious metals), and math-based currencies (often referred to as cryptocurrencies). Examples of math-based currencies include Bitcoin, Litecoin, Dogecoin, and the like.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative implementations. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps and decision steps.

Any reference to processor can utilize computing technologies such as one or more general-purpose microprocessors (uP) and/or digital signal processors (DSP) with associated storage memory such a Flash, ROM, RAM, SRAM, DRAM or other like technologies for controlling operations of the aforementioned components of the terminal device. The instructions may also reside, completely or at least partially, within other memory, and/or a processor during execution thereof by another processor or computer system, local or remote.

The electronic circuitry of the processor (or controller) can include one or more Application Specific Integrated Circuit (ASIC) chips or Field Programmable Gate Arrays (FPGAs), for example, specific to a core signal processing algorithm or control logic. The processor can be an embedded platform running one or more modules of an operating system (OS). In one arrangement, the storage memory may store one or more sets of instructions (e.g., software) embodying any one or more of the methodologies or functions described herein.

The illustrations of implementations described herein are intended to provide a general understanding of the structure of various implementations, and they are not intended to serve as a complete description of all the elements and features of apparatus and systems that might make use of the structures described herein. Many other implementations will be apparent to those of skill in the art upon reviewing the above description. Other implementations may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Although specific implementations have been illustrated and described herein, it should be appreciated that any arrangement calculated to achieve the same purpose may be substituted for the specific implementations shown. This disclosure is intended to cover any and all adaptations or variations of various implementations. Combinations of the above implementations, and other implementations not specifically described herein, will be apparent to those of skill in the art upon reviewing the above description.

Where applicable, the present implementations can be realized in hardware, software or a combination of hardware and software. Any kind of computer system or other apparatus adapted for carrying out the methods described herein are suitable. A typical combination of hardware and software can be a mobile communications device with a computer program that, when being loaded and executed, can control the mobile communications device such that it carries out the methods described herein. Portions of the present method and system may also be embedded in a computer program product, which includes all the features enabling the implementation of the methods described herein and which when loaded in a computer system, is able to carry out these methods.

While the preferred implementations have been illustrated and described, it will be clear that the implementations are not so limited. Numerous modifications, changes, variations, substitutions and equivalents will occur to those skilled in the art without departing from the spirit and scope of the present implementations as defined by the appended claims.

What is claimed is:

1. A method for orienting a surgical tool at a desired three-dimensional insertion angle at a desired location within an environment for use in installing a medical device by using and displaying at least one graphical element, the method comprising:
initiating a smart headset to be calibrated to the environment so that a position of the smart headset is known relative to the environment when the smart headset moves in the environment;
receiving, by the smart headset from an electronic device communicatively coupled to the smart headset, environmental data indicating the position of the surgical tool within the environment;
receiving, by the smart headset from the electronic device, the desired three-dimensional insertion angle;
generating, by the smart headset, at least one graphical element comprising visual indicia for orienting the surgical tool at the desired three-dimensional insertion angle at the desired location; and
displaying, by the smart headset, the at least one graphical element superimposed within the environment.

2. The method of claim 1, wherein the visual indicia comprises a virtual tool for orienting the surgical tool at the desired location and the desired three-dimensional insertion angle, and wherein the visual indicia further comprise a three-dimensional vector comprising a guideline indicating a trajectory of the virtual tool, and wherein the method further comprising:
generating, by the smart headset, interactive elements for interacting with the smart headset; and
displaying, by the smart headset, the interactive elements superimposed within the environment.

3. The method of claim 2, further comprising:
receiving, by an input device of the smart headset, an instruction from an individual operating the smart headset;
locking, by the smart headset, the virtual tool superimposed within the environment, wherein the virtual tool is stationary at the desired location and the desired three-dimensional insertion angle as the smart headset changes positions within the environment; and
wherein the instruction from the individual is at least one of an eye movement, a gesture, an auditory pattern, a movement pattern, haptic feedback, a biometric input, intangible feedback, or a preconfigured interaction.

4. The method of claim 1, wherein:
the visual indicia comprises concentric circles indicating thresholds of the desired three-dimensional insertion angle of the surgical tool;
the concentric circles comprise a first set of concentric circles at the desired location based on the desired three-dimensional insertion angle and a second set of concentric circles indicating a live orientation of the surgical tool;
the electronic device is calibrated to the surgical tool to indicate the live orientation of the surgical tool; and
the environmental data comprises orientation data of the surgical tool, and wherein the smart headset continually receives the environmental data from the electronic device in real-time.

5. The method of claim 4, further comprising:
in response to continually receiving the environmental data, automatically updating, by the smart headset in real-time, the at least one graphical element superimposed within the environment, wherein the smart headset comprises a gyroscope, and wherein generating and displaying the at least one graphical element is based on continually collecting, by the gyroscope in real-time, orientation data of the smart headset; and
in response to continually collecting the orientation data of the smart headset, automatically updating, by the smart headset in real-time, the at least one graphical element superimposed within the environment.

6. The method of claim 1, further comprising:
capturing, by an input device of the smart headset, additional environmental data of the environment, wherein the input device is at least one of a camera, sensor, or internet of things (IoT) device, wherein the additional environmental data comprises orientation data of a portion of a body, and wherein the orientation data of the portion of the body indicates at least one of an axial plane, coronal plane, or a sagittal plane associated with anatomy of the portion of the body; and
determining, by the smart headset, an orientation of the portion of the body within the environment based on inputting the orientation data into a machine learning algorithm and receiving an output prediction indicating the orientation of the portion of the body within the environment.

7. The method of claim 6, wherein generating the at least one graphical element comprising the visual indicia for orienting the surgical tool at the desired location is further based on the orientation of the portion of the body within the environment, and wherein the method further comprising:
generating, by the smart headset, visual indicator elements indicating the orientation of the portion of the body within the environment; and
displaying, by the smart headset, the visual indicator elements superimposed within the environment.

8. The method of claim 1, wherein the surgical tool is one of a gear shift probe, a pedicle probe, a Jamshidi needle, an awl, a tap, a screw inserter, a drill, or a syringe, and wherein the environmental data comprises planning data for performing an operation at the desired location using the surgical tool, and wherein the method further comprising:
receiving and storing, by the smart headset, diagnostic images of a portion of a body, wherein generating the at least one graphical element is further based on the diagnostic images of the portion of the body.

9. A method for orienting a surgical tool at a desired three-dimensional insertion angle at a desired location within an environment for use in installing a medical device by using and displaying at least one graphical element, the method comprising:
determining, by one or more processors, the desired three-dimensional insertion angle of the surgical tool based on an orientation of the surgical tool;
collecting, by the one or more processors, environmental data of the surgical tool within the environment;
generating, by the one or more processors, at least one graphical element comprising visual indicia for orienting the surgical tool at the desired location based on the desired three-dimensional insertion angle; and
displaying, by the one or more processors, the at least one graphical element superimposed within the environment on a smart headset communicatively coupled to the one or more processors.

10. The method of claim 9, wherein the visual indicia comprises a virtual tool for orienting the surgical tool at the desired location and the desired three-dimensional insertion angle, wherein the visual indicia further comprise a three-dimensional vector comprising a guideline indicating a trajectory of the virtual tool, and wherein the method further comprising:
generating, by the one or more processors, interactive elements for interacting with the smart headset;
displaying, by the one or more processors, the interactive elements superimposed within the environment; and
wherein the one or more processors are enclosed within the smart headset.

11. The method of claim 10, further comprising:
receiving, by the one or more processors, an instruction from an individual operating the smart headset; and
locking, by the one or more processors, the virtual tool superimposed within the environment, wherein the virtual tool is stationary at the desired location and the desired three-dimensional insertion angle as the smart headset changes positions within the environment; and
wherein the instruction from the individual is at least one of an eye movement, a gesture, an auditory pattern, a movement pattern, haptic feedback, a biometric input, intangible feedback, or a preconfigured interaction.

12. The method of claim 9, wherein:
the visual indicia comprises concentric circles indicating thresholds of the desired three-dimensional insertion angle of the surgical tool;
the concentric circles comprise a first set of concentric circles at the desired location based on the desired three-dimensional insertion angle and a second set of concentric circles indicating a live orientation of the surgical tool, wherein the one or more processors is calibrated to the surgical tool to indicate the live orientation of the surgical tool; and
the environmental data comprises orientation data of the surgical tool, and wherein the one or more processors continually collects the environmental data in real-time.

13. The method of claim 12, further comprising:
in response to continually collecting the environmental data, automatically updating, by the one or more processors in real-time, the at least one graphical element superimposed within the environment, and wherein the one or more processors comprises a gyroscope, and wherein generating and displaying the at least one graphical element is based on continually collecting, by the gyroscope in real-time, orientation data of the smart headset; and
in response to continually collecting the orientation data of the smart headset, automatically updating, by the one or more processors in real-time, the at least one graphical element superimposed within the environment.

14. The method of claim 9, further comprising:
capturing, by the one or more processors from an input device of the smart headset, additional environmental data of the environment, wherein the input device is at least one of a camera, sensor, or internet of things (IoT) device, and wherein the additional environmental data comprises orientation data of a portion of a body, and wherein the orientation data of the portion of the body indicates at least one of an axial plane, coronal plane, or a sagittal plane associated with anatomy of the portion of the body; and
determining, by the one or more processors, an orientation of the portion of the body within the environment based on inputting the orientation data into a machine learning algorithm and receiving an output prediction indicating the orientation of the portion of the body within the environment.

15. The method of claim 14, wherein generating the at least one graphical element comprising the visual indicia for orienting the surgical tool at the desired location is further based on the orientation of the portion of the body within the environment, and wherein the method further comprising:
generating, by the one or more processors, visual indicator elements indicating the orientation of the portion of the body within the environment;
displaying, by the one or more processors on the smart headset, the visual indicator elements superimposed within the environment; and
receiving and storing, by the one or more processors, diagnostic images of the portion of the body, wherein generating the at least one graphical element is further based on the diagnostic images of the portion of the body.

16. A smart headset for orienting a tool at a desired location within an environment, the smart headset comprises:
a transparent or opaque display;
a plurality of sensor devices; and
one or more processors configured to:
initiate the smart headset to be calibrated to the environment so that the smart headset knows its position relative to the environment when the smart headset moves in the environment;
collect, via the plurality of sensor devices, environmental data of a surgical tool within the environment using physical elements, fiducial elements, or geometric shapes of the surgical tool that is located at the desired location;
calculate an orientation of the surgical tool based on collecting the physical elements, fiducial markers, or geometric shapes of the surgical tool;
receive a desired three-dimensional insertion angle;
determine a position of the desired three-dimensional insertion angle at the desired location;
generate at least one graphical element comprising visual indicia for orienting the surgical tool at the desired three-dimensional insertion angle at the desired location; and
display, via the transparent or opaque display, the at least one graphical element superimposed within the environment.

17. The smart headset of claim 16, wherein the visual indicia comprises a virtual tool for orienting the surgical tool at the desired location and the desired three-dimensional insertion angle, wherein the visual indicia further comprise a three-dimensional vector comprising a guideline indicating a trajectory of the virtual tool, and wherein the one or more processors are further configured to:
generate interactive elements for interacting with the smart headset; and
display the interactive elements superimposed within the environment.

18. The smart headset of claim 17, and wherein the one or more processors are further configured to:
receive an instruction from an individual operating the smart headset; and
lock the virtual tool superimposed within the environment, wherein the virtual tool is stationary at the desired location and the desired three-dimensional insertion angle as the smart headset changes positions within the environment; and
wherein the instruction from the individual is at least one of an eye movement, a gesture, an auditory pattern, a movement pattern, haptic feedback, a biometric input, intangible feedback, or a preconfigured interaction.

19. The smart headset of claim 16, wherein:
the visual indicia comprises concentric circles indicating thresholds of the desired three-dimensional insertion angle of the surgical tool;
the concentric circles comprise a first set of concentric circles at the desired location based on the desired three-dimensional insertion angle and a second set of concentric circles indicating a live orientation of the surgical tool, wherein the one or more processors is calibrated to the surgical tool to indicate the live orientation of the surgical tool; and
the environmental data comprises orientation data of the surgical tool, and wherein the one or more processors continually collects the environmental data in real-time.

20. The smart headset of claim 19, and wherein the one or more processors are further configured to:
in response to continually collecting the environmental data, automatically update, in real-time, the at least one graphical element superimposed within the environment, and wherein the one or more processors comprises a gyroscope, and wherein generating and displaying the at least one graphical element is based on continually collecting, by the gyroscope in real-time, orientation data of the smart headset; and
in response to continually collecting the orientation data of the smart headset, automatically update, in real-time, the at least one graphical element superimposed within the environment.
